SEQUENCE LISTING (160 sequences)

SEQ ID NO: 1 - DNA, 322 bp, Artificial Sequence
  Bisulfite converted DNA (ID 2025 of Example)
aatcctccaaattctaaaaacataaaaataacgcaacccaaaaacaaaaaacccctccgcccattaattactatacactaacgaaactttcccgacccacaacgacgaaaataaaaacaatcgctaacgctaaaaaacatcaaaaacactacccaacccaaatatcgccgccgcttccacaaaactctactaaacgccgccgccgccgctaccaccgcctctaatccaaaccacctcccgccaaataaaccccgaaatcctaactcaaatatatatctctccctccctctccctccattcgtcattttctcactccctttcc

SEQ ID NO: 2 - DNA, 413 bp, Artificial Sequence
  Bisulfite converted DNA (ID 2044 of Example)
ggataggagttgggattaagattttcggttagtttcgtattttttcgtattttttagtatcgtttcgtatttttcgtattttttttcgggttattacgttttttatgtgattcgtttgggtaacgtcgaatttagtcgcgtagcgttgtagtgaattttttttttaaattgtaataagtcgttttttaaggtaattacgttttttttgttttttttttaaaaaataaaaataaaaaatttatagaaaaaaattcgcgagtttagaaaaaagaagtaattggtagaaggttttaattaaggtaaagagttgtaaggcgaagttaagaaaatgtaggtatttaaaaaatgtaggtaatttttataagggtttttggggagaggtatatagagggattttggtgttgaaaaagatt

SEQ ID NO: 3 - DNA, 347 bp, Artificial Sequence
  Bisulfite converted DNA (ID 2045 of Example)
aaccctttcttcaaattacaaaccttcttaccttcaaacctcgactccaacaccaatccgacaaaaaaacccaatctaataaaatacgctcccttcctaccattctctattccattaacctatttcgtaataaacgtaaaactaatcctccaaaattaccttattaattaacttacatatttattatctatctatcccaccaaaatacaaatttccgaaaaacaaaaatttaaaaaaatctattttattctatataattttcccataccaaacaccgtacccgacacaaactaaaatcccaatacacatctcgaaacgaaaaaaccgtatttccctaaaacccaatc

SEQ ID NO: 4 - DNA, 283 bp, Artificial Sequence
  Bisulfite converted DNA (ID 2106 of Example)
ttgaaaataagaaaggttgaggtagagaggataatatagttttagtttattttttagtattttgttaattttttttaatttttagttataaattcgagatataacgttttttttttaaagaggtcgcgttttttttgtggtggtttttagggattcgttttagtttttttttcgtttttagttttatatattgggattattaggtatttaagattttattttttaggtggtatttttagcgtaggttgttatttagtttttttttagggatttggggtagaag

SEQ ID NO: 5 - DNA, 211 bp, Artificial Sequence
  Bisulfite converted DNA (ID 2166 of Example)
tgtttgggattgggtagggttatcggggttgggggggcggggtttgtgggtaaggcgggcggaggcgtggatttttcgttcgatgatagggttggaggaggaaggggcgggttgaagaaggggaaggtgggaagagtttagtcggggttataaattgggtgaagcgttgaggttttagtattttcgtttgaggagataggtaaaggttatg

SEQ ID NO: 6 - DNA, 497 bp, Artificial Sequence
  Bisulfite converted DNA (ID 2188 of Example)
ttttagattgaggttttagggttaaaggattatttttttttttagcgttggttcgggaaaggtaagtttcgggcgggagcgtacgtcgcgttttcgaagtttggttttttcgttacgtttattttttgtttttatttcgcgtttttttaggttttttttcggtgaatcggatgttttgttagttttttattttgcgttttcggtcgcggttcgggtttttcgtaaagtcgttgttatttcggagggtttagttagcgggttttcggaggttggtcgggtaggcgtggtgcgcggtaggagttgggcgcgtacggttatcgcgcgtggaggagatattgttttgtcgcgatgggggttcggggcgtttttttacgtcgtaggtaagcggggcggcggttgcggtatttgtttatcgggagttttttttttttttttttgttgttgttgttttgtatttagttcgggggaggatagaagaaaaaggaggtagaatggat

SEQ ID NO: 7 - DNA, 373 bp, Artificial Sequence
  Bisulfite converted DNA (ID 2191 of Example)
ggaggggagagggttatgcgattttatttttggttagggtcggggaggtttttgtttttcgggagttttgttcgggttttttggtcgtagggttgttgggttttaggtaggaacgagagggtgaggtttatatgtggttcggcggtttagggcggtttgtagcgtttttattgtttcggttgttaggggttgcggcgacgcggttagttagtagcgagtttaggtcgcgtagattttattgatgagttttgatttttagtattttttttaagttaagaagagtttagcgtatttttcggttgttttattttagtttttttgttttagttttttagttttattttttttcgttttgttttggggtgtgtatagt

SEQ ID NO: 8 - DNA, 368 bp, Artificial Sequence
  Bisulfite converted DNA (ID 2194 of Example)
ttttgggaatgggttgtatcgagaggttcgattagttttagggttttagtgagggggtagtggaatttagcgagggattgagagttttatagtatgtacgagtttgatgttagagaaaaagtcgggagataaaggagtcgcgtgttattaaattgtcgtcgtagtcgtagttatttaagtgtcggatttgtgagtattttgcgtttttagttttcggatagaagttggagaatttttttggagaatttttcgagttaggagacgagattttttaataattattatttttttttgcgttttttatttgtcgttcgttgggataaacgatagttatagtttttttgacgataggatggaggttaagggta

SEQ ID NO: 9 - DNA, 352 bp, Artificial Sequence
  Bisulfite converted DNA (ID 2212 of Example)
ttgttgggagtttttaagttttgtgagaattttgggagttggtgatgttagattagttgggttatttgaaggttagtagttcgggtagggtttatcgaaagtttattcgtatatattaggtaatttaattttttattttgtgtgatagaagtagtaggaagtgagttgtttagaggtaggagggtttattttttgttaaaggggggattagaatttttttatgcgagttgtttgaggattgggatgtcgagaacgcgagcgattcgagtagggtttgtttgggtatcgtcggggtaggattcggaacgtattcggaaggttttttgtaagtatttatttggaaggagaattt

SEQ ID NO: 10 - DNA, 295 bp, Artificial Sequence
  Bisulfite converted DNA (ID 2267 of Example)
gtaatttgaagaaagttgaggggaggcggtagatgttttgatttattagggaaaacgtggacgttttttgttgttattttgtgaattgtgtgtatttagttatttttgagtaaatatttggagcgaggaatttttgagtggtgtgggagggcggtgaggggtagttgaaagtcggttaaagttttcggaggggttggtttaggaaatatgattggtagttacgagagagttaggggttggacgtcgaggagagggagaaggttttcgggcggagagaggttttgtttagttgttg

SEQ ID NO: 11 - DNA, 278 bp, Artificial Sequence
  Bisulfite converted DNA (ID 2317 of Example)
ggagttgtattgttgggagatttgggtgtagatgatggggatgttaggattattcgaatttaaagttgaacgtttaggtagaggagtggagttttggggaattttgagtcggtttaaagcgtatttttttgtatatttattcggtgttgggcgtagggaatttttgaaataaaagatgtataaagtattgaggtttgagatttttggatttcgaaatattgagaatttatagttgtatattttagagtttatggtattttagtgaaaattggggtttt

SEQ ID NO: 12 - DNA, 285 bp, Artificial Sequence
  Bisulfite converted DNA (ID 2383 of Example)
tttgtattaggttggaagtggtcgttagtttttcgtgtaattttattttttggaaaagtggaattagttggtattgtttagcgtgatttgtgaggttgagttttaatagtttaaagaagtaaatgggatgttattttcgcggggttcgtttttcgcgaggtgtttatttcgtatttgttatgtaaaacgagggagcgttaggaaggaattcgttttgtaaagttattggttttggttattagtttttatttaatgttttcgtgatgttgttgttgatttatttgg

SEQ ID NO: 13 - DNA, 380 bp, Artificial Sequence
  Bisulfite converted DNA (ID 2387 of Example)
gatttttggagaggaagttaagtgtttttttgttttttttcggtattttatttaaggcgattagtttagaattggttttcggaagcgttcgggtaaagattgcgaagaagaaaagatatttggcggaaatttgtgcgtttggggcggtggaattcggggaggagagggagggattagataggagagtggggattattttttttgtttttaaattggggtagttttttgggttttcgatttttttattttcgtgggtaaaaaattttgtttttatcgggtttacgtaatttttttaaggggagaggagggaaaaatttgtggggggtacgaaaaggcggaaagaaatagttatttcgttatatgggtttggtttttagttt

SEQ ID NO: 14 - DNA, 397 bp, Artificial Sequence
  Bisulfite converted DNA (ID 2391 of Example)
tggggttagtttaggataggcgttcgggggacgcgtgtttttattttacggggacggtggaggagagttagcgagggttcgaggggtaggtattttaacgaatggtttttttggtgttttttgcgtttcgtcggtttatttttttttttataaaacgggtttagtttttagtatttatttttcgttattaattaggtatttcgggagattagttcgttcgaaagtttttgcgttatttcgcgggtttttttaggtggtttttttagtttcgttttttttcgggatgtttgttgattatttcgagttcgcgtggcgtaagagtacgagcgtcgagttcgtgcgcgttaaggttgcgtgggcgggtatcgatttttttgagaagttttagtgtttttaa

SEQ ID NO: 15 - DNA, 547 bp, Artificial Sequence
  Bisulfite converted DNA (ID 2395 of Example)
tttttgtattggggtaggtttcggtaggtgtatgggaggaagtacggagaatttataagtttttcgattttttagtttagacgttgttgggtttttttcgttggagatcgcgttttttttaaatttttgtgagcgttgcggaagtacgcggggttcgggtcgttgagcgttgtaagataggggagggagtcgggcgggagagggaggggcggcgtcggggcgggttttgatatagagtaggcgtcgcgggtcgtagtatagtcggagatcgtagttcggagttcgggttagggtttatttgttttcgtagcgtcggttcgcgtttttttgtcgtagttatcggtgagtgtcgcggttttgagattttcgggtcggatgcgcggcggttttagttttcgagcgtttgtttgtttcgttttgggttgttcgggttttttgggtttttcggcggttgtacggagttaaggcgtttcgtttcgggcgtttttcgcgggtgtcgatttaggttgttcggagttcggagtttatagaggagagagatagttgg

SEQ ID NO: 16 - DNA, 414 bp, Artificial Sequence
  Bisulfite converted DNA (ID 2401 of Example)
attagaagtgaaagtaatggaatttcgatgtaaatataatattatttttttgtagagttattttgagtataataaatttgaattgtgttaatgttgggagaaaaaatttaaaagaagaacggagcgaatagtagttttttcgttcgttgattagaaatagtaggacgatattttttcgattggaggagagcgtttgcgttcgtatttagttggcgttcgtttttttgttttttttttagtcgtttttttttttttttttcgcgttttagttattcgggaaggtattgcggtagttgggttttgattggttgttttgaaagtttacgggttattcgattggtgaattcggggttttttagcgcggtgagtttgaaattgttcgtatttggttttaaagttggtttttggaaattg

SEQ ID NO: 17 - DNA, 272 bp, Artificial Sequence
  Bisulfite converted DNA (ID 2453 of Example)
gggatgggttattagttgtaaatcgtggaattttttttgatataatgaaaagatgagggtgtataagttttttagtagggtgatgatataaaaagttatcggagtattttataaggtataaatttttagagatagtagagtatataagtttttaggataagagttaggaagaaattatcggaaggaattattttattgtgtgtaaatatgatttttaagttggtcgtggtttttttggtagtttttttgatttttgtagttttgtgtgaagg

SEQ ID NO: 18 - DNA, 391 bp, Artificial Sequence
  Bisulfite converted DNA (ID 2484 of Example)
taattgaaggggttaatagtggaatttggttgggtgtttgttaaatttttttttttggttttgttttgggtttttttttgaagggattttttttcgtttttgtaataagattttttataaagtatagattttttattttatttcgcggtatttgtatcgggttttattggttttaggagttgaatatttttttaggtatatataggtgggatataaataagggttttggaattattatttttttattacgatagtaatttaaaatgtttgggaagatggtcgtgatttttggagttttaaatatattttggataatgtttgtagtttgtaagttatttttttttatttgttttaaatgttagtatttaattttagttttggttttggtttt

SEQ ID NO: 19 - DNA, 430 bp, Artificial Sequence
  Bisulfite converted DNA (ID 2512 of Example)
agtggatttggagtttagatgtaatataatgattgatattggtatagtatatttattttgtttttgtaaataaaatggtatatgtgatgtttttttttgtttttttgtatataaaataatatttgtttttatttattatgtatttatgtttttattttgtatgttaggagttaagtattttgtatgtattaatttattttgtttttataataatttttatatgtaggaattattatagttattttatgaatgagtcgaggaaggtattgagacgttaagtaatttgtttaaggttacgtagttagtaagtggtagagtaagaattattatggttttataagtttaggaaaaagtttgaaagaattaaaatgttaatagcggggattttaaggaagtattgaagaggttatgggagaagtttttattttgt

SEQ ID NO: 20 - DNA, 475 bp, Artificial Sequence
  Bisulfite converted DNA (ID 2741 of Example)
taggggaaaagttagagttgagaggttggggcgcgacgagtttggatatcgggcggggatttaagtttttttcgtttagttaataattgtgttttttttaggaaggcgtgaggaaatgttttaattaatttttgtattttttttttggaatttgggttgtatttttttatttattgtaaattttataatttatttaggggtttttttagtgtttgtttttagcggtttcggtgtttatttattagtgttgttttttttttttcgtaagattgcgttttagttttagttttttttttcgcgggtgttttttaaatcgttttattattttcgggtttagggaggcggaatcgtgtttgtttttcggtttttttaagaggcgtcggttttattttttttagagtcgcggtttgacgcgagatgatagtaacgagttcggtatgtttatgtaaataagcgtttttttgtgggttaatgg

SEQ ID NO: 21 - DNA, 412 bp, Artificial Sequence
  Bisulfite converted DNA (ID 2745 of Example)
attttagtttgtgaaatgggatttaggatttaggtagaggtgcgttttcggtttggggatcgagtattttgtgcgtttcggtaacgtaggaagatagcgttattgatattttagagattagcgggtatcgtttggaggcgtttttattatttggcggtttcgggttcgcgttttatcgcgttataagatttacgttcgaattacgtgattagggtcgtggtttcgtttcgttttcgcgtcgcgcgtcgttttcggtaggggcggaaagcggaagtgtgggagggtttgcggggcgggtttaggaggttcgcgggaggatggagtagtgagcgggtttgggcggttgttggtagcgttatggagacggtatagttgaggaattcgtcgcgtcggtgaggggttattggttaag

SEQ ID NO: 22 - DNA, 484 bp, Artificial Sequence
  Bisulfite converted DNA (ID 2746 of Example)
gtgggttttgggtagttatagaagttatcgcgttggcggggaggagggggatcgatgcggtttatgtttcgggtagttttattttttttgtttgcgaagggtttttgttcggcgggaggagagaggcgcgttttattcgggtttttttatatttgtcgtcgtttgggtcgatttcgcgggtttcgttcggcgttttagtcgattttcgtttagtttcgggtttatgggcgcggttagtagggcgggttagggcggcggggcgcgatattgggaggaagtgcgggtcgtttgttcgggcgcgttaaggaagttgtttaaaatgaggaagagtcgcgggttcggcggttgaggttatttcggcggcggttggagagcgaggaggagcgggtggtttcgcgttgcgttcgttttcgttttatttggcgtaggtaggtgtggtcgcgttttttattcggtcgggattttttggtaaggagaggaggtt

SEQ ID NO: 23 - DNA, 476 bp, Artificial Sequence
  Bisulfite converted DNA (ID 2747 of Example)
taggatggggagagtaatgttttcgagtagaatagggtggggtttttagattatttttttttttttatagttggttttattttatcgattttattaaagtttttttgggagtattttagagaagagttacgtttaggtcgggttttggttgtttggtttacggcggaatttttagtattacgtttcgtacgtcgggtttaaagtatgtttagtgaaggagtaggtatttattgttagatggagttatttttttagatttggggtttttttataacgatggttatgtttggtatggaagtttttttagaagttaatagtaggaaataagggttaatagtatttaattgtggagtaaggtttaaattttagttttgttatttaatcgtttcgaatttgtttttttattgtagaggcgaaaaggttaatattattttatttcggagggttatcgtggagaatggaagttggataagttg

SEQ ID NO: 24 - DNA, 419 bp, Artificial Sequence
  Bisulfite converted DNA (ID 2749 of Example)
tcccacaaaaactaaacaattattacaaattcaaaaaaccccgaccaatttttcaaaaatttctcctcctcttttccccctaaaactcgtaatacttttactctactttcaaaatacattaaatctcctactttataactactttaaaaccaacaaatactctaatatatataattcaaattatacaaatttcacgaataaatttaatcttattttttaaattaattaaaaaacaaataatatttaaaaaaatattaacttataattatttcaccctttttactttaaacatttttattacttctcgaccttttaactaaaatcaaatatatactttaaacattttttaaaataaaaatatccttttaatttaataaaaaaacaaaattctacataaaaaaaccccttcatctaaaacc

SEQ ID NO: 25 - DNA, 479 bp, Artificial Sequence
  Bisulfite converted DNA (ID 2751 of Example)
tttggagggtttagtagaagttattttaggggagggttcgataggaaggaaggtaggtttgtcggaggggtatataggagttttttttttcgttatagtgtttagggttaattgttttagtttttaggttgggttaataggatgggatagtttaggcggaaggaaatttgtggggagggatatttcgtagatagaagtagggatatggggtggggagaggtaggaagagttgtcgggttgttgagttggcgtttttttagtagatttaggaggggcggtgataggaggttattttttttttattttcgtagttttgggtttttttggttttggttaatagtattattattattattattgttgttgttcgttagtttgggttttagatatattagaaaaaaattatcggaagatacgtatagtattggtagtttttaaaagaattaattttttttttgtgtttattttgtgattattgg

SEQ ID NO: 26 - DNA, 484 bp, Artificial Sequence
  Bisulfite converted DNA (ID 2752 of Example)
atacaacctcaaatcctatccaaacccccaaaacatcacactcgaaacttattctacatatttttacttttacctcccactaatactaattcttccgtaaaacaacctaaatcccttcaaatacttaatattttttctcaaatactaccataaaaccaaatctccaccgtcttaaaacattcctttttaaaaataaaaaatatatatcgctccttttatataatttacattctatcttaaataatttaaccatcaccgtaattcattcaaatctatttaaatcctacccatctcaacttcaatccatttcattcttttaaatctaatcgacaattacctccaacaacttcatcacaaatcactcacaaaaataaccttaatcctaaaatttatttacgaaaaacacacttactaaatatataacaaatatacaaaaaacacaaaataaaacaacaaatctaaaaacaaataacttccttctccc

SEQ ID NO: 27 - DNA, 371 bp, Artificial Sequence
  Bisulfite converted DNA (ID 2755 of Example)
ggaagatgaggaagttgattagatattaaggatgagcggatgatttaataggtttttttgttaagatttggttgggtaggtgaaagataaagtcgaggagtggttatggtgtggtatagaagaagggttagaggacggtttttgttatttttttatgtttgagtttttttttttgtgaaatggggataataagagtcgttatatagggaattgttgttaggattaaatgagataatgtatgtgaaacgttttggttgtaggttttttagtaaatgggtacgatttgcggagtggggatttgaatttacgtttggcgggatgtttaagttgttattttgatcgttagggagttttagaggatagggttgtag

SEQ ID NO: 28 - DNA, 186 bp, Artificial Sequence
  Bisulfite converted DNA (ID 2831 of Example)
ttagtaggggtgtgagtgttttgattagaattatttttttttgttagaatttgatgtaattcgaatgtttttatttttgtttgaagggtttaaataataaattaggttttgtcgtgttattatgggggtggttatattttgtatttaggaaataggtacggtagggttgagatagaagttttgttt

SEQ ID NO: 29 - DNA, 300 bp, Artificial Sequence
  Bisulfite converted DNA (ID 2850 of Example)
ttatagggttgagtttgggatcgaggtgagagtcgtcgggttgggagtgagggagatgggaataaggtcgtcggtgggcgaggggagtcgagggaattcgggggattgggaggtttggggcggcgcggtttggtcgggttgggatcggtttttcggtttagacgttcgcgatgttggtattttttgttattttttatttgggttttaggggttcgtttttgggtagtttggagtttttcgaggtgggaggatcgggcggaggtggaggaagtttttttttggaagatttgttgtttgttt

SEQ ID NO: 30 - DNA, 321 bp, Artificial Sequence
  Bisulfite converted DNA (ID 2852 of Example)
tgaaaatgaaggtatggagtttggtgttaaaagaaattttttttaaaaattaaataataatattagagtaaagtttttagggcgagataaggagttgtaataaaataagcggaaattcgagaagcgttaatgttttaaagggttaatgattatatataatttacgtagttaacgtgttaaaatatattaacgtattttttttttttaaataaagtaggaaagcggattttgtatgaggggcgggttgtcgatttagtagtttttttcggatagttcgttttgattttttttggttggtcgtggagggattatatggtttta

SEQ ID NO: 31 - DNA, 398 bp, Artificial Sequence
  Bisulfite converted DNA (ID 2859 of Example)
tatgtttggttttgttttgagatagagtttcgttttgtcgtttaggttggttaaaagatagggttttagtcgggtgcggtggtttacgtttgtaattttagtattttgggaggtcgaggcgggcggattatttgaggttcggagttcgagattagtttgggttaatatggcgaaacgttgtttttattaaaaataataaaaattatttaggcgtggtggcgcgtatttgtaattttagttattcgggaggttgaggtaggagaattatttgaatttaggaggtagacgttgtagtgagtcgagatcgcgttattgtattttagtttgggcgatagagggagatttcgttttaaaaaaaggaaaaaaaaaaaaaagaaaagaaataaaagtgatggggt

SEQ ID NO: 32 - DNA, 347 bp, Artificial Sequence
  Bisulfite converted DNA (ID 2861 of Example)
gggtgtagaagtgtttaggttttttttcgttggggttgggagtttgggtaggttagttttattttttttaagttcgtttttggttttcgggtttagtttcggttattatgtttcgttagattatttttgtgggttttagttgtttggatttgtggagggaaaagaatgatcggttcgttcgataggttaaggtaatacggttgttggtattttcggtttgtagttttaagatttttgaaagcgggtttgtagtggatttattttaatagatggggagggattgagtttgattaaagagttagaaatgattggagaatgtattttttgttattgttgtaaggggagaa

SEQ ID NO: 33 - DNA, 291 bp, Artificial Sequence
  Bisulfite converted DNA (ID 2864 of Example)
tccccttccaactatatctctcacccaaaaataacttctaactctcgtattcatctaaaactcctccttccatataccaacaattaactataacccctccaaaaacgctccatctccaaatatactcccacatccaaaccacgaacccctcacccgatcacatacttcatacacctataactccgcactccccaaatatacctctaacgtacaactattaccccttcccccgattataaccctataactcgccacatacaactataactaaaacttccctaaaacactctc

SEQ ID NO: 34 - DNA, 389 bp, Artificial Sequence
  Bisulfite converted DNA (ID 2867 of Example)
aaaaccaaaacataaaccaaaaaccaaactcgaaccgaaaacaataaccgcaacgcccgaaaactaaacccacgacgcgctaacaacgcgaaccgaactacgaaaacgatcacgtcaacgtccgttccaaaccgactaacaatctccgttctacattaacgtcaacactcccgttaaaaataatacatctctcccataccaaaaaaacttaaatactactaaaaaccaaccctccgaatactaccaaaccgacgctcacccgccaccttcatcttcccttctcctttaccccaaaacaaccgaaaatatataattaaattccccctacccataaaaaaaccaaaaataaaaaactaacgacctactcgatctcaacaaaccctcctaat

SEQ ID NO: 35 - DNA, 272 bp, Artificial Sequence
  Bisulfite converted DNA (ID 2961 of Example)
aatggttgatgattttggttttttttcgtcgtcggagagcggtgtttcggaggcggcggaggaggattcggcggtcgtttttttggtttagtaggagagcgagattgtaggtatagagaacgacgagggtttcggggtatttgtcggtagttatgcggttttcgcgtagtcgggttttacgagtgggggtgagttagcgcggggtttggagaggggtttagggcgcgtattcgggggatttcggtcggggtttaggggtatagggaagagag

SEQ ID NO: 36 - DNA, 371 bp, Artificial Sequence
  Bisulfite converted DNA (ID 3511 of Example)
agttagaagaggagttaggatgggtttcgggtagtttaatagtatagttgaagttttaattattatgttaatagttttttggttttatatattttatgggaagaggaaaataaaaaggtatttatttgtatatttttttatttttgatataagaagtagaattttttttatatgatttatgtttatttaatacgttattttgaaatttattaataaaattttttaagcgttagaaaattgttagtggttttttttatttttttttatttttttttgtgttattaattttgttttttttttttagaaggttgtcggaatagtaaatatttattgatatgttataattattggaaaatgggtattggaaaatt

SEQ ID NO: 37 - DNA, 457 bp, Artificial Sequence
  Bisulfite converted DNA (ID 3532 of Example)
tgttagtagagttttagggaggttttattttttatttttatttaaagttttatttgttggggtgggggttttgtttggaaggggaaggtttaaggttgtttttagcgtgtttttttattttgattgtttttggcggggcgggggtgtttttgttatttagttgtataacggttaggaagggtttaaattatttttagggttaatttaaggtcgttttttgggtttgtatatttttgtgttgagtgcggatcgggagaggttgttgaagataggaggggataaatgggggacgaaggggttcgagggaggggattgaaggatttgggttaagtcgggagttttcgagggcggagttaaaacgtatttggattttgttagttttaaattttgtttttattgttgtaagttttttagatcgaggattttcgggttgagggtggggtaaggataggtagtg

SEQ ID NO: 38 - DNA, 476 bp, Artificial Sequence
  Bisulfite converted DNA (ID 3534 of Example)
tttttgtttttatggggtgtatatttaagtagttgaaatagatagtgaataaataaaaaaggataataattttaaataataatgatgttatcggttaggtgtggtggtttatgtttataattttagtattttgggaagttaagttaagcggattatttgaggttaggagtttaagaatagtttggttagtatggtgaaattttatttttattaaaaatataaaaattagttagatatggtggtatatatttgtaattttagttatttgggaggttgacgtaggagaattgtttgagttcgggaggtggaggttgtagtgagttaagatttgataggtttttagtattattgtattttagattggttgatagagcgagattttgttaaaaaaaaaaaagttataaatagattttaatagggtaatatgatagggagggagggataggggagtagggtggttaaggaagggatattta

SEQ ID NO: 39 - DNA, 458 bp, Artificial Sequence
  Bisulfite converted DNA (ID 3538 of Example)
tgggtagtatttttgttggtttttttttatattataaggttacgtagagttggcggagggttatggttttatttatgttaggtgtttttaatttggtaaggaaatgtaatttacgtgaattttaataggtagtgaagtatcgtttttttttgattttaggtagggtgaagaaaatgggatagtagtacggggtgcgggtataaacgtataattttgtttttttagacgtagagttgtggggttgtgagaatgttaggaggaggtaagaaagggcggttttatggggggtttgtagggtgggataagtttaagaggtttttatatttaggtttggtgggggaggtgagtttttggtttatcgagggggtttttttttgttttcggaaatattgtagtttttatttttatcgttttttcgttgcggggatttaggggcgtgaggatgagagagtttttag

SEQ ID NO: 40 - DNA, 405 bp, Artificial Sequence
  Bisulfite converted DNA (ID 3540 of Example)
agtggtttaggagtatttggttattttcgggaaaaatcggtttggtaaaggttttttcgagggtacgcgtttttcggatagtgaggtaggatttaaattttttcgttaatattatatttttcgtatttttgtagtgtttgtatttttaggttttattattttttcgtattttttagggagaagttttcgacgttttattttttttggaagggtgttgtttttagagatttttaggttaatggtttaattttagtgtttttaggggagaggggggtgtagaaaaatagtttgggttataaaagaggtgcgagggttgtgagatttcggaggtatcgacgggaagcgagacggagaataggagggtaggacgggttggaggtgggggatattgtagatggagggagt

SEQ ID NO: 41 - DNA, 2501 bp, Homo Sapiens
ccagttccagtcccgggtcctgtggccgccctgccggcgaccctgcggagagcgagtcttagatacccagtccccagccccgagttgttattccctcgctgtagttaagaaggaggagatcaattaagggcatcttagaagttaggcgttcccgctgcctcctttgagcacggaggccaccaaccccctagggggaagagatgtagcgcgaggcaggggtgtcgtgctaagaaatttcgacgcttctggggactgaggacaaaggtgcggacacgaccccggggtacctggagttccgtgactcgcgccacggacggcacacctaggggctaatttctgctctgcctcaaagaacctcaagctagagtccttgcctccgcccacagccccgggatgccgctgctgcgctcaccgcacaggcagcgcccggaccggctgcagcagatcgcgcgctgcgcgttccaccgggagatggtggagacgctgaaaagcttctttcttgccactctggacgctgtgggcggcaagcgccttagtccctacctctgctgagctgaacgctcaggcacagtggaactgaaacccggttctgcgggatgtgagagctgttgaggtcacgcgtaattgggtgtgatggagggcgcctgttcgtgatgtgtgcaggtttgatgcaagcaggtcatcgtcgtgcgagtgtgtggatgcgaccgcccgagagactcggaggcaggcttgggacacgtttgagtgaacacctcaggatactcttctggccagtatctgttttttagtgtctgtgattcagagtgggcacatgttgggagacagtaatgggtttgggtgtgtgtaaatgagtgtgaccggaagcgagtgtgagcttgatctaggcagggaccacacagcactgtcacacctgcctgctctttagtagaggactgaagtgcgggggtgggggtacggggccggaatagaatgtctctgggacatcttggcaaacagcagccggaagcaaaggggcagctgtgcaaacggctcaggcaggtgatggatggcagggtaggaagggggaggtccagaggtctggatggaggcttccgcatctgtaccttgcaactcacccctcaggcccagcaggtcatcggccccctcctcacacatgtaatggatctgaagagtaccccgggacagtccggggagatggagattcggaaagtatccatggagatcttacagaatcccctgtgcggaccaggaaactcttgtagatccctgcctatctgaggcccaggcgctgggctgtttctcacaatattccttcaagatgagattgtggtccccatttcaaagatgagtacactgagcctctgtgaagttacttgcccatgatcacacaaccaggaattgggccaactgtaattgaactcctgtctaacaaagttcttgctcccagctccgtctcttgtttcccacgagccctggccctctgtgggtaataccagctactggagtcagatttcttgggcccagaacccacccttaggggcattaacctttaaaatctcacttgggcaggggtctgggatcagagttggaagagtccctacaatcctggaccctttccgccaaatcgtgaaaccaggggtggagtggggcgagggttcaaaaccaggccggactgagaggtgaaattcaccatgacgtcaaactgccctcaaattcccgctcactttaagggcgttacttgttggtgcccccaccatcccccaccatttccatcaatgacctcaatgcaaatacaagtgggacggtcctgctggatcctccaggttctggaagcatgagggtgacgcaacccaggggcaaaggacccctccgcccattggttgctgtgcactggcggaactttcccgacccacagcggcgggaataagagcagtcgctggcgctgggaggcatcagagacactgcccagcccaagtgtcgccgccgcttccacagggctctgctggacgccgccgccgccgctgccaccgcctctgatccaagccacctcccgccaggtgagccccgagatcctggctcaggtatatgtctctccctccctctccctccattcgtcattttctcactccctttcctcctctccctctctctccgttagtctcttcatcagatagtctctgttagtccgcgatttataccaggctcgtgccctaggttggatcggacagtctcaatcccccggctcgctcttcctgctcggctgcggactccagtcttactctctcgcactgcacacaggcttaggccagtctcgggacactcaggctccccagggaccgcgcacagagcctgaggcaagagaaactttccgcagacggtgcgatcagggacggcgtctggagcccagcagtcccagggaaattggttcagaacctggaacagagcggatgggtggcaaataggcacgacgactgagggacaagcagccctaaactgca

SEQ ID NO: 42 - DNA, 2501 bp, Homo Sapiens
agatttactcaaatttaagaatgagaatacaaatccacatcttgaagtgtttcacagaaaggtctatcttaatgtctggagtatatatttcaatgaacattcattttattttatttctctccattcctgaatcaagcaatcttgaatctaaagttgctatgattagcactgaaaagaccactggactattaattgtgtgactttgggacagtaactttctgcaccttagtttgtttacatgttatacatgaaggttgaagtctgattctgctctgtgactatcattctaaacatctgatgaaatcaaatttcagtgtttggaatggtagtacaataaatttactaagaataaataattcactgcaaaaacacattgatttccaaatgatgtaactgacagttatattactgcagagggctgataaataacaaaagaaatgaaagatgcacatggtgagaactgaaattatcctgacaagtcttctacctgtttatcacttaaaatcaatgaccatgctgaatgcctacaaattacaaaatataaaagaaatcttataaatgcgcatgtacaggagtctaagttactaaaagttttaaagcataagtttaaaccaaactaatcaaagaagttgagaggaaaaattggctttcatctttaatcactactgttttgaggtcctatgtttaatataattttctaagtagaggcttcagagagaagagttgtgaggatactttcatatttgtgtagaaggaaaagtttgccatccattctagtatccctagtgttatactgatgtgcaccttggatttattttgttcctattgtataaactcatacttgacttcaaagaaaaggaaaatccaaagtccctcttttctaaggggacagaaatcctttgtgtcaactgtttgacccttttctctgtaaggtcctattggaaatcttttgtaacacaatgcaggggactcttccatgtgttgatgctgtttacacagtggggtgggcctgactgaagaaaaaaaatcgcatatacgcatgaaagattatggtcttatttccggaaagcatgaaaggtgattgatacttccaagaagtccctgttactcaggaaaattatcaaatattctactcagagatacttggaaagactgaaggaaaggaagaacgaagaaagcagaatctagacttatgtggggagagatttgtggcagaggaaaagtattctctttgaatccgacaagggatttgcctgggggaatttcctgtccagccttttattaccagggtcttttgaagccgggctccccattgggcagttccctgggagtgcagtggggaattcttacactttccctctaggtccccgaaggatctcgttttctcagtgtctctttcaggttggcaggagccttgagcctgacacttccctttgatgggacaggcaagctctgtgggcgcgtaaacacgctgtaaccaagttctttgctgattttacagttttgtgtgctcccgagaagaagtgatcgtactcaattgtctattgctggcctgccccctaagagcctgggggctcctttcccctaacccagaactagctgcacggggggcggggaaatgggggtggggaaggagtgggagggcagtggtttccgcgagcagagcgatgttactgagtgagtccctgaatggggagcgctgctgtccccaagccgattggtacttcttgtcaggaagaaacgccaagaggtgggagtgcctggggagggaggcaggcggtccctaccgcaggcgcggggagctgcctttccgcccctccgcctgctttccaagcctggactcttaggagtggctgaagctgcggagcgcttttggagcctgtgaatgaaccctcctcctctccctcctccttcttctcgctgagtctcctcctcggctctgacggtacagtgatataatgatgatgggtgtcacaacccgcatttgaacttgcaggcgagctgccccgagcctttctggggaagaactccaggcgtgcggacgcaacagccgagaacattaggtgttgtggacaggagctgggaccaagatcttcggccagccccgcatcctcccgcatcttccagcaccgtcccgcaccctccgcatccttccccgggccaccacgcttcctatgtgacccgcctgggcaacgccgaacccagtcgcgcagcgctgcagtgaattttccccccaaactgcaataagccgccttccaaggtaatcacgtttcttttgttccccccttaaaaaacaaaaacaaaaaacttatagaaaaaaacccgcgagcttagaaaaaagaagcaattggtagaaggctttaattaaggcaaagagctgtaaggcgaagttaagaaaatgtaggcacttaaaaaatgcaggtaactttcataagggcttttggggagaggcatacagagggaccttggtgttgaaaaagattcagacaaaagaaacccag

SEQ ID NO: 43 - DNA, 2501 bp, Homo Sapiens
tgtgggtcattaatgcaatgttatttaagactaggatttggctgggcgcagtggctcacgcctgtaatcccagcactgtgggaggccgagccgggaggatcacctgaggtcaggagttcaagaccagcctgaccaacatggtgaaaccacgtctctactaaaaatacaaaattagccgggcatagtcacatgcctgtaatcccagctactgggtagcctgaggcaggagaatcgcttgaacccgggaggcggaggcggagtttgcagtgagccaagatttcacaactgcactccagtctgggccacaagagcgaaaacccgtctcaaaaaaaaaaaaaagactaggatttgacataaggcctgaggggtattcttttgttttgttttgccttgttttcaagaggccaaaatcttcacagttgaaaatttctgttgaaccacagagatttgaaccaactcagtttagaaagcctggggatttgaacaacggtatggatcggaaatctcttcatctgtcagttttcatcattctaggcagtaaaatagatttccctttaggagcttttcaccgtttggggttctccagcagtgggatgtggggaatcaacccttcttcgtctccacccaaacattaggtgggagcaaggggtgggaagtagagaaagtggatagaggtctccagtggatatgggatctttgtgtagaccagcacagtcctcagaaatctcatgcaagcaacataggtactgttatattttctagtggccaccttttaaaaagtaaacaggtgaggccgggcgcggtcgtcacgcctgtaatcccagcactttgggaggcccaggcgggcggatcacgaggtcaagagatggagaccatcctggtcgacacggtgaaaccccgtctctactaaaaatacaaaaattagctgggcatggtgacgcgcgactgtagtcctagctactggggaggccgaggcaggagaatcacttgaaccctggaggtggaggttgccacgctccactacactccagcctggcgacagagtgagactccgtctcaaaaaaaagaaagtaaacaggtgaaattaattttaataatatattttgtttaacccaacgtatccaaaatactatcatttgaaagtgtaatgaatataaaaatattcatgagatatttttcattctcatatccatactgtcttggactctaatgtgtattttacacttacagcacaattaatttgggactagctacatttcagctcaacaatagccaatagcatatgggatagcgcaaataaactctgcgtctctgttgcttctttgggtctcggagacctcaaccctttcttcagattgcaaaccttcttgccttcaagcctcggctccaacaccagtccggcagaggaacccagtctaatgaggtacgctcccttcctgccattctctattccattaacctgtttcgtggtaaacgtaggactgatcctccaaaattaccttattaattagcttacatatttattatctatctgtcccaccagaatgcaggtttccggaaggcagggatttaaaaaaatctgttttgttctatgtgattttcccataccaagcaccgtgcccggcacaagctgggatcccagtacacatctcgggacggaagaaccgtgtttccctagaacccagtcagagggcagcttagcaatgtgtcacaggtggggcgcccgcgttccgggcggacgcactggctccccggccggcgtgggtgtggggcgagtgggtgtgtgcggggtgtgcgcggtagagcgcgccagcgagcccggagcgcggagctgggaggagcagcgagcgccgcgcagaacccgcagcgccggcctggcagggcagctcggaggtgggtgggccgcgccgccagcccgcttgcagggtccccattggccgcctgccggccgccctccgcccaaaaggcggcaaggagccgagaggctgcttcggagtgtgaggaggacagccggaccgagccaacgccggggactttgttccctccgcggaggggactcggcaactcgcagcggcagggtctggggccggcgcctgggagggatctgcgccccccactcactccctagctgtgttcccgccgcgccccggctagtctccggcgctggcgcctatggtcggcctccgacagcgctccggagggaccgggggagctcccaggcgcccgggtgagtagccaggcgcggctccccggtccccccgacccccggcgccagcttttgctttcccagccagggcgcggtggggtttgtccgggcagtgcctcgagcaactgggaaggccaaggcggagggaaacttggcttcggggagaagtgcgatcgcagccgggaggcttccccagccccgcgggccgggtgagaacaggtggcgccggcccgaccaggcgctttgtgtcggggcgcgaggatctggagcgaactgctgcgcctcggtgggccgc

SEQ ID NO: 44 - DNA, 2501 bp, Homo Sapiens
gatgtgaaaagagaaataattgaaaaagactggagtacatatactatctacagtgtctgttttaaagaaacaacattctagcacacctttctacccttgactaagattactgtaatgagagcaccagtacccctgagtaaccgaaagggcattttggaaactgagcttttggtgtttatatgaacattctgtcttccaggacctgccttgatttattcaagactcatactgctgtatatggtgttgtatacattaggggtagttgggtagcagtaactgatatagaaaattttaaatgtaaaaaacactggggagtgaacctttccattatatatatatatatatatatatatatatatatatatatatatatatatatatataaattcacatcaggatgagtttctgtttaggcaatgttggaaaacgctatttccattttttttttttaacaaatatttaacaaacatttataaggcacttaaatccatgctggctcttacaaatgttgactcatttctcataaccaccttggggtagaaacggagaggctaaacaatctgcaggcgatgcttcactactaaatgcaggtggcagccttgcctgtgttctctgcttggctaggaacacaggtcttacctattgagctgggctgtgtagaactctgttgtggagacatctgcccctggggcagaagcctctgctttttccccctcctcccatcttactccatgtctcagagagctctgaatcccacttggagaatcacacttaaaccctctaaaaacctaatgatgaataaaaataagttctctagaacttctggagaaaaaagtaataaagctaccaggttaaatgactgaaattcctgagagaaaacaacatgtgtgtgtttctctagaaagggggcccaatactgaataccaggaagtcctatagtaaatggaatgtgactctatgtgggatccggcgttcctatttcatccgaatgcatgtctgctgcttcagtgggaagggtgcttgcacaccaggtacccactccctggtgtcatgtgctatgcagtccaaagacagaaccaggaatggtgagcccatgagcctgctggacccagcccctccgaggtccggagtgacaaccagtgccgtatttctagatcaaacctgaacccctcctacagggaaaagatttccaggggattttgaaagttccaacattttacagggaagaaggaagataagcaggatatgaaagaagagttcatgttatacagccctggcttccactgacgctaacactggattcagcttttgacactgataatctgttgccaccaaatggaaaacgtaaacaagatattctaagtgtggttagagaatatgcaacacaaggaacaagcagaacattcttctctggaatctgacataatggactgtactttcacagacagcactgatgttagatgtacgtgaaataggctaaactgaaaataagaaaggctgaggcagagaggataatatagctccagcctatctcccagcaccttgttaatttctctcaacctccagccacaaatccgagacacaacgctcttcctccaaagaggtcgcgccttctctgtggtggttctcagggatccgccccagctccttctccgttcccagccccacacactgggatcaccaggcacccaagatcccacctctcaggtggtatcttcagcgcaggctgccactcagcccccctccagggatctggggcagaaggcgaatatcccagagtctcagagtccacaggagttactctgaagggcgaggcgcgggctgcatcagtggacccccacaccccacccgcaccccaagcgctccaccctgggggcggggccgtcgccttccttccggactcgggatcgatctggaactccgggaatttccctggcccgggggctccgggctttccagccccaaccatgcataaaaggggttcgcggatctcggagagccacagagcccgggccgcaggcacctcctcgccagctcttccgctcctctcacagccgccagacccgcctgctgagccccatggcccgcgctgctctctccgccgcccccagcaatccccggctcctgcgagtggcgctgctgctcctgctcctggtagccgctggccggcgcgcagcaggtgggtaccggcgccctggggtccccgggccggacgcggctggggtaggcacccagcgccgacagcctcgctcagtcagtgagtctcttcttccctaggagcgtccgtggccactgaactgcgctgccagtgcttgcagaccctgcagggaattcaccccaagaacatccaaagtgtgaacgtgaagtcccccggaccccactgcgcccaaaccgaagtcatgtaagtcccgccccgcgctgcctctgccaccgccggggtcccagaccctcctgctgccccaaccctgtccccagcccgacctcctgcctcacgagattcc

SEQ ID NO: 45 - DNA, 2501 bp, Homo Sapiens
ggcgacagagcaagactccctcttaaaaaaaaaaaaaaaaaaagattctgagtcaaagtgctcaagttgaatgcattttgtcatccacaagacaaatcgtgttaaccccttgtggtttactttatctataaaatagagataacaatagttcctgcttctagggttgttgtgggaattaaagacttagaataatgttcagcctctaatcagtgctgtcacaactgtctgatacaattgtattatatttgtgtactttgtagattgatattaaatcatacttttaaaaataggtgcttaatgttccactcaattaccttaaaacatgtttaattatgtctctatcctactcttataacacttctataaaaactttttacatatagcgtccacttttggttcagtttcttaggaaaataactttgagagtcagctatctgaaccaaagaaacattaacattaccagactatattgggatttttgagactggcttttatcaattctttagctacgggctcttgtcatcatctctaccagtgacctaagtgtcaaacccaaatgccttgtatctgtcccattaaagagatgcagcatctgctcctttcttactgtttccatttcctctgccatgcctcctcttacaaccataaatatccaggtctcttaggttttaaacggggcatctctcaacccccacattcttttccttggttattcccttccctccaacagttcaattcacctagatccccacgcctgaaattatcctagatgtcctagaggcgcctcatcattacaatggtacattattctccactcctttacatgtcacgccagctttcaaactgaaaatctgagcgttcatccctggtgcatcacctttaaattccagatctccaaaatccagggtcatgtaaccttaaaaaatttttaccctctcttctccactgcccttgttcaggccttatctcttccagcagctgttccaaaggcctactctgttttcctttcggagtgctaacctccaccgaagcctccacccagttgccaattctgccccatgcctgataatttgctcgtgcgttgacatacataaaatttctaagacaaaaattttttaataatggtaaatgaaccttgggaactgcatacagatcatacagatccataataagagaaaaggtcccagattaacacggaaaactttccatttaactaacatttgcactggtaaacttcatcaagcaagaccctacttaatcccacattaccttctactgaagaggttgtggtcattctctggaaatatctgaattcattcctacaagttagagaaacagcgttactcgaaacattatcccttgggctcgagctctaaggcacctgacaaacggagcgctgtgggtaggggtgaggtgttttctccagggctgggactttgccctgggcgagggcgccgcagggcaaagacctcaccgggcagcagaatccgggcagaaatcagcaactgggcctcccgcgcagcagaaaaggggaatccagtcggggcccacccttcctgccagcgcagaccgcaagtctggccccatcctctcgccgggagtcggcctggcgcgtcccgcccaggtaccccgaccgtgggcagcctgcgcccgtttgggtcccatcgccccggcccggcagatacctgagcggtggccagggcaggtccccgttcttgccgatgcccatgttctgggacacagcgacgatgcagtttagcgaaccaaccatgacagcagcgggaggacctccgagcccgctcgttacagcagaacgcgcggtcaagtttggcgcgaaattgtggccgccccgccccctcgtccctatttgtgcaggcgaggccccgcccccccgccccggcgcacgcagggtcgcggcgtgctcgcgcccgcagacgcctgggaactgcggccgcgggttcgcgctcctcgccgggccctgccgccgggctgccatccttgccctgccatgtctcgccggaagcctgcgtcgggcggcctcgctgcctccagctcagcccctgcgaggcaagcggttttgagccgattcttccagtctacgggaagcctgaaatccacctcctcctccacaggtgcagccgaccaggtggaccctggcgctgcagcggccgcagcgcccccagcgcccgccttcccgccccagctgccgccgcacgtagtaggttctgtctgggactgggcagggccatcggggctgggggggcggggcttgtgggtaaggcgggcggaggcgtggaccctccgcccgatgatagggctggaggaggaaggggcgggctgaagaaggggaaggtgggaagagcccagccggggctacaaattgggtgaagcgctgaggttttagtacttccgtttgaggagataggcaaaggttatgcaggtttttaatggcaggcctgagacaggaactcaggtctcctgactcccattctgatgagggg

SEQ ID NO: 46 - DNA, 1092 bp, Homo Sapiens
aagcttccccttcatcatccaagaaggcattcaggtctttctgtgctaggccccaggtaaagtgctggactacccagtaattgggttcagtagcaggatggcctcagattgaggtcccagggccaaaggaccactcctctcctcagcgctggtccgggaaaggcaagctccgggcgggagcgcacgccgcgcccccgaagcctggctccctcgccacgcccacttcctgcccccatcccgcgcctttccaggtcttctcccggtgaaccggatgctctgtcagtctcctactctgcgtcctcggccgcggcccgggtccctcgcaaagccgctgccatcccggagggcccagccagcgggctcccggaggctggccgggcaggcgtggtgcgcggtaggagctgggcgcgcacggctaccgcgcgtggaggagacactgccctgccgcgatgggggcccggggcgctccttcacgccgtaggcaagcggggcggcggctgcggtacctgcccaccgggagctttcccttccttctcctgctgctgctgctctgcatccagctcgggggaggacagaagaaaaaggaggtagaatggatccccttggccttcccctgtggtcgggggcgggccagggtgggccgcgttgcccaggcagccctgccgtgttgctaggcagcctggtcgccggcgtgggcgatgccggcgctggggcgggagccgcgagggtgggaggccctggggcgtttccgggacgtggagttagcagggttctgacttgaaaaacgacggcaaagcgtgttcttgactgcttctgagcacctcacacctttcagacccagggcgcctttattcccagctggaagcccagcttagagcaatggtgccactaaaaggggtgtgttggatgtgaaaataccctttggaagtatttataagcctgcaggaaatatgttttccttattttcttactctgctcccttcattacccatttcaagaagcaacagaacctgtgcagagtgtgttttaagttacactgtatgtttatttttgtttatgttgaactcggtgtatacttgtgagaataagctt

SEQ ID NO: 47 - DNA, 2501 bp, Homo Sapiens
cgaaatgaaacctcgcccaggaggccgcggacctggacacccggcgccacctccttcacctctgacccaggtttcctcccggcgctgcgagctcccggggaagggttagagccggcagccctccccagcccggggaggggagagggttatgcgaccccacctctggctagggccggggaggcctttgcttcccgggagccctgcccgggctccttggtcgcagggctgctgggtcccaggcaggaacgagagggtgaggcccacatgtggcccggcggcccagggcggcttgcagcgtcctcactgtcccggctgccaggggctgcggcgacgcggccagtcagcagcgagttcaggtcgcgcagattttattgatgagctctgactttcagcactttccctaagtcaagaagagtctagcgtacccttcggctgcttcatttcagcctccctgcctcagctcttcagccctattccccctcgccctgtcctggggtgtgtacagcagcccaggccttccttctccttcccggctccgtggcccgaagccgccgagagagctcgggacagcgcaggaccaggcagccgctcgctctcctgtcaccttaactgcaggctccgaggggcgcctttggagtgtactgaggtgtgtcctaatcgtgcggcattcaacaaatggacttctggtgtgtggtcagaagagaaaagccatttacttactttcctccccggttttctggcaacagctgaaggggagctgcctccgtggactgagcagacccaggagagggagtcgtggtgcggagacacacgcaccacacacagatgaccggtggcacacacgacacacgctgacataccgacatcgccagtgggacacacacacacacacacacacacacacacacacacacagagagagagagagaatccctcccagcattggtcatccgcccccccacccaggcttccactccccctcccctcttatctcccctggcttcccctcctctcgggcgctgcgaaaagcagccgcacttagtcaacaaatggcacgtgggagaagttggtgagtgtttggtgaggactcttcagggcttttcacaagaaccctctgtacacaaagtaagtggcgtgtttactcgggcctctccagccagagctgtgcctctgctccgctgcgcaccgcggcttccgaaaggagaaaggagagaagaaagggcggggagagcggggtggaggatttggacaggccctggaggcttgggctggggaggcctctggcctcgtttagttctcggcccggcaacctcctctcggcctaggcttcgccgcggcctccgcagctggaatggagctgccaggacccagtgacgctcccgcccctttcctcttcttccaaggggccaggtgggctggggtgcggccgccgctgtgctctgtgtcttggggccccggctgggatggggtgggggcgggcgggggcggggcggcaggccacgctgtcctggagttggcaagaaaggacagcacagaaacttgcaccctccgaggactgggagtcccgagtccagcttagggggagtgggggcgcgacccccaacccagaaaccttcacttgaccgctcaagttcgcggcagcagggcgggccgcgccgaatctcggcgtgcgcggagcggggagatgcaggcgagcgccagagcccgggctcgggggccctgcgccggggagaggagccgggacccaccggcggagccgaaaacaagtgtattcatattcaaacaaacggaccaattgcaccaggcggggagagggagcatccaatcggctggcgcgaggccccggcgctgctttgcataaagcaatattttgtgtgagagcgagcggtgcatttgcatgttgcggagtgattagtgggtttgaaaagggaaccgtggctcggcctcatttcccgctctggttcaggcgcaggaggaagtgttttgctggaggatgatgacagaggtcaggcttcgctaatgggccagtgaggagcggtggaggcgaggccgggcgccggcacacacacattaacacacttgagccatcaccaatcagcataggtgtgctggctgcagccacttccctcacccacactctttatctctcactctccagccgctgacagcccattttattgtcaatctctgtctccttcccaggaatctgagaattgctctcacacaccaacccagcaacatccgtggagaaaactctcaccagcaactcctttaaaacaccgtcatttcaaaccattgtggtcttcaagcaacaacagcagcacaaaaaaccccaaccaaacaaaactcttgacagaagctgtgacaaccagaaaggatgcctcataaaggtgagtccgcttctttcttctcgctttatttttattgcaatattcagacaggtctcccccttcctccccccttccttcctcccctctcgccggtcccctcccccactgcta

SEQ ID NO: 48 - DNA, 2501 bp, Homo Sapiens
tgatggttgcacaactctgagtacatgaaaaatcaatgaactgatactttgagtgagctgtatgatactggaattacacctcaataaagcatggtaactgttttaagataggctggaaagagaaagcctgaaaacaacaataatgatattaataaattagtttacttctctagtctcatatacttctgtgcccacacttgctcctgttctattcataatggtccccttgcagttgccatattatatcctgccatttgatgcccggtgaacattctatacctgcttcccagaattctctttacctttcctctatctgcctaacttccacatatctaaaattaatcagagtaaactatttactagaacaaccaactccaaatcctagtaacctaacatgataaaggtttgtttctcactcatatagcccctccccagatgatcgaggggtccaggctccttacctctagtggctcccccaccttctggagtcttctgcattctttatacatggttgagataaactatgagtcattagcacagctagaccttgaggtcctacaagaaaatttgcaaatcattcactctgttttgaacaaggtatatttaagatgatgttaaaatacccaatggtcttgggtcaaatacagtttatgactgtgtatctaaaatatatattgcaatattcttccctttttctactgacttcatgaatttagcggggatccattttataagctcaaagataattacttttcagactaagaatatttagggtaaaaagtactgttcaacatctctactgaggatgttatgatgtagcacactgtataagctggagctaaaggaaactttccttaaagtgctatttactaaaaattggaacacattccttaagacaaatcgaagtgtggcacacaacatccaaacttccatcatagatacagaggtgttaccatctcccactcccaaatttctttgtcacgctgaggatactcaagaggagcaggacatgttggtcgcagcaggagaaacttgaaagcattcacttttatggaactcataagggagagaatttcttattttagtatcgtccttgatacatttattattttaaaagataatgtagccaaatgtcttcctctgtgttaaatctttacaaaactgaaatcttaaaatggtgacaaaaattctacttctgatagaatctattcatttttccaattagatagggcataattcttaatttgcaaaacaaaacgtaatatgcttatgaggttccatcccaaagaacctgctattgagagtagcattcagaataacgggtggaaatgccaactccagagtttcagatcctaccggtaattggggtagggaggggctttgggcggggcctccctagaggaggaggcgttgttagaaagctgtctggccagtccacagctgtcactaatcggggtaagccttgttgtatttgtgcgtgtgggtggcattctcaatgagaactagcttcacttgtcatttgagtgaaatctacaacccgaggcggctagtgctcccgcactactgggatctgagatcttcggagatgactgtcgcccgcagtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcgggagataaaggagccgcgtgtcactaaattgccgtcgcagccgcagccactcaagtgccggacttgtgagtactctgcgtctccagtcctcggacagaagttggagaactctcttggagaactccccgagttaggagacgagatctcctaacaattactactttttcttgcgctccccacttgccgctcgctgggacaaacgacagccacagttcccctgacgacaggatggaggccaagggcaggagctgaccagcgccgccctcccccgcccccgacccaggaggtggagatccctccggtccagccacattcaacacccactttctcctccctctgcccctatattcccgaaaccccctcctccttcccttttccctcctccctggagacgggggaggagaaaaggggagtccagtcgtcatgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcgg

SEQ ID NO: 49 - DNA, 2501 bp, Homo Sapiens
taccttcataaaaggatctttgacttggtaagtgtgtgcgatgcatacttttcatgttacaccacaagtgccacttagcaactccactagacagggcagtgtttcagcatggggtggggtgccccctgacaggcttttaaaaggcccggatgccaatgcacattccaacactatccacaaaaaggagactggagcagtgctcttccctgcattgggcaaggagactctccctccctgcctaaccacttgcctgccctgttttgtgggagaattacaagtaaatgctacagaggcagtggagaaaaaagggtgttttaattcctctccagagtttcctttatttgatgtatgttgcatcctttaaacaagttgtgcaaaatggctgcagggtagattggctctcccttttaaagctctccatccggctgggtttatttgtaaatactgcatctatccttcttagtgttttaggactggctggaaagactcttcttcctgtaggttgggtcagtgtgagagatctaaaaaatcattttcccttaaaattactgtattttaataaaaggattgggcaggggctggaatgagagaaaactggtccttcaaaatgtaaaactgtcatacttaaaccagtttacaaaatatgcgtttaattatgtggtgggatgtgtgtaggtgtatgatgagagaggcaaccaacatggctatttggggtgcaaggatgtgggaacaggcaagtaattttcacattggactttcatcctagggagctgggttctagtcacagctctgagctgtgtgaccttgggtaggtctcatctccccggggttttgtttcaccagttgaacagtatgaggatgagtcacagctaacatttgttccatgatatttacccagcaccatacaagtgttatttctgtcctcccagttaacactgacgtgggtagtattatatgcccattttacagatgaggaaactgaagcctgaagaagttaaatacttatcccagaacacacagctggtaagtggcagacctggaattggaatctagttcagtttgattccccaacccatgctcttgaccactatactgttttttcaagtccagatctgaaatctcattttctgtgtggctgtgtgtttgggacaggggtaaccaattcctgactactctatatgctgcatagaacctggagaggatttttcaaagtaaatgaatctcgaaagctggattgcagagcaaacgagtgcagtcaattcagccaggggcttgcaagagggagaaagagaaaaagactgtggaatggaaagtttcccaacccaagcctttcccaaggggtagccattctctgttctacagtttagggcttgcatgtgctttttctggagtggaaaaatacataagttataaggaatttaacagacagaaaggcgcacagaggaatttaaagtgtgggctggggggcgaggcggtgggcgggaggcgagcgggcgcaggcggaacaccgttttccaagctaagccgccgcaaataaaaaggcgtaaagggagagaagttggtgctcaacgtgagccaggagcagcgtcccggctcctcccctgctcattttaaaagcacttcttgtattgtttttaaggtgagaaataggaaagaaaacgccggcttgtgcgctcgctgcctgcctctctggctgtctgcttttgcagggctgctgggagtttttaagctctgtgagaatcctgggagttggtgatgtcagactagttgggtcatttgaaggttagcagcccgggtagggttcaccgaaagttcactcgcatatattaggcaattcaatctttcattctgtgtgacagaagtagtaggaagtgagctgttcagaggcaggagggtctattctttgccaaaggggggaccagaattcccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatggtaaatgatcatttggatcaattacaggcttttagctggcttgtctgtcataattcatgattcggggctgggaaaaagaccaacagcctacgtgccaaaaaaggggcagagtttgatggagttgggtggacttttctatgccatttgcctccacacctagaggataagcacttttgcagacattcagtgcaagggagatcatgtttgactgtatggatgttctgtcagtgagtcctgggcaaatcctggatttctacactgcgagtccgtcttcctgcatgctccaggagaaagctctcaaagcatgcttcagtggattgacccaaaccgaatggcagcatcggcacactgctcaatgtaggtttatttttttcccttct

SEQ ID NO: 50 - DNA, 2501 bp, Homo Sapiens
ggaggatagaaatataaattaaagaatgacacaaataattataaagttacagctgttaaaagaaaagcatatggtgccaagagaacgtgtaatacaagatctactcatggaggtgagggaaagcttgcccatcaaagaagttatgattcaatccacgaagaccaggagttggctgggtgaagaaaaaaaggtcagaggaaggaagtccacactggggaaggctctaagcataaagggtaggaggattacagaggcatattcacgaaatttggagaaggctttcagtaagcaaggagaagccaaatgaaagtttacgggagagttggaggcttgaagacacgttcaaggatctggtttttatcttctctttatctcaagagcagtgggaagccattaaatgattttaatcagagggttggtataactagttttgtattttgaaaagctgaattcagctctcgtttgagaaactgagtgaaagagcccagaacggccgtggctgagggtgactcgtgggagactcctacacaagccatggcagtggcatgggctggtggcagaagagggaatagggagaagatttggaactcaatcttcctccattgacaaagtcactccagctttggcaaggcaattaattggtgggaaagaagatgcctagccctcctgatttcactgcactttctgcatcttcaacatgagtactgggaagtggcaaaacatccagaggcagcttgggtgctaggtggagcatgagttaaaattccaggatgaagcaaatgaacacttagaatgacaggaaagatttgggagttgggtttgggggagggctatttacctttattccctggagaccctggcacaaacccttgcctctgcaatcttcctctcaggtaaaggaattcattaaatgaattgctagaagatctactgaccagagggctgtacagaatcatatctttgagagtgggaagtaggttgatcacatagtttattatccaatcaggacatatctgaaagagaaagggggttctattaatatttaaactacaaaacatgtacaccaggaatgtcttgggcaaatctggttgccctagcaagaaaggaaatttgaaagtttatactgttctgctcccatgttaccccgtttgcacatgagagggtaagtattctctttcttcacctgcattaagggaataaaagcacaagcattcaggtgactcccaacccacttttaattttacagtttctgctatactctatacattctgaaaattacatttcccaccactatcacttcgtgataggtgatcatttacaattactcactgactcagtcccgggaagaggcggtgcaaaatgggacgctctatccaggtgctcattagaaatgcagaatctctgcctgcctcctagacctactgaattagaatctgcatttttaaataagatttccaggtgatcaatatgtacattaaaacttgagaaaaacctctagacttcgacctaaagaaaaacattttacaacttgacagtgtatgcacatacatacatgcatatagacacaactgaagcacaaatttaatgaagtagaatttaccgttactattttatttgggaaagaaatgtgctcgcgactcaatagattggagtattcactcctggatctcaacttgcaatttgaaaacgcatctctaaagcacctaggagcaatctgaagaaagctgaggggaggcggcagatgttctgatctactagggaaaacgtggacgttttctgttgttactttgtgaactgtgtgcacttagtcattcttgagtaaatacttggagcgaggaactcctgagtggtgtgggagggcggtgaggggcagctgaaagtcggccaaagctctcggaggggctggtctaggaaacatgattggcagctacgagagagctaggggctggacgtcgaggagagggagaaggctctcgggcggagagaggtcctgcccagctgttggcgaggagtttcctgtttcccccgcagcgctgagttgaagttgagtgagtcactcgcgcgcacggagcgacgacacccccgcgcgtgcacccgctcgggacaggagccggactcctgtgcagcttccctcggccgccgggggcctccccgcgcctcgccggcctccaggccccctcctggctggcgagcgggcgccacatctggcccgcacatctgcgctgccggcccggcgcggggtccggagagggcgcggcgcggaggcgcagccaggggtccgggaaggcgccgtccgctgcgctgggggctcggtctatgacgagcagcggggtctgccatgggtcgggggctgctcaggggcctgtggccgctgcacatcgtcctgtggacgcgtatcgccagcacgatcccaccgcacgttcagaagtcgggtgagtggtccccagcccgggctcggcggggcgccgggggtcttcctggggtccccgcctctccgctgc

SEQ ID NO: 51 - DNA, 2500 bp, Homo Sapiens
ttcccatcaagccctagggctcctcgtggctgctgggagttgtagtctgaacgcttctatcttggcgagaagcgcctacgctccccctaccgagtcccgcggtaattcttaaagcacctgcaccgcccccccgccgcctgcagagggcgcagcaggtcttgcacctcttctgcatctcattctccaggcttcagacctgtctccctcattcaaaaaatatttattatcgagctcttacttgctacccagcactgatataggcactcaggaatacaacaatgaataagatagtagaaaaattctatatcctcataaggcttacgtttccatgtactgaaagcaatgaacaaataaatcttatcagagtgataagggttgtgaaggagattaaataagatggtgtgatataaagtatctgggagaaaacgttagggtgtgatattacggaaagccttcctaaaaaatgacattttaactgatgagaagaaaggatccagctgagagcaaacgcaaaagctttcttccttccacccttcatatttgacacaatgcaggattcctccaaaatgatttccaccaattctgccctcacagctctggcttgcagaattttccaccccaaaatgttagtatctacggcaccaggtcggcgagaatcctgactctgcaccctcctccccaactccatttcctttgcttcctccggcaggcggattacttgcccttacttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgggtaagctcctgactgaacttgatgagtcctctctgagtcacgggctctcggctccgtgtattttcagctcgggaaaatcgctggggctgggggtggggcagtggggacttagcgagtttgggggtgagtgggatggaagcttggctagagggatcatcataggagttgcattgttgggagacctgggtgtagatgatggggatgttaggaccatccgaactcaaagttgaacgcctaggcagaggagtggagctttggggaaccttgagccggcctaaagcgtacttctttgcacatccacccggtgctgggcgtagggaatccctgaaataaaagatgcacaaagcattgaggtctgagacttttggatctcgaaacattgagaactcatagctgtatattttagagcccatggcatcctagtgaaaactggggctccattccgaaatgatcatttgggggtgatccggggagcccaagctgctaaggtcccacaacttccggacctttgtccttcctggagcgatctttccaggcagcccccggctccgctagatggagaaaatccaattgaaggctgtcagtcgtggaagtgagaagtgctaaaccaggggtttgcccgccaggccgaggaggaccgtcgcaatctgagaggcccggcagccctgttattgtttggctccacatttacatttctgcctcttgcagcagcatttccggtttctttttgccggagcagctcactattcacccgatgagaggggaggagagagagagaaaatgtcctttaggccggttcctcttacttggcagagggaggctgctattctccgcctgcatttctttttctggattacttagttatggcctttgcaaaggcaggggtatttgttttgatgcaaacctcaatccctccccttctttgaatggtgtgccccaccccccgggtcgcctgcaacctaggcggacgctaccatggcgtagacagggagggaaagaagtgtgcagaaggcaagcccggaggcactttcaagaatgagcatatctcatcttcccggagaaaaaaaaaaaagaatggtacgtctgagaatgaaattttgaaagagtgcaatgatgggtcgtttgataatttgtcgggaaaaacaatctacctgttatctagctttgggctaggccattccagttccagacgcaggctgaacgtcgtgaagcggaaggggcgggcccgcaggcgtccgtgtggtcctccgtgcagccctcggcccgagccggttcttcctggtaggaggcggaactcgaattcatttctcccgctgccccatctcttagctcgcggttgtttcattccgcagtttcttcccatgcacctgccgcgtaccggccactttgtgccgtacttacgtcatctttttcctaaatcgaggtggcatttacacacagcgccagtgcacacagcaagtgcacaggaagatgagttttggcccctaaccgctccgtgatgcctaccaagtcacagacccttttcatcgtcccagaaacgtttcatcacgt

SEQ ID NO: 52 - DNA, 286 bp, Homo Sapiens
tttgcactaggctggaagtggccgccagtcccccgtgcaattccattctctggaaaagtggaatcagctggcattgcccagcgtgatttgtgaggctgagccccaacagtccaaagaagcaaatgggatgccacctccgcggggctcgctcctcgcgaggtgctcaccccgtatctgccatgcaaaacgagggagcgttaggaaggaatccgtcttgtaaagccattggtcctggtcatcagcctctacccaatgctttcgtgatgctgctgctgatctatttggg

SEQ ID NO: 53 - DNA, 1400 bp, Homo Sapiens
  unsure (1371): unknown base
ttccagctgtcaaaatctcccttccatctaattaattcctcatccaactatgttccaaaacgagaatagaaaattagccccaataagcccaggcaactgaaaagtaaatgctatgttgtactttgatccatggtcacaactcataatcttggaaaagtggacagaaaagacaaaagagtgaactttaaaactcgaatttattttaccagtatctcctatgaagggctagtaaccaaaataatccacgcatcagggagagaaatgccttaaggcatacgttttggacatttagcgtccctgcaaattctggccatcgccgcttcctttgtccatcagaaggcaggaaactttatattggtgacccgtggagctcacattaactatttacagggtaactgcttaggaccagtattatgaggagaatttacctttcccgcctctctttccaagaaacaaggagggggtgaaggtacggagaacagtatttcttctgttgaaagcaacttagctacaaagataaattacagctatgtacactgaaggtagctatttcattccacaaaataagagttttttaaaaagctatgtatgtatgtgctgcatatagagcagatatacagcctattaagcgtcgtcactaaaacataaaacatgtcagcctttcttaaccttactcgccccagtctgtcccgacgtgacttcctcgaccctctaaagacgtacagaccagacacggcggcggcggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaatttgtggggggtacgaaaaggcggaaagaaacagtcatttcgtcacatgggcttggttttcagtcttataaaaaggaaggttctctcggttagcgaccaattgtcatacgacttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctgncgctcagccatacaggtgagtacctggcg

SEQ ID NO: 54 - DNA, 2501 bp, Homo Sapiens
gataatcttttcatacaagatgcattctgcttttgtgggcctcttgcagccctcaagcccccatctgatttgtacacaatgatccagtgggccagaggagcccagagccatgagcggcccatccctccaagaactatttctgactgtccagtatcatggagcaagtggaaagaagaaaaaaaaaacccaattacttttcgaagagcaagatgaatgctgtagaaggagaaggaaggggagggagatggatgggtgccgattccagaatcttcagatctgcttggatgaatcattacctatgatttgcgggacaagaatctgattttattcatcaaccagtagaaacttttctttctgcctcccaacatctgaaatccaacaaacatgtgccttaggaacataccggtcatcttttagaggcattttatatacatattgagtaactagaaaacactctttccgtaatacacacacacacacacacacacacacaccatcttgtcatacaacactcccacgcaagaaaagcgaaactgctgtttgatgaatgtaaacacttggctgtttgcagcagtcgggagtcctgccaggtttaagtgctaagatgggaggtgaaccccaggggtttccccctgcccgtgctgagatccttatttggtcaagcttctacctatgccctggcctcggagcgagcccgatagcgctggatcacagcagagggagcgaggcggctgacgtcccatcccgaagagatgaatggaattccaggaagctagagtcatgctggcttgggacagtggcttggagaccagacttcaatgacagaagcactaggcagcggcactcatggcaatgtgtgcacccacagaaatgtaacccacacctcgggttcaggagccgaaaaatgaaaagaacgtttagggaggaaaaagggaaatacaataataggcagagagtaatttattactctatgggtctgctctgtaaatagctgaagactctggagccagatggttctgcaaattctccaaacaggagtcacgttaagaagcacgagtgggcacaaaaactgtttttcaagacacaatttcaatttggcttgtggaaactggatacgagtaagtttccttaaaattcgagtagaaagcagctgtcctccccgggccccttgatgagaatacgcacaccgcccccaagcggccggccgagggagcgccgcggcagcgggagaggcgtctctgtgggccccctggcagccgcggcaggaaagggcccgaaggcagcgaaggcgaacgcggcgcaccaacctgccggccccgccgacgccgcgctcacctccctccggggcgggcgtggggccagctcaggacaggcgctcgggggacgcgtgtcctcaccccacggggacggtggaggagagtcagcgagggcccgaggggcaggtactttaacgaatggctctcttggtgtcccctgcgccccgtcggcccatttttctttttacaaaacgggcccagtctctagtatccacctctcgccatcaaccaggcattccgggagatcagctcgcccgaaagcccctgcgccaccccgcgggccctcctaggtggtctccccagccccgtcccttttcgggatgcttgctgatcaccccgagcccgcgtggcgcaagagtacgagcgccgagcccgtgcgcgccaaggctgcgtgggcgggcaccgacttttctgagaagttctagtgctcccaagccccgacccccgcccccttcactttctagctggaaagttgcgcgccaggcagcggggggcggagagaggagcccagactggcccccacctcccgcttcctgcccggccgccgcccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggtaagtcgcccccggcggccgccgaggaccaaagctgcccgggacatccacctggagcgctgaggcttcagtccctctggtggaccccggaacctacactctccccgctcgcctaccccagcccgctcctctcagccgctggaggactcttcagggcaaggctccagagccatcctctccagccttgaggttcacaaaccaactcatcaggacaccccaagatttccttactctctgaagtcctccttaagcctttgtatcagcactccagggaagagtctgtacttcccctgccctccctgcaaccccaaactacagttcctgatcttgctcaccttcgacttcccaaaagcccccaaattgttggtcttgcgccccccacactttaaaaccagcatctctttcctccacctctctct

SEQ ID NO: 55 - DNA, 7258 bp, Homo Sapiens
ttcaataggaagcaccaacagtttatgccctaggactttgttcccacaatcctgtaacatcatatcacgacacctaacccaatccttatcaagccctgtcaaaaacggactttaaaccaagctgcaaattttcagtaatctggccttgcctttccccctctgatagcaccatcaaacaaacccccttactgccgaaagcaataagcccggctttgttccatccactggttgtgttggtgatatctggggactgccactgaacagacgcacagagggagcccctacaggcaggggtttttctgtctgtgcttcttgggagagtatgtctcgtacatttgtcgcgtgatgaagacttcacagctccatccagcgaccagactcacagctccatccagctgcggcaagggggtctgaggcagtcttaggcaagttggggcccagcgggagaagttgcagaagaactgattagaggacccaggaggcttcagagctgggctgaggtagagagtctcctgtgcgccttctctcctctctgcaattcggggactccttgcactggggcaggccccggcaggtgcatgggaggaagcacggagaatttacaagcctctcgattcctcagtccagacgctgttgggtcccctccgctggagatcgcgcttcccccaaatctttgtgagcgttgcggaagcacgcggggtccgggtcgctgagcgctgcaagacaggggagggagccgggcgggagagggaggggcggcgccggggcgggccctgatatagagcaggcgccgcgggtcgcagcacagtcggagaccgcagcccggagcccgggccagggtccacctgtccccgcagcgccggctcgcgccctcctgccgcagccaccggtgagtgccgcggtcctgagatccccgggccggatgcgcggcggccccagctcccgagcgtctgcctgccccgccctgggctgcccgggctccctgggctccccggcggctgcacggagtcaaggcgccccgtcccgggcgtcccccgcgggtgccgatccaggctgcccggagtccggagcccatagaggagagagacagctggggagcctggtcaccgcgggcatctcccctgcgctgcagtcgcccgcctggcctgccttcccgttcctccgcctcttgccctgacttctccttcctttgcagagccgccgtctagcgccccgacctcgccaccatgagagccctgctggcgcgcctgcttctctgcgtcctggtcgtgagcgactccaaagtgagtgcgctcttgctttgactgatgctgcccaaggacctctgatcagcaccaggggagaggaggggctgctcagggagctggggtctccggattccatccacagcagggccagactctccccaggaaatgggacagggtggcagcggaggcttgagaaccacgggggttggcactggctggcaagggaggaagagggccaccgggactgccccagcctgcgggcatctggtagatgaagcttaatccatttctcctggctggaaaccatggtcttccatttgagaactagatacgaacagggtgaggcgagagggagagggaagagtgggttttgggattggggccagtttaccctcaccctggatccctggagcatgggacctttgatgaagcctcctcccgaatctcttccagggcagcaatgaacttcatcaagttccatgtgagtatccacccctacaacagttggctgcacagacaagttgggaaggcttcaggggacactcccctccctgccctctgctgcagcgtgcgccaccccttaccacttccactccccctcgcttaccccacctttgttctctccagcgaactgtgactgtctaaatggaggaacatgtgtgtccaacaagtacttctccaacattcactggtgcaactgcccaaagaaattcggagggcagcactgtgaaataggtatggggatctccactgcaactgggagagaaatttggggacagggagggatgggtgggaggcaagagcaggcaggagttaggagctggaggtagggtgggtgacatcttcatccctatgtgacaagcataaacacacacacacgctcacgaaacagtggccacacaaatgtgaggtggggttggaaggagaccctgtccagtcttctggcaggtctgaaacgacatctttaaaatgtccgttggcagccgggcatggtggctcacgcttgtaatcccagcattttgagaggtcaagtttgagtggatcatttaggtcaggagttcaagaccagcctggacaacatggtgtaaccctgcctctactaaaaatgcaaaaatcagcctggcatggtggtggatgcctgtagtcccagctacttgggaggctgaggcaggagaattgcttgaacatgggaggccagatctcagtgagctgagatcacaccactgcactccaactgggcgacagagcaagactccatctcaaaaaaaaaaaaaaataaaagttagttggaatgttcttctctttctcatattctctcatcctcctgtccccttgtagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggtgaggtgggggcaacaaggaccaaaagccctccctacagcttcccagaaaccttgttaccatccccttctcccagagggctggccatagcacaagagaagtgcggcctctggttgagtcttccctgaggggaggaggcagggaaggccctctgggttggaatgacatcccctatctttctgtgttgtgccaggaacccagacaaccggaggcgaccctggtgctatgtgcaggtgggcctaaagccgcttgtccaagagtgcatggtgcatgactgcgcagatggtgagcatcactgacctgctgatgacaggtgggtggaaggggacaaacttacatgtccccttattccatcacaggaggactgaggaggtggggggtgcccgagagggatgctttctcctacctgcctccctaagacatccctctgtttgtcctccaggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatgagcccttgctgggtgatcagcgccacacactgcttcatgtacggccctgggtttctcctcttcgactcttctgccccaccccaagcacatccctttctccttcccagcaaagtgttccgcctcatttctccctcatctgcccctgtccatgcgcccatggccttggggacaagtcgtgctttgaggcctctagggagggaaggaagaagtggcatgatttcatgggactaagctgtttgatgggtatcttcttccacagtgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattggtgagggggaacgcccgcgactactgtggccataatggcttggggagagtgggacccagggagagactggagctgagttgaagctgccggtggggcaggggtggggcgagggaccttgaagcctcgatatacatgacaaaggatggcagggaagagttccatgaagtctgaggggcctggtgctcctctggagagaccctgaatttccccaacaagtagccctcttgcgagtggaaacagccctgtgggtatatggcttgggctgggaaggccctgtttatatgaattagaaaaagacacaccttcctttgtgggatgcagcctctgtctgtgctaggatatagaacttggagaatggagccttgggatggattccagcctaactacctcagggggatcctctagagtgcagctgggagtttttgcagaaacgacctgtacagctgtatgcagtggctctggccatccaagcctttttcaacacctggaacaaagcccttggggcatggggcaggggaggtttccaggtgataagcgaccagcagacctccctggatgactgacctagggataggcatagctacttcctcggcacttggaggggacagatggggaccgcctaaccagtagtgatctttctcctctgaccctctgtcctcccccagccttgctgaagatccgttccaaggagggcaggtgtgcgcagccatcccggactatacagaccatctgcctgccctcgatgtataacgatccccagtttggcacaagctgtgagatcactggctttggaaaagagaattctagtaagtgacaattgcgactgacttagaaggtcctgaggagtgttttgacctgaaaatgagcccagtgtgatcaagggaagactgcagagttagaggtgggagcactgaggcggtggcagatgggtccagggatggatgaagagtgttgtttagggagcgatgggctgcaaaggtaaatagatggtaggggctataggtggaggtaaatggctcagatttgcatggagagagaataatgggcctctccctgggtgatgatactttatggtgtcccctctctggcgagacgtcccacgtggaggcagataaatcttgatgcaaacgcctccctgttttctccacctagccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccaggtgagtgttccaagcatctctctccacctcttccatatctccccagagctcctgggcttgttccagccagcttaagggtgtctctctctagccaaagccctaagtagccagaatcaggagctcaggtctttgagggtttaaaccagtccttatgtgtttgccagacattaccaaaaaaatcccagctctgcgctagtcacttcagactgggggcacgagatcctagaaagaggaaacagtaaaagacaatgtaactcagtgcccagggtgtgttgtgaactataaatgatcaggtgttcaggagagggaggtgagtgccaacctgagggtcagggaggggaggctttaaaggaaatgtgacttgataggcatttgaagaggcagagggaagaaaggaaggtgtttcagttgaaagatacaaaactgagaaggaggctggcatattccgggtggggaggagaactagggtctgggagtgtggatggaatagtggcagatgacagggcttttaaagccaagcaggggattttccaacttcgatgtggtagaaatggggctgcgtcaggcacagtggctcatgcctgtaatcccagcattgggctaggccgtagtcgatggatcattgaggccagagttgagaccggcctggaccaacatggtgaaaccctgtgtctactaaaaaatgcaaaaaaaaaaattagccaggtgtggtggtgcctgcctgtaatcccagctaatcaggaggctgagacatggaatcgcttgagcacaggaggcaagtttgacgtgagctgagatcacgtcattgcacgccagcctgggcgacagagcgagattctgtcctcccgccgaaaaaagaaagaaaatgggaagtcgctaaggactttgactgggaaactcttccctctctctggtatggttgggtgatgggatcagaaatcccctcctcacttctctagggctcatcttttgtatctttggcgtcacagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgtaagaagagactgggaagataggctctgcacagatggatttgcctgtgccacccaccagggtgaacgacaatagctttaccctcaggcataggcctgggtgctggctgcccagacccctctggccaggatggaggggtggtcctgactcaacatgttactgaccagcaacttgtctttttctggactgaagcctgcaggagttaaaaagggcagggcatctcctgtgcatgggtgaagggagagccagctcccccgacggtgggcatttgtgaggcccatggttgagaaatgaataatttcccaattaggaagtgtaacagctgaggtctcttgagggagcttagccaatgtgggagcagcggtttggggagcagagacactaacgacttcagggcagggctctgatattccatgaatgtatcaggaaatatatatgtgtgtgtatgtttgcacacttgtgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtacttattctgcagcatgacctgtgaccagcactgtctcagtttcactttcacatagatgtccctttcttggccagttatcccttccttttagcctagttcatccaatcctcactgggtggggtgaggaccactccttacactgaatatttatatttcactatttttatttatatttttgtaattttaaataaaagtgatcaataaaatgtgatttttctgatgacaaatctccctggtgcttgtatgggaaggagttggagtacataaaaaggagaaaataacaaaggtgg

SEQ ID NO: 56 - DNA, 852 bp, Homo Sapiens
cagctgcgctggaggctgaggccgattgcttgagcccaggatttggaggccagcatgcgcaacataatgagacccagtctctaaatgcatgcctctctatatattaaaattctgatgtgaaaatattttaaaatttaatacatttcaaatgtttttaattgtataataaacaaaatgtaaataataaaataatttaatattaaattcaaaaatgaggtagaaacaaagcacagcgatataaataataaattttcctttacatttttgaggcggtcttttgagttttggatttccttcttaggtcactgaaatgtgctccttggagccagcccgcaaatcacgcatttagaaaaacataactatacactcctaaccctaagtattagaagtgaaagtaatggaatctcgatgtaaacacaatatcacttttttgtagagctattttgagtataataaatttgaactgtgccaatgctgggagaaaaaatttaaaagaagaacggagcgaacagtagcttcctcgtccgctgactagaaacagtaggacgacactctcccgactggaggagagcgcttgcgctcgcactcagttggcgcccgccctcctgctttttctctagccgccctttcctctttctttcgcgctctagccacccgggaaggcactgcggtagctgggctctgattggctgctttgaaagtctacgggctacccgattggtgaatccggggccctttagcgcggtgagtttgaaactgctcgcacttggcttcaaagctggctcttggaaattgagcggagagcgacgcggttgttgtagctcgctgcggccgccgcggaataataagccg

SEQ ID NO: 57 - DNA, 2501 bp, Homo Sapiens
tcttgtcactccatgcactgtgttccgtatgctaaatagtttgagaaacccaaatgggccatgttcgcctacatttcattgtcctgtacttcctgtcctgtactagcaaagcagtcccattggtctttcttctcctcattaacaataaaggtaacacttttgatgttgtttcttcagaaaaccttcattcatcaaaactgcctcaaagatcatgtttgtttgattccagaacttcctgtaattacctgttattgtaacactcatcactgtattttacttacttgtgtaactaattttccatattctgcactagacaacaaagtcctttaagtcaggtactatatctatttacatagcattcacatctcctacaataagggacattagcagataaacaacacatattaaatgaataatgaagtttctgaaatactacagttgaaaactataggagctacattatatagaataaacatttactttgctatagaattcagtgtaacccaggcattattttatcctcaagtcttaggttggttggagaaagataacaaaaagaaacatgattgtgcagaaacagacaaacctttttggaaagcatttgaaaatggcattccccctccacagtgtgttcacagtgtgggcaaattcactgctctgtcgtactttctgaaaatgaagaactgttacaccaaggtgaattatttataaattatgtacttgcccagaagcgaacagacttttactatcataagaacccttccttggtgctctttatctacagaatccaagacctttcaagaaaggtcttggattcttttcttcaggacactaggacataaagccacctttttatgatttgttgaaatttctcactccatcccttttgctagtgatcatgggtcctcagaggtcagacttggtgtccttggataaagagcatgaagcaacagtggctgaaccagagttggaacccagatgctctttccactaagcatacaactttccattagataacacctccctcccaccccaaccaagcagctccagtgcaccactttctggagcataaacataccttaactttacaacttgagtggccttgaatactgttcctatctggaatgtgctgttctctttcatcttcctctattgaagccctcctattcctcaatgccttgctccaactgcctttggaagattctgctcttatgcctccactggaattaatgtcttagtaccacttgtctattctgctatatagtcagtccttacattgctttcttcttctgatagaccaaactctttaaggacaagtacctagtcttatctatttctagatcccccacattactcagaaagttactccataaatgtttgtggaactgatttctatgtgaagcacatgtgccccttcactctgttaacatgcattagaaaactaaatcttttgaaaagttgtagtatgccccctaagagcagtaacagttcctagaaactctctaaaatgcttagaaaaagatttattttaaattacctccccaataaaatgattggctggcttatcttcaccatcatgatagcatctgtaattaactgaaaaaaaataattatgccattaaaagaaaatcatccatgatcttgttctaacacctgccactctagtactatatctgtcacatggtactatgataaagttatctagaaataaaaaagcatacaattgataattcaccaaattgtggagcttcagtattttaaatgtatattaaaattaaattattttaaagatcaaagaaaactttcgtcatactccgtatttgataaggaacaaataggaagtgtgatgactcaggtttgccctgaggggatgggccatcagttgcaaatcgtggaatttcctctgacataatgaaaagatgagggtgcataagttctctagtagggtgatgatataaaaagccaccggagcactccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtaagcacatctttctgacctacagcgttttcctatgtctaaatgtgatccttagatagcaaagctattcttgatgctttggtaacaaacatcctttttattcagaaacagaatataatcttagcagtcaattaatgttaaattgaagatttagaaaaaactatatataacacttaggaaagtataaagtttgatcaatatagatattctgcttttataatttataccatgtagcatgcatatatttaacgtaaataagtaatttatagtatgtcctattgagaaccacggttacctatattatgtattaatattgagttgagcaaggtaactcagacaattccactccttgtagtatttcat

SEQ ID NO: 58 - DNA, 2501 bp, Homo Sapiens
attaattctgcaaattttaataaatgctttattttaagctaaatgctgagatgaaaaaatgaaaccatatgagttagcaaagtagaaaatataggcatattaatcagtaaatgcagaatgataaatgctccatcaatatgcacttgttgtagtgaggccaccgaggagggtgcaatcctctcaacctgggaggagcaggtaggacttcagatgtcatccaactcaaagatatagtgagggacttgatcaaacatttgccaagaccactatgagttaaatgaatagattaggcatttctccaatgttgcaagcttcgaatcatatccaaactcagaacaacatagcttggtcataatgatcccaaggatcctattggccattgtctttgagcctcaaaggaacatattaaaactccataatacccttttgatctattctgaagttaagtagtgaatttacatgatgatgacacaaacactgtaaaggacctctgggttacttgtttataagctagtatttcctgaatcaatttttctgatccctagatatttggtaggtgaagtcatacctatatatccccacaccctagaacagcatctccaacttatttttccctccttgtcttttagtgggagccacatcagtatccaagaggagatccagaagcctctccaaccaggtagggacagttatagattccagacctcagctatggcctttgttacagagtacaaatgttatatagtacaagtttattgtacacatcccattgagtctctgagctttagaattttcttgtagaatttaacagttttttcatgccgtatttacatattattgctagtatttagaattttcttctccaaatgtataacgtttattattgcattttttgtatccactaagtggaaaatcatgcattagatattgtagaagtagatacaacaatgaacaagaactggtcctgaccatgagaggaactgatgatccaatgggggagatagacctgcacgtgtttaataaaaggaagtggctattccggtttctttttgatgggcaagcattttgcaaggccttgggctatgtgtgtgcaaggctaagccagttagttaattgggatttttttaaaaaggcacttcactggggggaaaaggaacatagagttggttattgtccccttgcctataataaaaacctattatttttaattttttaactgggtttgcggttaaatctcacagcccaagagatttgccacttcagatggattccatacacttgcatttaagtatgcaaaaaaattccaattatccagcaatttaaccaaattattggtaacttttctaaaacaaaaaaaaattgtttcccttgttttggcagcaatttcagttacagtcctttactttctactcaagaaaatagtttcaaaaagttgatgtttgttgctaaaagaactatttttatgaataaatataaaactaagaagttatggtgtcccttttttaaaaaatgactcatcaaaagaaataactttttcctttctcttgtaagagaaaaaaattaatctcttttagaattgcaaacatatttccttgatggagaaaatcaattcacatggcatagtcgttatttatccagttcaaaaaccagagtagaatttactactctgtctccattttttctctccccacccccttaacccacattggattcagaaagcttcattctgcaatcagcattgtcctttatctttccagtaaagatagccttttggagtcgaagatgaggaaaagcctgtattttatagtcttggaagtgtcttcttttgccaggacagagagaggagcttcagcagtgagagcaactgaaggggttaatagtggaacttggctgggtgtctgttaaacttttttccctggctctgccctgggtttccccttgaagggatttccctccgcctctgcaacaagaccctttataaagcacagactttctatttcactccgcggtatctgcatcgggcctcactggcttcaggagctgaataccctcccaggcacacacaggtgggacacaaataagggttttggaaccactattttctcatcacgacagcaacttaaaatgcctgggaagatggtcgtgatccttggagcctcaaatatactttggataatgtttgcagcttgtaagttatttcccttcatctgtttcaaatgttagcattcaattttagccctggttttggcttcagtcagttttgcgatagtagtgaagtaaagacactaggattttaaacagtaggaaaagttaatttagtctaacttttaatatgcaattgagttttgctatataccattgtactgtcatagttagagctgaaaattgatgtttttggtatctttttttccaaaggcaattgagtaatttggattctgtctctagtcggtctgtctctttagtttcctatacttgacaatgaggtcaaacttagcaaataa

SEQ ID NO: 59 - DNA, 2501 bp, Homo Sapiens
ataaaaaaagacatgaaatgaatcggggaaaatatttgctacataactaagaatgaaggcccttaataaaatctgtaaaactatacacacttttaggaatgaatcaacaaataatttctatgaattagaaaaaagtgacaatccaactaaaaaatgaataagggatataagcaatgtgtttcacagaaaaaataaaaattgacaatgaagttatgaaaaaatgttcagtctccttagtaattgcacaaaacaaactaaaacaatgagacattacccctaagattagtaaatgttaaagaaaaataataattggtgagggtgtggggaagtgggcacttacacctatgtttggaaatataaattggtgcaaccttatagggagagcaatctcacaacattttccaaagacttacatgcacaaccctatggcagagaaatttattcctcttccaggattttttttccttcaaaaacagtgatgtggatgaaaaacacatgttcactactgcacagggtataacagctgaaaactggaaacgataatactcacattcccttcagtaggggaatggttaaataaattttacaagccatctggtagataccaggcatgagctaaaagttagggtccagttagagatggaaagcacaccagtaatttgaaagggaaaatgtaatatgaagaattattaactagtaaaagaaggctaactgctaaaggtacaagagcactcaagctgtctgcagtcagcaggccccggctggtgagcaggaagctgcccgctgggaggctgccaaagttccctgaaggtgagcaccactggttctacaagctgctggcagtcatggcgttaagagcaggaagagaagcaccagaacccggaagagaaatccagtcctctgctaggccttgcaccgtccctctggcgccctctactgacaaagccagtaaaattgtgccgctagcaaaggagatctttttatgggatgtagcttggtgtcaccaaagagaacagagtggacttggagctcagatgcaacacaatgattgatactggcacagtatacttaccctgcttttgtaaacaaaatggtatatgtgatgtctctctttgtctctctgtatataaaacaatatttgtttctacttattatgtatttatgtctttactctgcatgccaggagctaagtattttgcatgtattaactcattttgttctcataataaccttcacatgcaggaatcattatagctactttatgaatgagccgaggaaggcactgagacgttaagtaacttgcccaaggtcacgcagctagtaagtggcagagcaagaattactatggctttataagcctaggaaaaagtctgaaagaatcaaaatgttaacagcggggacctcaaggaagcattgaagaggccatgggagaagttttcactttgttaaaaaatcagtccttcaaataaataaatacagtgaggcttccccagaagcagatgtcactatgcttcctgtacagcctgtggaactgtgagccagttaaacctcttttctttataaattatccagtcttaggtatttctttataacagtgctaggatgagctgatacagtttcctacactgtaacctaaggcaatgctttgcacaaagggatgagccagattgcttagtaattaaaacgcaaatacaaaccacaagcatatccattcatgaattggggggctgctttgtgtgcatagataaggtatattttttaaaaaaattatttttccaagaagaaaataaaccagttaataaacgacaactcacagtgccaggaagtgagaaacaagtgtgtgataaacggtggagaatgggagcactctccgcagtgggcgggaggagacgaggagggcgttccctggggagtggcagtggttggagcaaaggtttggaggaggtaagtcatgtgctctgagtttttggtttctgtttcaccttgtgtctgagctggtctgaaggctggttgttcagactgagcttcctgcctgcctgtaccccgccaacagcttcagaagaaggtgactggtggctgcctgaggaataccagtgggcaagagaattagcatttctggagcatctgctgtctgtgagattaagcactatgtatattgctttattcactccccacagcaaccttaccaagcagttcttttccacgtgaaaagatggaggctgggtggagcaaaaggaggtatttagagtcctcagcaagtgagaggcagagctgggatttgaatccagatctgcctgatactgaagtctaggctggttccacctctccggactgctttccagggagtagaagacagatattttaccttagctggctgcttctagaagtctgaccctgctggctcaaaacgactttagttccttgcccagaggctgcgggctgcgggtcaagacatcagtagaaggagggcccagccagagaggctgacatgggcttctact

SEQ ID NO: 60 - DNA, 2501 bp, Homo Sapiens
cgggcaggaataatcactgcctcccatccccttaaacatgccaagatgctttatccctaggatgaggtgacttactccaggtaactcctattgcctaaccactgaccaattactctgccctttagtctttatgtcattaaatctgcattaagaatttcatggaataggcccggcatggtggctcatgcctgtaatcccagcaccttgggagaccgaggtgggaggatcacttgaggtcagcagttcgagaccagcctggacaacatggcgaaaccccatctctactaaaaacacaaaataactagccaggtgtggtggtgggcacctgtaatcccagctatttgggaagctgaggcagcaggagaatcgcttgaactggggaggcagaggttgcagtgagtcgagatcgtgccagtgcactccagcctgggcgacagagcgagactctgtctcaaaaaaaaaaaaaaaaaaaactcagggaatggatagcagcattgatgaatattgcgtctggagagatcagatcacttgtcacttgtttccaggcacagggcttaccaagaggcagattccagatttaaataattctgtaacagcaaagtccaagctattttcactgctttggagaaaagacccagacccagagcttgaacctcactttgcagcaccccagttctaatcttttaagtttttttttttttttttttttttttctgctgggcacggtggttcatgcctataatcccagcactttgggaagccgagggggaaggatcgcttgaggccaggagttcgaaaccagtctgggcaacatggcaaaaccccatctctacaaaaaatacaaaaattaggccagagtggtggcgcgcacctgtagttccagctacgtgagaggcggaggtgggagaatcgcttgaacccgggaggcagaggttgcaatgagctcagatcccgccactgcactccaggttgggcgacagagcgataccctgtgtgaaacttttttttttttctccaacgggctttccagagaagtgtgtgtatgtgcgtgtgtgtgcgcgagcgtgcttgcttgggcttaaactttctgtcgggccacactttcccaagtctttgcactggctgtagggtgggctttatcctcgggacgtcctcctccccaagtccagcctgcagctggaagtcttcactgatctccatctctcctccctgatctccgtctctcctccctgcccgcctcaggactgggaggccgatctctctctctcgccctcccctccaccagccttttccagatgtatgtctgccaaagaccccccagtgcagaggatgatgaatgaagatcctcgagccagcccggtgggaaagtttcgtcgcctacaaaagcgagggaaagggaagggaagttgggggtaggggaaaagttagagctgagaggctggggcgcgacgagtctggacaccgggcggggacccaagctctctccgctcagccaataactgtgcctcccttaggaaggcgtgaggaaatgctccaatcaatccctgcactcctcccttggaatttgggctgtatttttttatttactgcaaaccccacaatccacccaggggtttccccagtgtttgcctccagcggtcccggtgcccatttactagtgctgctccctctcttccgcaagactgcgctccagtcccagcctccttctccgcgggtgcctcccaaaccgttctatcattctcgggttcagggaggcggaatcgtgcctgctctccggttcctttaagaggcgtcggctccacccctctcagagtcgcggtctgacgcgagatgacagcaacgagttcggtatgtctatgcaaataagcgccctcttgtgggccaatggggagcggaggtgccggaaccacggaccaatggggcgggggcgctggggctcaccatataaggagcggcctcgccataaaaggaaacattgtatctctttatatggggggaagggtcgggggatccctccgccgccagcgcgtggtcccggccccctccacccgccgtctcggccgcggccagcagcccctgccccccgggggacgctgacggccgcccggcgcgccgccctagcagacggacagggggcgctgcgcgcggcctggggcaacccgggccacaggggcaggaaagtgagggcccaggtcggcccgggcgtgcaggggccccgggttcgcagcggcggccgcggcagcgatagcggcactagcagcagcgggagtgccgggttgagccgggaagccgatggcggcggctgcggcggctccgattcctcgctgactgcccgtccgccctcctgcatcgagcgccatgttaccgacccaagctggggccgcggcggctctgggccggggctcggccctggggggcagcctgaaccggaccccgacggggcggccgggcggcggcggcgggacacgcggggctaacgggggccgggtccccgggaatgg

SEQ ID NO: 61 - DNA, 2501 bp, Homo Sapiens
ggaaccctctgatagagagggctgactgtatttattgaaaacaaaacaaaacaaaacaagggttgtattggtggacccatgcagctcaaacccttgttgttcccaggtcaactgtatatccagagcttataggaaaatacctctcccagtaaccctgctcaccatttctctcttaagctattattatgattagccacggtttgctatttaaatttaaatttaaataaaaatgtggcctttcagttatgctagccacatttaaagtgctcaatagccatatgtggctaatggttactatttcggacagcacatatttagaacattcccatcatttcagaaattttcattgggaacactctgcggaaaaagggggccatcataatgtgagtccatcttctggaaaaatcctgggaaggggacaaaggaggtctgtttggcattgtgtaatggtaatttggtatttaattttcaaaaatgtttacccaattcctattcatcagccaggtgtggtggctcttgcctgtaatcccagcactctgggaggccgaggtgggaggactgctgcagcccaggagtttgagaccagcctgggtaataatagggagatcctgtttctacaaaacaccaaaaacaaaacaacaactttgatgttgtggagtcaggacagtcctgggttaaaacctttgctctccttagctgtgtaaaccgtgggtctcagctttcttatctgttaacggtaggtacttcttcctagggctgttttgaggattaagtgaaagtccaagattgtgtctggcacacagtagcttctcagcaaatgttttcctcctatgtcagggaatggctcctttatcccgttttgggcccatgggtggccctgaagggtgggtgctcaggtgttaagttctgtagatggcatatccttgggaaaagcaaggcaattaaaaacagtgagaggttgctctggttaagttttctcctataactttccccatgggtcaattgggtagaatctgccattttcctaatacttactgatggtagtggcattcggaagcacaatagctgaagccggagctctgagtggagagaaaggtctgtttctcaggcccaaaaagaggttacacacccatggctgtccagtttggtggtgcaggccctgaaatcagaccaaactggatttaaatccccaaacctatactctaagctatgtgaccttgggctagatacttcacctctctggccttatgaagtaggaataataataataccgtctaggttgttaggagtattaaatgaggtaaagcactgaaaacgtttagggactgtgttaaatcattaaataaataaaaacggggatgaccttatcggcttgacacaggggattaaatgagataatatatgaagacaagtacacggcaaatgcttaattaatgttgcttatttttatgtctgcaaactgacttaaaggggaggcctttaagaaagacagtggggcaatttgcgcgttgatgcattgtaggagaaaatgtgcagggggcccgttgggaccagagttcaaccaggtaagcggcagaaaaccacaaatacctccaggcgttcctggggcagcgccgcctccccaaaatcacgcaaaacttggtttgctaagaattgtcagctcttctaaaggaggcgcttcacgcatctcagtctgtgaaatgggacccaggacccaggtagaggtgcgttctcggcctggggaccgagtattttgtgcgctccggtaacgcaggaagacagcgccactgacactctagagaccagcgggcaccgcctggaggcgccttcaccacttggcggttccgggtccgcgccccaccgcgccacaagactcacgcccgaaccacgtgatcagggccgtggctccgccccgctcccgcgccgcgcgccgcttccggtaggggcggaaagcggaagtgtgggagggtctgcggggcgggctcaggaggtccgcgggaggatggagcagtgagcgggtctgggcggctgctggcagcgccatggagacggtacagctgaggaacccgccgcgccggtgaggggccactggctaagaggacgggcatggggtcaggggaagaaaaggcgggaactggttgaggggatacacctgtgtgggagtccccggagctaagcgacccagccgatggggcacctgctgagtgaggggggggacgtctggtgggtgagggtccggctgaggggagcatctgctaaggaggttagacttgggaccggttagagggagcactcgctgtggtgagactgtgctgaggaacgtggggacaagttagggagagtacctgctgaggccgggccactcgggggaacgctatccaagcagggactcacggaggtgggggcgaatgctgaagcagggtgagaatctgtgagggatctctttaagggggtggatcgagaactggccaagaggaaggccgggtggactttctaagggt

SEQ ID NO: 62 - DNA, 2501 bp, Homo Sapiens
gcatggtggctcacgcctgtaatcccagcattttgggaggccaaggcaggcagatcacgaggtcaggagatcgagaccatcctggcgaacacggtgaaaccccgtctctactaaaaatacaaaaaattagccgggcatggtggcgggcgcctatagtcccagctactcgggaggctgaggcaggagaatggcgtgagcccaggaggcagagcttgcggtgagctgagatgatcgggccactgtactccagcctgggcaacagagtgaggctccgtctcaaaaaaaaaaaaaattactacatgatactaagtaatgcggaaggtgactcaaagggggaaaggaacacagcagtgtaaaggaaggaggttgtagatggatctagaatttccccctcatttccatcaggtgaaagcctgagaaaactgcaatctttgtgcaggctgggtttgctttgtacacactggtcccctagtgttcatctccaataatgctgacaactctgaaaaccatctgtagacattctgcaggctccatctcaggaacaatggctattttttcgggtagttgaagcaaaattaagtccaatgataagcaaatataaccattatcaaaatcttccatttatgtttgttaaagcaacctaagtatgatctgagaaggactctgtattctatatttgagtccttgtggatgaactgtaacctagcttaataggcagacaagattgaaaacctaatttaggagtatgtgcctttaacaatagctgagtcttggccaatcccagtggccatacttcaaccattcatacactgctgagtgttcaaactgtgttcaaagaaggcaaaagccaacctgtaaccaatccagttgtttctctgccttacctccaatttctgtatgtcacttccctttttttgtctataaatatgttctgaccatgaggcatccctggagtctctgaatccgctgtgattctggaagctgccccattcgcaaatcattcattactcaattaaactgctttaaatttaattctgctgaagttttcttttaacaggtttagaaaaaataatggcaaaaatgaatgaaaatccaataaccctggaagcagaaaaggctgggggctccaataagtgtaaatagtcccatccctatattttctccatggcaattacaatccagcacattatatatatatttttttgcttctcgcattttggcttagggtaaagctttttaaaacaggcactgccaaccagtgttatcaagaaggtctggatgccgttttgtgggaacattttaaagaggaatgtccaaaaggaaaagggggatgggttgggagaagggtatcaggcgggtatctcaaaaccattcttagggctataggtttaatttatttggttgtggacgtcagagccgtcatggtaagaaggaagcaaagccttttgtaataattaaagccttcagaagcagcgtgccccattgcccactagtgcgccgtgaagtctggtgttcacctacagggtccctctcagcactgcccaggcctcccgagtgctccagcacagtagcttggagcttgttggtttggtgaccaagatacactccagggaatatgccatgcagtggagtctcttccccggcactgcatagcaaaaggaaagggccgctgggtgtctgtgggtcctgggcagtcacagaagccaccgcgctggcggggaggagggggaccgatgcggtccatgtcccgggcagccccactttctctgcctgcgaagggcccttgtccggcgggaggagagaggcgcgccccacccgggctcctctacacctgccgccgcctgggccgattccgcgggcctcgcccggcgcttcagccgattcccgcccagctccgggctcatgggcgcggtcagcagggcgggccagggcggcggggcgcgacactgggaggaagtgcgggccgcctgcccgggcgcgttaaggaagttgcccaaaatgaggaagagccgcgggcccggcggctgaggccaccccggcggcggctggagagcgaggaggagcgggtggccccgcgctgcgcccgccctcgcctcacctggcgcaggtaggtgtggccgcgtcccctacccggccgggactttctggtaaggagaggaggttacggggaacgacgcgctgctttcatgccctttcttgttctaccttcatcggccgaggtaaaagtgctgaaaccatgtgaataaaatacaggtgggttccgccagcttcgctcctgaacctacccgcgctcgggatccagaagctgcgccgggagagaggggctcaggcctgggcggaggggacggaggtcagaccgtgcggaaagtgacccgggcaccccagggcgcccaggcccccagggagcgcggaaagtgcggtcgcggcccggccctcgggagacgcgggattgggatcaggcacagcgcgaggaagtcgatcttggagctagaacattttcc

SEQ ID NO: 63 - DNA, 2501 bp, Homo Sapiens
cccaaaagatacaaaggggtataaggtgaaaaattattctaacccatccctcagtgacctagttcccttcctctgaggtgaccaatttcttgtgtatctttcctgagataatctatacatatagcaccatatacaagcaaatgaaatatgttttatttatttttttgagactgggtctcactctatcacccaggctggagtgcagtgacaccatcttggctctccgcaacctctgcctcctgggctcaggtgatcctcccaccttaacctccagagtagctgggactacacgctcacaccaccacacccacctaatttttgtttttttgtagagacggggtttcaccatgttgcccaggctggtctcaaactcctgagttcaagtgatctgcccacctcggcctcccaaagtgctgagattacaggcgtgagcctccacgcccggccccaaaatctgttttaaaagcagacatttcttggtgattctaataaagggggttctcagacatatttggaaaaatatatccctacttttatgccagaccctgtgctgggtccccgggctgtgtgacctgacactgcacagtcctgcttagaatgcttaaagagagttaataaggtaccaccttctatgccataggcggggagcaaaggggctccagtgggccctgcctaggaggcctgaagctagagctgctgagggcagggctgtgctgcaaagaaaatgtctgagagctgcaggcgtttcatcttctgtcatcagctgtggcacctggcagacactggataggcttgtagacaaagacctggtaactcaaggagctgcttggccttcctgcccagtcccatcccagaggcactgtacatctctggtttcttcagggggccctgtgtggaagtatcttttgtcttcctggtgtcagggatatcatcacgtgcctgttggctaggcgagcccggcgcccagtctcctaggatggggagagtaatgttcccgagcagaacagggtggggctttcagactactccctttcctttacagctggcttcattccatcgacctcatcaaagccttcctgggagcaccctagagaagagttacgtccaggccgggccctggctgcctggttcacggcggaatccccagcaccacgcctcgcacgtcgggctcaaagcatgtttagtgaaggagtaggtacctactgctagatggagccatctctctagacttggggtttccctataacgatggctatgtttggcatggaagcctctttagaagtcaatagtaggaaataagggctaacagcacctaattgtggagtaaggttcaaatcctagctctgccacttaaccgttccgaacctgttccctcactgcagaggcgaaaaggctaacactatttcacctcggagggttaccgtggagaatggaagctggacaagctgtatcagttcagtagtaaaacacacacacacaagcgccccacccccaccccaccccaccccaggaatgaacacacacacccgcgcgcgcacatacacctcaggaatgaacacacgcgcgtacacacacacgcagccccccccaggagtgaacacacacacacacgccccgttctgttgttcccaggaacacacacagagacgcacacactcgcccggttttgttttttccaggctttttaactggggtctttcactcggcttagggcaccgctgcctgaaagacctttctaggccagtcggggtccggcacccagttgacgagacagcgcggcgctttcagagctggggagaggcgaaaactcttccggccccccgatcccccggccagccgcccccggcagctccttgccgcctcccggcctgggcccgcccagccgttctcggcctgccgtcaggcgatctcggcggccagcccagccgcgatgtgacgccgcgcgccccggggtcctcggcgcctgcgccctctcctataaagcagacgccgcgccgcgctgcgacgctgtagtggcttcgtcttcggtttttctcttccttcgctaacgcctcccggctctcgtcagcctcccgccggccgtctccttaacaccgaacaccgtgagtagccgcccactgaactggaaagggtcgtggctaccggattgcgtgccggctggcctcaccgctgcggtttgggcctgcccgcggcgggcggtgactgggcctggccttctttcgggcccggtggatcgcgttgtcgaccctgttcttcgggagacactaccaggttccgttcacctgccccgcccccgactcagcgaggcctcctctggccgggcgtcctcacggcgctccataagtgagccgaaccccgggctgggccttctctgcaccggccgagcgtcagccggcgcggagctcggctgcaaggcccaggctgcggccgggggcctctcttggtcttaagcctgctgtcccggggaccagggcgggggtggcggcggggttgtgaatggg

SEQ ID NO: 64 - DNA, 2501 bp, Homo Sapiens
gatctgacaggttaaaggtgtacacttattttctctgtaagaagcgtcatctggtaagatgatcaagaatggtgcaaagcaggatggggagtttaaaattgtttccaaatgtgggaatgtaaatgaatataaacatgtaagattttaatataccaaactgatcagattctgtgtaatttccaagtttcttttttctttcaaaactcctctgaaatctgactgtccacaaaaacttactttatagaattttatgtgatttatttactcagatattatactgacctcacatccagtagtgaaaacagattttattgtagaatctggaaagatagagggccatataggttgtattttcagttttgtttatactaacacgtgtttacaacccagtttaatttacaccctgtattgtattattgttgtcatatctctgtatgcatgtaagtataatatgtgttggcaaaggaaaattttgagtaagaagaagctctctgatctatttgattcaatatgtatttgagtgtctaacagacactgttttagacactggtgatacaacactgaacggagcaccaaatactttacagcgtctcctggagctgttgtcaagacatactttccaaggggaatatttcagaataggtgataactagtcaacgaaggaaaagtaccttagtcatctaggagagttgtacttagagtgaactgaaataaactaagctcacgaaagacagagattttttgtttggcttttgtctgttgcattcactactgtatctccagggcccaaaatagtgctcggctcataataagtattcagcaaatatatgttgttgattggagtgtttgttttgaatttctgtaatcaaacacataccttggtaaattatctttacatcttgctagttgaaaattttatctcagttgctttgtttttaatgttaccttgctttttgtttctacttgtgccatacatcaggatgctggaaaagcttattaatattgacagtcatatggttatctgatattgaaaagaatagatttggaaaggaacctaagaggtcatcttttgttcagcttcctgcctaggaaaactaagtaagatgattaggtatgtatatttaattagtcatttaaaaaaaaaccaggacaacataattgagttccctcttgagaaaatggagaaaggtacttaaccctagctataaagggactaacctggaaattttagaacttctgtgtgggaaagtggaaaaaaaaaaaaagcacaactaagctgctctttgttgatatcagaaatgggcctgtcattcattttggcattgaagcatagcctcctatctcggggcaggactgggacatttttttcctcccacaagagctggacagttattacaggttcaaaaagccccgaccagtttttcaagagtttctcctcctcttttccccctgaaactcgtggtgcttttgctctgctttcaagatgcattaagtctcctgctttgtgactgctttggagccagcagatactctgatatgtataattcaaattatgcaggtttcacgagtaagtttaatcttattttttaagttagttaaaaggcaagtgatatttagaaaaatgttaacttgtagttatttcaccctttttactttaagcatttttattgcttctcggccttttggctaagatcaagtgtgtactttaagcattttttaaaataaaaatatccttttaatttaataagaaaacaaggttctacatagaaaagccccttcatctaagacctgcacttttcaatttcttttgagatgtctttgttgtaaacagtattcatatgtcttttgaaagccagttaactaaacagttttcttgagcatctttttagttttactgagaagtattttaaattgagcttttctgagctcgattgcttacgtctgacacagtctcaagtttccactgaatggtaacaaagactgtagaatgttgttggtactgcagtgagaggcatgcttccttagaccaggtaagagagatcagtttgtttctcactgctgggtgagtttttacagctcttattttatattctttaagcagcagcaatattaaattgataaatagccaggagcacgctgatttcaagacgtccttgcttgttgcagacagaaaaactacagggttatgtatgggggttggggtggggggggaggggaagaattagtttattactcagttacttatataaattaattaaaatgtgaaaataattctggagctcagttttcttaattcaggaactaaagcagcagttgaggaaatcagtaattttaaaggtacttcatggttattacttgtgaaagcaattcaaaggatagtttttactttcatttttttccccagtagttaataaaataagctttgcccttaactaaacattttttccacttacgaaaacttttaaattgccaacagcaaaatatacttcccaaggatcctt

SEQ ID NO: 65 - DNA, 2501 bp, Homo Sapiens
cacaagtcaagaccgctccctgcttcttagcccgctggggagccaggccagcaggccccacattcctgaggaagggacagggttctggcctggagggtctagcagaagccaccccaggggagggcccgacaggaaggaaggtaggcctgccggaggggcatacaggagcttcctctcccgccacagtgtccagggccaactgctccagccctcaggctgggtcaacaggatgggacagcccaggcggaaggaaacctgtggggagggacaccccgcagacagaagcagggacatggggtggggagaggcaggaagagctgccgggctgctgagctggcgcctctccagcagactcaggaggggcggtgacaggaggccattccctcctcatccccgcagccctgggcctctctggtcctggccaacagtattactatcattattattgctgttgttcgctagcctgggccttagatacattagaaaaaaaccatcggaagatacgcatagcattggcagtttctaaaagaattaattcccttcctgtgttcattctgtgattactgggatagaaatgctatttgcattaccagcctttcattcagttacagagacgtgagtgctcgaaggagagacagtgatttttgccttaaattcagcctgtccaaatcggataagatctccgatttgctttaagccccgttatcactgccttcctctccaacaacagctgctgtgatcacgcacaaacggccaaacgggggcaaatccgtgccaaagcagggccatgggctttcctgatcagaaggcctagccccagcccccaggcgcagcacacgggcggcttcctttcagaaacccagcctgcctcccaccagctggagtgggtgggtggggcggtagtggtgccagtttcagggaacggccggcaaacccacctccaggcgtgctccagcgggagcctggagaccctaggagagccctccccacaagcggcttccaggcaggacgcttccagaggtcttggtccaggggtgggggtgaggtggggtctacctttgaaacagctacaatttaaacttcagctacaccgagctcaaactcgattccgcagccgagtgtcggcgccagagaaggataaaaactcgggtctacggctccccaccacgcccctggtccggtcctctgggcttccaggagtcctcacgccatcctctgggttgcccaggaggaaggatgggcggggcgggcaggcgctgcgggcgctgcagatggggagggcgagcccgcggcacggcgtgagcgggggagaggcgcgcgagcaggtgtcggctccgtgacagggtcccccatcccgcgccccagtgctccccgaggcttagtgaggcaaaacccagcaaatgcttcagaaatgcagctcagtcggtcaccgggttctgcttcctcatcagacgcgcaagaggatggcgcttccaatgcaaatctcttggctccggccccttggctggcagccgccgcgtcccccgcctgcctggcgtcccgcccactccgtggcgggctgagacgaggcccggcgcggaggggacggggcggagcgggcatccctccccaccccccacgtggggctggccctccgcagtgcctgggcgcgctgcagtcgccgcgcctccccggccgcggcaccgcctctctaggcaggggcgggggacgaggggcaaggagtgggcgaggggtgggcgaggggcggggggcgtcactcaatcaggtggcctctggagttccccggggcagggcagagggaacacgctgccggggattgtgtacacgctccactgacaccagcttcacgctgccgggcagtcgccgatcacgcgtggccccgcgagcccattggccggcgcctcacacacctttgccgttgattggccggcctcaggctccgcccccacccccgcccgcggcgcggggcaggctgagcggctacctgaatggggagggggcagacggcgctgagcgcggcggcggcgggagcggcgtcgagtgtctccgtgcgcccgtctgtggccaagcagccagcagcctagcagccagtcagcttgccgccggcggccaagcagccaaccatgctcaacttcggtgcctctctccagcagactgcggtaagtcatttggggatgcccctgtgcttcctcgcctggtcttgtctggggggccaaagggggcgcgaaccccgagccccggacatcagccatgcctgagaattggggctgcagcggagtcgtggggaaggaaagggcttcctgcctgcagactatgggcattagtgagggcgtgtgtgttggggagggggtcgaaccagggggctgggatcttcagacagggacaggggtcttgctctagatgtactgaggggaagggacaactccgcatggagacccgagagggctggtgaggaggaggatgacgagcgggggaggagtggggagggggccgttgccctg

SEQ ID NO: 66 - DNA, 2501 bp, Homo Sapiens
ggggctgtagaaatggcggccccatctcccaacaacttgggcattgtgaatatcacctccttaaaggggatctcctttggtcatcccgtctagagcagccaccataacttctgagcgtttattgctagctgatatatatcagaaaaatacaaattccacaaaagcagggactggtctgcttctctccctgcagggcccaggttctggcacatagttggtgcagaaagtgtgcagcctcaggtcctatccaagcccccagggcatcacactcgggacttgttctgcatatttttacttttgcctcccactggtactagttcttccgtggaacagcctgagtcccttcagatacttaatgttttttctcaagtgctgccatgaagccagatctccaccgtcttggggcattcctttttagggatgggaagtatatgtcgctccttttatgtgatttacattctatcttggataatttggccatcaccgtagttcattcagatctgtttggatcctgcccatctcagcttcagtccatttcattcttttaaatctgatcgacagttacctccaacagcttcatcacaaatcactcacaaaaatggccttaatcctgaagtttatttacggagagcacacttgctaggtgtgtggcagatatacaggaagcacaagatgaggcagcagatctagaggcaaatgacttccttctccctgcctagtggtgactgccagcatcacgccctcccgggagaggtgagaaacccctccacgcaagcactggaaccttcacagtcaagagtggcaacagctccggttactggacttgggcctgttgaattctaatactctgtgactccacatctgggctgaatttttgctgagtatgatggaatttacatgcttcctccctagcccctacttgtctgtatagttggaatatttggttgcctcctctggagggatctagtacgtttagagtctagacgctggaactgtcaaagttcagaggaaagagctccagctgcaaagcaagagaaatgggctggaattctagcttcaccccttaatgaatgcttctgattttttttttttttttttttttgagacgtagtctcactctatcgcccaggctggattgcagtggccacgatctcagctcactgcaacctccgcctcccagactcaagcgattctcgtgcctgagcctcctgagtagctgggattacaggcgtgcgctaccacgcccggctaatttttgtatttttagtagagacagtttttggccatgttggtcaggctggtcttgaactcatgacctcaagtgatctaccttcctcggcctccgaaagtgctgggattacaggcccgagccaccgcgcccagccgcttctgatcattaaaaaaaaattttttttttggcggggggaacgaagtgtccctctgttgctcaggctggagtgcagtgcagtgatctcggctcactgcaatctctgcctcccaggttcaagcgattttcctgcctcagcctcctgagtagctgggaatacgggtgccccccaccacacccagctaatttttgcatttttagtagcgatggggtttcgccatgttggccaaggctggtctcgaacttctggcctcaggtgatctgccttccttggcctcccaaagtgctgggattacaggcgtgagccaccgtgcctggccaaaaaatttatgttttaaaaagactagtcaagtgcagtagtgagaaggggggaaagagtagagcaaggagttatatctgttgcttctgaccattttgaacaagttacctaattctctgaggacaagctcggagaatgggagagacagttatctatttgcagggttgttgggaggaataagtgacatcatgagtgtgtgccaggtgtctgattacagaaggtgttcaattaatctgcaatcattaattaacccttcagtcgctggtattatttgccatccatcctccgagtgttgccaagttatgggtgcgttctgccagcgtcctagcagtggtaaggcttctggctgccagcggcgaacctctcccttcgagtatttctcctcttgctgagatgaaatgcgaccgggtctctttaagggccaggcgccgggatccaggcggcgcccaacggctggactagcagtcgtccgcgccgactcgcacaagaaggaaccccgggcctctggatccgctcgcccggctatgctgctgtggccgctgcggggctgggccgcccgggcgctgcgctgctttgggccgggaagtcgcgggagcccggcctcaggccccgggccgcggagggtgcagcgccgggcctggcctcccggtaacgcgcgtcttggtcccgcctcccaggagcccctatgcgcccacctactcccggcccctcggcttccggaacccgcccgagcccgaagcgcctcttccgaggcgcgggatttcctccccggctgcggctgggacgggggcggcc

SEQ ID NO: 67 - DNA, 2501 bp, Homo Sapiens
atggtctcgatttcctgacctcatgatccgcccacctcggcctcccaaagtgctgggattacaggcgtgagccactgtgcccggcctctatcagcattttctttctttttctttttctttttttttttttgagacagagtttagctcttgttgcccaggctgaagggcaatggtgtgatctcggctcactgcaacttctgcctcccaagttcaagcgattctcctgcctcagcctcctgaatagctgggattacaggtgcccaccaccatgcccagctaatttttgcatttttagtagagacagggtttcaccatgttggccagtctggtcttgaactcctgacctcaggtgatccgcccgcctccacctcccaaagtgctgggattacaggtgtgaaagagaccattcccgatctctttcagcattttcatactgaatgtccacagctgccctgtgaggaggctttttacccatattttctgactcagagagaagcagccacatgtcccttggccatggcagttaagaccaactccatggagctgggtgtcttagctcacatctgtaatcccagcactttggaaagccaaggcaggatgattgcttgaggccagaagttcaagaccagcctgggcaacatagccagaccccatctctacaaaaatttaaaaattagccacaaaatttaaaaattaacaacaaaagggccgggtgcggtggctcacgcctgtaatcccagcgctttgggagggtggatcacgaggtcaggagttcgagaccagcctggccaagatggtgaaatcccatctctactaaaaatacaaaaattagccgggcgtggtggcgggcgcctgttgtcccagctacccaggaggctgaggcaggagaatcgcttgaatccgggagtctgaggttgcagtgagccgagatcgcagcattgcactccagcctgggcgacaagagcgaaactccatcttaaaaaaaaaaaaaaaaaaaagtggaagatgaggaagttgatcagacatcaaggatgagcggatgacttaataggcttctttgctaagacttggctgggcaggtgaaagacaaagtcgaggagtggttatggtgtggcacagaagaagggtcagaggacggtctttgttacctcttcatgcctgagtttcttcctctgtgaaatggggataataagagccgccatacagggaattgctgctaggatcaaatgagataatgtatgtgaaacgctctggctgtaggcttctcagcaaatgggcacgacttgcggagtggggatttgaattcacgtctggcgggatgtccaagctgctaccctgaccgctagggagcttcagaggacagggctgcaggtgatcaggaagaggactggggcaggtgggcgaggaatgcctcccaggagtgaaggagggggaattctagtcagcaggatggagtcggccaggtagaaacgagggaaaggagacaggaccggatggaacggggaagccaaagggcagggcgtcggagggttgaatggtggccggtgcagctttgaacaccgaggtgaggacatgcagctgtgtcctagggtcaggaccgtacacgcctgacccaattccacagcacggaggggaactccaggatccggccgcgttgcccacacacttcgctctccctcccgcctctcgcaagcccctcccccgtctccgtccaccgagtgccagccaatagcagaagcgacagcgcatctgggtgccgactcagccaatcgcggctgagtgacgaatgagccccaggaccaatgagagtgccgccaccatggcaaaaaaaaaaaaatccaatggtgacgagcagggagaacagagcagctgccaatgggcgtgtgcgtttcaggcggccaatgggaggaggcgtctcggcgggggacaagcagtagctacccgcgggagcggggaggggtccgggttcgagcttgtgttcccccggaagggtgagtctggacgcgggcgcggaaggagcgcggccggaggtcctcaggaagaagccgcggggactggctgcgcttgacaggctgcacttggatgggagcacctggtgcctcgggactgctccgatgcccggtgggtgcacatcccagttcccgccgttgccggccgggtttagaggttttggggggaggacatgggggcgtgcagccttcccagttgcaaacttcactccgaccctgtcttcaaagctgggtctgggtccagtggggacgagaaaggaggaaggaggaagtaggctccgcgaaagccccatccccgggatctcatctataacatgaataggtattaatggcaaaggctaattaagcgcttactgtataccaggcactttctctgcctcctcgcgttaaatcctcccagcagccttttgaggtagacactgttacatgcccattttccagatgaggaaaccagcaacatgggtggaagtgacagcccctccacttccatac

SEQ ID NO: 68 - DNA, 2455 bp, Homo Sapiens
ggagtgcaagaacacagaactaaaacagagcttgaaacttaaagaaagggagagacttgggggaggagtggggtggagtgacgtgatgtgctgctggaaaccagcagttggtggtttcctcttgtgcttcctcttctgtgggttttctcctgcttgtgggagggcctttttctctcctcccgacagaaaggctatctttggtgttcgttcccttgaactgtaacatcctgtaagggtatgattccatgcctctgtgtgggtgtgaattccctcatggtgaccctcaaaatctgcacacaggaccccttcccattgaggggaggggatcaaaacaactctacttctcagggtcctctcctgttccaactggtctgtgtccaagagaagccttaggtaaatggggccagcttgaagatcaaacaggtttggcagcctctcccggcctctcttttctctcctacagctttatagctacagctgccttgatatcaatattgactttggctggctggcatgactacccacagggtatcgtgccttaatttaccaggtgacaggcaacgctgccctctcctggaaccatccagcagagccagggctgtacccccaaatcctgcaacagaggtttccctccatctcacctccctgtccctgcatttctcctatctcagtagctcctctttccctctctgggcttctctttccactccctccccttcctgggcttggtaaactagtccctaatctcttcacaccccagattggaaggtgggtccctccctgacactccccagagctgtcaccaacctcctccaagtttctatagctccattgctcaacagatttgccaggggtaaccattaacccagcccttaactctgttcccccacctttcttgctggaggggattttccaattactggttagcacagctaggtcatctcacccccaccatctttcctaacttcttgggttggggggctggggaggaatctccccatctcagggtactaggaacaaagctggggaggatggtgcatttaaagggattatatatatatatatatatattttttttctttctccctcataaccccacccccgcaacacacacacacacacacacacacacacacacacacacacacacagacgcacaaataagctttatggagcagtgacttcattatgttcaccgctttgagtccaacccctggcccaaaataggcactaaatagttgccgaatgcatgaatgatagatacctctctgtcttcaggggtgtgtagaagtgcgaaggggtatgggcatgtcccagtaggggtgtgagtgttctgatcagaactacttctctctgccagaatttgatgtaattcgaatgcttccacctctgcttgaagggtttaaataataaattaggccctgtcgtgccattatgggggtggtcataccctgtacccaggaaacaggcacggtagggctgagacagaagtcctgcttgtttccgcttatttatttgaaacaccgctcatttaggtcttactttgtttgccaggcactgttctaagctctgtataaatattaactcagagggtacaaatattaacttaagagttgttgcaggaaaaaaaataagcgcctctggctctttaagtttggcctccccctcaaaacccccgcaacggtcccaaaccccttccagggactgggactacggaccctggtccgaccttctcgcgggcttcccactgcgccaatcaaatcccagaaacagtgagtgctagaggcccggctgctaagcaacggcagagggcgggaagtttgaacgttctggacccgccccgaaggcaaataggccaatcagcgtccagactcttcagctacggcagtccgcttctcctcctcgccctgtcggatctctaggctggatccgggcctctccaatcaacagcggctaggagggcggggcgcgtgcgcgcgcacctcgctcacgcgccggcgcgctccttttgcaggctcgtggcggtcggtcagcggggcgttctcccacctgtagcgactcaggttactgaaaaggcgggaaaacgctgcgatggcggcagctgggggaggaggaagataagcgcgtgaggctggggtcctggcgcgtggttggcagaggcagagacataagacgtgcacgactcgccccacagggccctcagaccccttccttccaaagggtaacctccgcgtgacaggaatgagggtggggcgcgtggagtttcccacaatctgtactttagttaaatacccgagaattcacctcctgtgtccacagctctccacgcccctcagccctgccccgcagccctgtagcagaagtacttagtgctttgcattctgcgcgccaccctaccccggcctcctctgtgaatcgttgcttccgaaccgccctcactttttgcatccgcaga

SEQ ID NO: 69 - DNA, 2625 bp, Homo Sapiens
ttttaaacgagaagtgatgtttccggagcattaaaactgaagtgatttcaaaaccatgttgcactcacacgaacaggtgtgcacttaatggactaaactagttcagctgacatgtcttcttcattaggaacagtgtggagactgaaaaactaatttagcctagagcagctatttaattgtaaagtctcctttctcaaatattgatttactatgtgaggaaatatttactttgtatagaagtgtgtggaattggacgagggggttgacctacacatgtggtttggtatacacatatcctcattacagagggtgtaatgaagatataggtggttcagcaccataggaaagggaaaaaagaaaaaaaaaagacggtagaggtggcctcccaagcatccactcccactcctcttgttaatgattcacaatttgttgttattgttgtcatttactgttctccacacctttccacaaggcctgtgtgctttgaaaaaatatgtctctactccggatagaagtggggcacacagggccaggcgcggtggctcacgcctgtaatcccagcactttgggaggccgaggcaggcagatcacaaggtcaggagttcgagatcagcctggccaatatggtgaaaccccatctctactaaaaatacaaaaattagcctggcgtggtggcacgtgcctgtagtcccagatacttgggaggctgaggcagaagaatcacttgaacccgggaggcagaggttgcagtgagccgagatggtaccactgcactccagcctgggcgagagtgcaatgagactccgtctccaaaaaaaaaaaaaaaaagaaaaaaagaaaagtaagtggggcacacgattcaggcctaagctaaccagaccaacctcattcctgatggttgttaatgtttcagatacgggcccgcagccctacgtagagaagaggccaaggtagaaaacatgaatctgaggtaaaaagaaatgaggtacttgtttgcctcatcaagcctctcaattaaactaaccttgaagcctgtcttacctttggacttctagtgatgtcacccggtaaagcccatttgtttcaggacgtaagagttgggttttctgtgacttggaaccaaaaccattccaatttacaaaatgagcaactttaatattacccatgagaaatacttcattggtatatgctctttcctagcgtttttgaaaactaaactaggtgggtgaaaagtatatctttgcatgaaactttttcattccagaaaacattttgtcatcttgataataatggccaatgctactatatccaaatttttgtctttttttttttttgagacagagtctcgctctgccgctcaggtgtgatggcgcgatctcggctcactgcaacctctgcctccctggttcaagcgattctcctgcctcagcctccctgagtagctgggattacaggcatgcgccaccacacctggctaatttttgtatttttactgtagacggggtttcaccattttggccaggctggtctcgaactcccgacttccagtgatcctcctgcctacctcaaaaagcaacttgataaatccacaggctcggtatattttaaaaattcttttaaatacagtatacttttctctttttttccagaattaaccatgaatcgcacacacagccagaggcttttaacccgagaacggacaaaggggcctgcttgtgcaatacaattatttttaatggttaaacaaattaatacataagaccagctttacctaatataataataacgaaccaaagtttacaacagacaagaaaagcaccagctgtccccgccaccccggagcgatctccaaggggacgcgggagagcgccgcgggggacgcggaagtctgacgtcacaggaactgggggcggggcggggaggcccgcacaccctattgcgcatgctcccgcctccccggccgcggcctggcgcagtgcgcacgcgcgcgggtgggcgggtttgactggccgtagagtctgcgcagttggtgaatggcgttggtggcgggaaagttgagtctctcctgcgccgagccttcggggcgatgtgtagtgccttccatagggctgagtctgggaccgaggtgagagccgccgggttgggagtgagggagatgggaacaaggccgccggtgggcgaggggagccgagggaacccgggggattgggaggcttggggcggcgcggcctggccgggctgggaccggcctctcggcctagacgcccgcgatgctggcaccctctgccacctctcacctgggccccaggggtccgcccctgggcagcctggagtcctccgaggtgggaggaccgggcggaggtggaggaagtctttctttggaagacttgctgcctgcccagatcgatataacatacgaggtctctcctcccaagagttatggtctaaaaacccctcacaaattaactaccgttggaaatgtcaagctatgcaagaaaagctagaaaaggggaggggtcgcccgttggagcatttggagcttttctggaacaggtggtgtttgcggaggttgcctcacctccctgtagcccacgtgtctctgcttagggcagctggccctcgcca

SEQ ID NO: 70 - DNA, 2540 bp, Homo Sapiens
tagtcccagctactcgggaggctgaggcaggagaattgcttgaacccaggaagcagaggttgcagtgagctgagattatgccactgcactccagcctgggcaacagagggagactccatctcaaaaaaaaaaaaaatcattaaaatacagtaattcaggtttattaagtcattaccattgggttacctcacaaataaactaagtttagatgcgaactcaaagatactgagacactaatccatttcttaagctgctaagttagccttcttgaaacctcacttcgtagctctgcaaacaatgtacttttgacatcccaagctcacaggaataaaaaaccacctgccagttgtttccgttttccacctatgtctaatttatgtacttatatttataagaaacaaatcactaagtcttatttcatccttagttatgttgtgtttctatcgataacagcatgaagatttcggggacctggacattaaaataagtttgagtactggctttacaatctactaggtgtgatccgaggcaagtcagtctcttcatgtttcacttctttcacttgtaaacatctattcagaagttgctgtgaacttgatatttccatgcttataaactgattttttgaaaagagcctggtacataggacgtgataataaatgaaagcatttgctacttttggaaaaacaagcatgacaagatagtttatatactgttgatcttaagcacagtatatgcatcttatttttagctagtctgacagtgagataataaaaagagttatctttgacttgcactacgagtagaagaattcaacttcagtttctagaaagatgtataagaattaagagtggcagtcttcctagtctcaactgccatcttcccaccaggtggtaaattcgtccagagaagaaaatgaattattgctatatgggattctgcagcaacttctgtgaacataggctcataatttttcaccatggagactcaagctttttggagtcatagttgtttttgggtctatttgcaggcatgcatcctttgtccagaaatatacataacatttggcacatggacctggaggtaaaagaggaggaaggcctgaggctagacaccactccaataagtacattaagctcctagaagggcaatccacctttgcagagaactcttaactattaaaacctatagcttgtaaagcagcattttcaaagttaagagaagaaggtggaagggtcttgagaggctactgactaaacagatgaaaatgaaggtatggagtttggtgccaaaagaaactccccccaaaaatcaaacaataacaccagagtaaagcccctagggcgagataaggagttgcaacaaaacaagcggaaactcgagaagcgctaatgcttcaaagggtcaatgaccacacataatctacgtagccaacgtgttaaaacacaccaacgcatttttttttcctaaacaaagtaggaaagcggactttgcatgaggggcgggctgccgacccagcagtcttcctcggacagtccgtcctgattctctctggttggccgtggagggaccacatggctccaaggcctctcagctccgggcccacacaccccgggctgccgcacaaactccagccctagtctagatccacaaccccttctcgaagatcaaccgcgacctgggagccccacttcttaccatagcgaggccggcgatgccgcagccacatcacccttccggggctcaggcggaagaggctgcatgtcccgtctgcccttctcgccctctccagccgtccggttgggcttgtcacggcaccgcctaccaagacgggcggttaagacactaggataggctcctctccaccggaaaaggcgggatttagatcacgtcccgcaggccggcggaagtagctgatactctcattggttgcaaaaccttgatctgtgaaagcgggcgttttggaagataccggaagtagagtcacggagaggtaggatccggaagtggggctgcctctttaaataacaaaaatctgaggttctgttctttttatctttttgctttctttttaaaaaagttccctgctacttacccctagaactccacaatgcgagaatccccctcaatttgtgagctcccgcgacttcctcttgtgggcttttggggatgctagggttctcggcattatcctcagggtgcgacctgttcacccccttttcagtttctccgtttgcatctgagggattcttgggaatgcgaagcacttttgaaatgctctgtgttggttgtgggattgggaggacggttgaatccagagggtagtgttgagtaggctgtttgagcatttccccagcactggcctgtcctttcaatccccagatattggtaaactgtgggttccaaccaggcatcgaggctgaaacgtactaggcaatttgaggtcaggaaagaactttctgtggtaaccaatgggaaggaactgccgtttgcggactgcagcgattgattaggtactttaaagagatcaactggcaaga

SEQ ID NO: 71 - DNA, 2610 bp, Homo Sapiens
ctacaggctcgtgtcaccacactgggcaatacaaaaaatacaaaaaaaaaattttgtattttttgtagagacgaggtcttgccatattgcccaggctggaattcttacctttgttactgtatttaacgtatctttttcctccggccatcttcatggttttctctctgatttccacagtttgaatacactgcatgtgtcaggcaggggctcatatttatcaagttttgtgtgtgctctgagctcaggtctttcattattttgggaaaattattggtaattttctcttcaaacattttttatgatttgttctttcttcttcttttgggagtcctattacatgcatatgatatcatttgatattttcccacagttcttggatgctttttttaaaaaaaaactttttttcttctttattttccaacgtgggtaattcctatttttctcagctgtgttgatcctactgctgccccatcagaaaaattacctgttatcagcgttcttcctttcttataatttgatgagtttcctcctcatgcatattgttcacctttcgtacaagagacctccacatattaatcacagttaatttaaatttccagcctgtttcaatttctcgatcacctctgagtctagtcctgttaattgcttagtgttattttttgtttttgaaacagggtcttgctctgttgcccaggctggagtgcagcggcgcgatctcaggctgttccctgagttcacaccatccccctcaaccagcagattgcaaagtgtccgagtcgggccgtgcaggagtctttgtgggggtttcatggactccgaattctcatttctgctccatccccatctcatgaatccaaggccccactctgtgcctcggctcttcgtttgtggtgctgaacgtcatctacgtcatctacgccatctacgtaatcaacacaataaagacgcctgccgggaacgcggcccttcggctgaatcccttcggtggttccaaggccactgccagaggatgcggacgggtctccagggcctctacttacccaggactttgaggcacattagcttcgcctaggcactcgcttttacgaattcttatgtttggttttgttttgagacagagtctcgctctgccgcccaggctggttaaaagatagggtctcagccgggtgcggtggctcacgcctgtaatcccagcactttgggaggccgaggcgggcggatcacctgaggtccggagttcgagactagcctgggccaacatggcgaaacgctgtctctactaaaaataacaaaaatcatccaggcgtggtggcgcgcacctgcaatcccagctactcgggaggctgaggcaggagaatcacctgaacccaggaggcagacgttgcagtgagccgagatcgcgccactgcactccagcctgggcgacagagggagactccgtctcaaaaaaaggaaaaaaaaaaaaaagaaaagaaacaaaagtgatggggtctcgctctgttgcccaggctagtctggaattcctgggctcaagcgaccctccagcctcggcctcccaaagcgctgggaatacaggcgcggctaccgcgcggtctccggctgccgaaacaccgccctgcgcgcggaccgttcggccgccgggaggaacagcggctgcccggagctcagaggcgcgcgcggctttgcgctccccgcggcgctctgagcctgcctcggcttggttggccaggtggtctcttcaggaccaaccccagtcattcccggcaggaaccacgcttgaggggcggcagtctgcccgcgcgagacgcccccgcggactacaccgcggcggcaaagccaaacgcaaaaactacctcaccgcgcgcaggcgcctcccccaggaccaacatggccacgacgcaaggcctcgacctgaggggcgtggcctggccgccgccagccaacgggtgtgcgcgcctggccgcagccaataggaaggcagcgcgggctcgggcgcagggagccgccgccggggctgtaggcgccaaggccatgtccgactcgtgggtcccgaactccgcctcgggccaggacccagggggccgccggagggcctgggccgagctgctgggtaggtgggcgcggcaggccgcgggagtgggcggcgtccggcccgggacggtttcgccggttccccgatcccttcccgccagagcctccgccggtcggatccccggacgccgcgcccggggggctgtgcggggtgggcgcccggctggggcggcgcggctgcctcggacccggcccctcctgcgcctgggcggacgcccaccagaccgccgcccgcggggcgctcccttctttcccgaacgccgcccccgccggccgccctgtcaggcgggcctggggtgcgcggcctggggctcccctcagcgcagaggccgcccctcgccagccgtccccgggctcccctgcctcgggccctcctgggccgtcttccccggcgtccgcggtggggccgtctccgttagtttcccgagacctgcgccctggggaggagccccggcccctcttcgggagggtgtcgctggtgggtttctccgcggcgtccacctgcgcgtcgggccgggg

SEQ ID NO: 72 - DNA, 3076 bp, Homo Sapiens
gctgggattacaggcataacatggcccggccctggccatgtttttaactgtgtttctctaatagctaataatgccgagcatctttttatgtgtttcttagccattagtagatcttttttggtaaaatgtctttttttttttttttggtccatcttaaaattgttttttgttttgttttgagacagggtctcactttgttgcccacgctggagtgcagtggctcaatcatggctcactgcagcttcgacatccctgagctcaggtgatcctcccacctaagtttcccgagtagatgggactacaggtgtgtgccaccatgcccagctaatttttgtattttttttgtagaggtggggttttgctatgttgcccaggcaggtcttaaacttctgaggctcaaatgatcctcccacctcagcctcccaaagtgctgggataacaggcatgaaccaccacacccagctaagattgtttttaaaaatctttttcttgagttttgggagtttttatgtgttagggataccagtcccttatgaggtatataattagcaagtagtttctcccactctgtgactgtgacctttctttttttgaggcagggtctcactctgttactcaggctggagggcagtggtgtgatcatggctcactgcaacctggaactcctaggctcaagggctcctcccacctcagcctcccaagtagctgggtctacaggtgtgttattgtgccagggttaatgttttaaattttttgtagagataatgtctctacaaaagacaccatctttgttgcctaggctggtcttgaactcctggcttcagggaatcctccagcctcagcctcccaaagtgctgggattacagcatgagccacatccagcctatgatttttcttcttttcttttcttttctttttttttttttttgagatggagtctcgctgttgcgcaggctggagtgcagtggggcgatctcggctcactgcaggctctggcccgcggggttcacgcctttctcctgcctcagcctcccgagtagctgggactacaggcgcccgccacatcgcccggctaattttttgtatttttagtagagacggggtttcaccgtgttagccatgatggtctcgatctcctgacctcgtgatccgcccgcctcggtctcccaaagtgctgggatcgcaggcgtgagccacggcgcccggccccagcgtatgacttcttaatgatgtctttgtagtacaagagtttttaattttaataaagttaacttttttttaaattgtacaagcttttagtgctgtgtctaacaacttgttgccaaacccaaggtcataaagctgttctcttacgttttcttttttttttttttttgagacggagtctcactctgtcacccaggctggagtgcaatggcacgatgtcggctcactgcaacctccgccacccgggttcaagcgattcttccgcctcagcctccggggtagctgggattacaggcgcacgacaccacgccctgctaatttttgtatttttgtagagaaggtttcaccatgttagttaggctgctttacgttttcttttagaagttttatatttttggctcttatatttagtttgtgatccattgagttgattttatgtacgtatgtatggtcgcgttcttttctttcctgtcttttttttttttttttttttgcatatggatattcaattctcctagctccatttaatttgaaatgattgggcaggtacttttgagcagtgcaagtacagagcggcactgccagcagactacacgcggtagaaagccgaccttggtgagcgtgttggtgctcgacagtgagcagagaaaggatggacgattacggagcgccctcgtctccagttaccgctttctggaaacaccatccgccggggcggagctgttccgccccggtgcggtactacgactcccagcatgcacctcgcagtcggccctcggtggaagcgggaacccaggaggacctgggggtgtggcagcgaggaagggccgagccacggactgtggggccgaaactcgctcccgcccaccctttctcgaggctgtggcctccgcgagagccgagcgggccgcaccgccggccgtgcgactgccccagtcagacacgaccccggcttctagcccgcctaagcctgtttggggttgctgactcgtttcctccccgagtttcccgcgggaactaactcttcaagaggaccaaccgcagcccagagcttcgcagacccggccaaccagaggcgaggttgagagcccggcgggccgcggggagagagcgtcccatctgtcctggaaagcctgggcgggtggattgggaccccgagagaagcaggggagctcggcggggtgcagaagtgcccaggcccctccccgctggggttgggagcttgggcaggccagcttcacccttcctaagtccgcttctggtctccgggcccagcctcggccaccatgtcccgccagaccacctctgtgggctccagctgcctggacctgtggagggaaaagaatgaccggctcgttcgacaggccaaggtaacacggttgctggcaccctcggtttgcagcctcaagatccctgaaagcgggtttgcagtggatttaccccaacagatggggagggactgagcttgaccaaagagccagaaatgactggagaatgcatcccttgccactgctgcaaggggagaaaaaaggattgatcctcagtgacaacccctccctcatgtggcaggtggctcagaactccggtctgactctgaggcgacagcagttggctcaggatgcactggaagggctcagagggctcctccatagtctgcaaggtaggcgggtcctccccaggatggtcagttcccctcttccatagccagagaaacatccgctcctgcgtttttgggatcgatataattactcggggcagggagtcctgtttaaggcacagaggagactggagtggaatcatctttgtacaggcaaatccctctcttccttacacactcacagagtggcatttgaaaaatggtttccaagat

SEQ ID NO: 73 - DNA, 2567 bp, Homo Sapiens
cacaccatctcttgctccgtgagtatctttgtctctctagctcctcttcttctctcagtacatgtccctccttgactcccgcctctctgcaaggtgtatttggctgcctcagttggcctctccccctctgcatctctgggtggggtgttctctgcccgtctcccacccacacccacccccggtgctccccttccccccagcaggacagcggctcaggttcacgcaccccacggcgggccggctgggcgcacgcacgtccttgcacacaagccgcacgtagctgtacttgagcacgtcgatgagcgtgtagagcgggggcgcactggcccagcggcagcgcgccaggtgcatggagctcttgacgaagaagagcgccagccgctgctggcaccacgcgtcgaagaagcggctgaactcggcccacgagaagaaggcccgctcccgcagctcctgctcctcctgccccgcagccgtgccgggtgggggctccggccgctccatcctgggggcctgcgtggaggaggggagaacaggtggatatcagacccattcccacccggggtatctcatctactccattcttggcctgccccgtcggttgctggtgcctctatcgaggtgggtagcccggggtcggacgtgcctgtttttctccaaatatataaatatcaacctccatcctatctttggcctcctcccaccgccttatccctggttcacttggagcctgtcatcttgattcctaattccaactcgtctcctcctccgcagatgtgacccttaggtacagttggaatctctcctcccaaaatacgacccttaagctcagatgttccttaaggacatctcctcaaatgtgttctcaaattccagctaaaacctcctccccttccagctgtgtctctcacccaagagtaacttctaactctcgtattcatctggaactcctccttccatgtgccaacagttggctgtaacccctccaaagacgctccatctccagatgtgctcccacatccaggccacggacccctcacccggtcacatgcttcatgcacctgtggctccgcactccccagatgtgcctctggcgtgcagctgttgccccttcccccgattatgaccctatggctcgccacatgcagctgtagctggggcttccctgagacactctcatctccagatgtactccccacatgcagttatccacgcttcgcctacaggtgtgtgccccacttgtggctagttctcctcggaagtgtcaccagtattcacctgtggtcccctcctcctcagatgcggcccccagtccagctgtgggcccctcctcccagttacatccaccatcccccgcaatatgcatcttcgttctagacatggcccctcgtcctcggatgggctccttcaccccagatgctccccccacgtccagctgcgcgtctcccctcgagcagccccatccagcccgctcccgacgctcctactccccccctccccgcccgctgcggcaccttccagccccgccgtcccacctagctgtgcctctcccctccccaagatgtgcacccttcccgcccctccccactcacctacccgccccggagcggcgtccacctcccacaatgccccgcgcccaggcctggcccggcccttgctcccgggatgccccgcgcggtctcccgcctctcttcccgccgtgcctcgcgggggcgcttccaccgattcctcctctttccctgccagtcactcctcagaccctcagccacacccgctcatccagggcgagggaaagcgcgggcattttcccagtgtgctctgcgggagggctcgccccacttcaccccttttcccgccctcctcccattcgggagactacgactcccagtgtcctccgcgcgacggcggcggtgcggacggtgcccaggtcccgcccctaggctctgccccgcccccgcccgcagacgtctgcgcgcgaatgccgtggcgcgaacttgggactgcagaggcgcgcctggcggatctgagtgtgttgcccgggcagcggcgcgcgggaccaacgcaaggcaagtggggccgtccgcaagcagatgggaggcggagggcggcgggtgcgccgaatgcttggggcctatgcttcgccatgtcggggtgtctgcagaggagtgggcgtggggacgctgaggctgccgagagcgcggtggagacggaagagcgcgggctgcgggccgccggagagtgcagagaggtgtctcccagagggaggggggccaggtagagggtagacgagagacagagacagttggacaggtcctctgagaagaggccttgaggtgcgagttcacctggaagggggagaggccaaatggaactgaggggcggggcggggggggggaaaactgtgtgggcggggccagctggaaatcggaaggccccccgagggggcggggctatctgggagggggaggggctgaagggagctaaggggcggggccggggaaaagattgcgtgtgggcggggccacctggaagggggaggtgccaagggtggggctggctgggaaccggaa

SEQ ID NO: 74 - DNA, 2278 bp, Homo Sapiens
tcacagaagtcaaagctcaggaaaagcccctcgagggtttttgtgcggcagaggtgggttgtggggtgggattgtgcctgccacagtggaggggccctgcagacccagataaaccttcaagtggccagaagcgggggatggctctgctgggtgctggggctgccatgggccgtgggagccagcagtgtgcccagctccctcagggcccgtcccctaggcccttccgtccactgggccaagcaccgtccctgcccctccctaggggcatggatctgacttgagaggttgtgagagcttacaggcgctgggccgtcggggaggcctcagaagcgtaggacggctgcgcactgccgggccgtgttcagccctggtctggcctcggcctctagaggaggctgcctgcgctccagcaggcccaacccagaacgtgggcgagctcccttcagcatccctgggcggaaagagggatgggggctctgctgcagaggcagaatccgcgccgctccctccttccttcccccgaccagcctgtgacaaccccggccaggggcgggggcctccgcacaagcctggcgtccacttcctggataaggactccccggcccactccggaccagggctggggcggcctcccaggcgctcactccgctggcaccccaccggaaaacacgtctgcggcccgccccctcccccaaagcacgaccactccgcccgggcccctcgaggatccactcaggttcacgacgggcccgtcctctcggtggtctgaccaccggctggtggagtgggctctggggccgccaggcgaccagggcgcaggcgggggcggacagctcattgggaggggcgccggggcacagtgcggggctcgccccacccccaggtgccccttccccgctctcgcctcgcaggcaccgcatcgggcccgggaatcggtccggacctggcggtgggcgctgggaagaggatccacctccacgtggcccgccccgccccgggggcgcagccagttcccggcgctcactgccccccttctcccggcttccgtccccttctgcgcaggcgccgctccgccccggtcctaggggtgcttccgtggtcggcggctgctgggctccgcgccggggtccgagtcccacgaagccccggcccgagccgccggatgcccgcgcgcagcggggcccaggtgagcgcgcgcctcggccgccccgcggaacagacgcgcccacccccaggcgcagcagcgagcgcggccgcgggagcgggagtgccggggacgggcgtagcgcccaccgccccgagggttcggggcagagccagagcataggccaagggccaagctcgggccgagagcagtggccgcagcgcccgggggctgaacccacggcgcgctggcagcgcgggccgagctgcggagacggtcacgtcagcgtccgttccaggccgactggcagtctccgttctacattaacgtcagcactcccgttaaaaataatgcatctctcccatgccaggaggacttaggtgctgctaaagaccagccctccgggtgctgccaggccggcgctcacccgccaccttcatcttcccttctcctttgccccaggacagccgaggatgtgtggttaggttccccctacccatggggaggccagaggtgggaggctggcggcctgctcggtctcagcagaccctcctagtccctcaggagaccttgcctttgccccacttgctcgttatccagcctgggccatgaagcagaggacagttagggaccctgagcacgcggtggtcaccccggtgctcacccctccctgtgtgtccgaccttggccctgctaagatcctgtgttttgaattctggcaagggttggatgaaagggcagggctccagaaaccagctcagacgtttgcttgggacctgcatgatgagtgggaatcggagggcaccagccctgctgtcccaggctcaggcccccatctgctccccaggtcatgcagcctgggcccccatgccgtgcagctcgcacatatgtggggcagagcagccaccctgcccccagcagcagccgtccatcgtcagacgtgatcatttcctgaggcctcgagtgtgtcagggtgtttgtgcctcataacaacccacaggatggtcacccccgctttgcagatgaagaaaccaaagcaggtggtcagatccagtccttgcacttcctgagcctgaccttaccacacagctgtctcctattcggatgcttatttattttttttcccattacagt

SEQ ID NO: 75 - DNA, 2401 bp, Homo Sapiens
tcatgcctgtaatcctaacactttgggaagccaaggtgggaggactgcttgaggccaggagttcaatactagcctgggcaacacagcaagatctcatctctaccaagaaaaacaaaggatagaggagtcaactgaaaaagatcccagtgactaaagctcgaacaattttagcaataaaataaatacgcatgatataaatacatggctgaataaataaactggggagaatagaaaaatatcctgtgcagaagaattccaagtaacttatatagatattttacctttaccttcaaggaagtagaacataacttttcattccttcccaggatgggctaggcatgatgacttccttccaaagagtacagaacggaaacagggcagggggattaacagtggagaaacctgaccaacgctactgcagctaggtgatcaaggccaaaacatcgacagtgataaagcatgctgagagcacctttgatttgatgtagtgaaaatcgtgctttacctctgtaatcttcctgccaaaaacccataatcccagccccaattatgagagaaacattaggcaaatatcaattgagaaatattctacaaaatacctgactggtactcctgaaaactgtcaaggtcaccaaaaacaataaaagctcaagaaactgtcacagcccagaggaacctaagatgtgactactaaatggcatgtagtaccctaaatgggatcctggaacacaaaaagagtatcaggtaaaaactaagagaatcagaataaagaaaggacttttgttaataatagtgtatcaatattggttcatcaattttgacaagtgtaccatactaataatgcaaggtgttaataagaaacattcagcatgagatttttaggaattttctatattatcttcacaatttcctgttaatctaaatctctcctaatgacaagtttatttaaaaagtaaaacaaaacttgaaggagggaggaaacaagaagggaggaaacattggagacagaaccagcttggcaagttgacagataaggtctgagaagtaggcaggggaaagatcattcatttcaggcaatatttttccattttacctgtataagaaccatatgagccctatttttctttctttcttttttctttctttcttttcttttttttttttttttgtagagatgaagatttcactatgttgaacaggctggtctcaaactcctggcctcaagcaatcctcccacctcagcctcccaaagcatgagccaccatggtgggcctgtatgaaggaactttttaaaaaatgctacaagccgggtgcagtggctcattacctgtaatcccagcattctgggaggccaaggtaagaggatcacttgggcccagaagttcaagaccatcctgaacaacatagcaagaccctgttctctgcttaaaaaaaacaaaaacaagctgggcgtggtggatcacgcctgtaatcccagcactttgggaggctgaggtgggcagatcatgaggtcaggagttcgagaccagactgaccaacatggtgaaaccccatctctactaaaaatacaaaaattagctgggcacggtggtgtgcgcctgtgatcccagctactcaggaggctgaggcaggagaatcgcttgaacccgggagacggaggttgcagtgagctgagaaagcagtgagctgagatagcaccactgtgctctagcctgggagacggagtgagactctgtttcaaaaaaatcagcctgcccagtcagagcgcctcagcgccgtgctcgggacatcccgccctgcggccagcccccgcgtgacgtcaccgcattccggctccgctcctcccgccgcggcgcccgcaccgcagtgacagccagccgggcccggtgccggagaggaagtgcggtccgcgccaagcccgtccccgccgacgccggctccccgcggctcgggtgacagcgtcgcggccgccggacgcagcgcggggcaggcgcgggcagagccgagcgcagcggaggctccggcggaggcgcggggaaaatggctgatgactttggcttcttctcgtcgtcggagagcggtgccccggaggcggcggaggaggacccggcggccgccttcctggcccagcaggagagcgagattgcaggcatagagaacgacgagggcttcggggcacctgccggcagccatgcggcccccgcgcagccgggccccacgagtgggggtgagtcagcgcggggcctggagaggggctcagggcgcgcacccgggggaccccggccggggcccaggggcacagggaagagagcctgctctaggccacccggggcaggagctgggagacgtggggaagaatcttcttggagatctccatgtaggacttccgagctggggatga

SEQ ID NO: 76 - DNA, 2501 bp, Homo Sapiens
ccagcctgggccgcagagtgagaccctgtctcaaaaaaagaacctactagtctacataccacacttcctcatccccatctgagactatatatattttttctaacatgaggcaatgccaaaaagaggggctggtgagtgaaagtaagaacagaaagacatggaggcaagtcttatagaataatagccaacacttaaacttacacttaacagcgtgataggtattgttccaaacacattaaattcatttaatggtccttacatgtctatgtatttggtgattattatccttattattcacattgctgagtgtattattctgttctcatgatgctgatagagacatacccgagactggataacttattaaaaaaaaaaaggtttaatggactcacagttccacgtggatggggagtcctcacaatcatggtagaaagcaaaagacacgtcttacatggcagcagggaagagagagaaatgagaaccaaacaaaaggggtttccccttataaaaccatcagctctcatgcgacttattcactaccatgagaacagtatgggggaaaccacccccatgattcaatgatctaccaggtgcctcccacaacctgtgggaattatgggagctacaattccagatgagatttgggtggggacacagccaaaccacatcactgaggaaactgagttatagggagattagtaacgcccaacacagctggtaggtggtggagccaggcagtctgactctagggtctggactctgaactgcatcatgctgccaagaagttcctcattttttcctctctctaagtttcccttattcccctacagtcattccttcaacagcatttccttcaccatcttttctacttctactatataattaattttttcttcttggtcccaaattccaacgtgcaaatgcagccttatataccctaattcatctttacctttagactttcttccaatgtttctacttcattccattttaaatttatccatgagatgcctatttacaagctgtaaccatcatgaagtgaatgaagaataatacctactactgtacaatagaattccaagagtataaataggagttatggctttctgacttgaaactaaatacttgatacttgattttgctgtctgagatcaatctgaaaagtaataataatcactaacatttgttgagcatcaattgtgggccaagtgtcatttcaatcactctgtacatattaactcatttcatcctacaacaacccggtgaggcaagttctgttattctgttttacagttgaggaaacagaggcatagagagcttaagtagtttgcccagtagatagccagaagaggagccaggatgggtctcgggcagtttaacagcacagctgaagtcttaaccactatgccaacagctttttggtcctacacatcccatgggaagaggaaaataaaaaggtatctatttgtatacctttttatttctgatataagaagcagaattcctttcacatgacctatgtctatttaatacgtcattttgaaacttaccaataaaatttcccaagcgccagaaaactgttagtggctttttccatttctctctatttttttttgtgctactaattttgcttctttccctcagaaggctgccggaatagtaaacattcactgacatgtcataattactggaaaatgggcactggaaaatcacattgtaattaattcaaagcatgttttccaaatgtactactttaaattggagcttatatcataatccaaggaaacctttgtgtgtgtactgttcccacattgctcagcctgggatatccaggagtaattcaccttgcgcctgcctccagaccatcttccatggaagggggtgaccccttgcctcttggcaaccactatttctaagctgccaacattactcttgcattatcaacattctaacttcatgggaagggctgtggtgagtttctggaatgtgaataggaagttgtttttctaaacagcctgacactgaggggaggcagtgagactgtaagcagtctgggttgggcagaaggcagaaaaccagcagagtcacagaggagatggtgagtttatttttttctgcatgggaagtggttgaagtgagttggagtggtatggagtaaagtcaggcaggtaaaggttcagaaagtgaggaacagcgatagccatggagttttatgttgaattgcctattagattttgtgagtacttttaaacttgctgtccactttgaccctcccaacacccttgtgagttgaggttgctatttctattttacaaataaagccatcgtggtttacagaggctgtgttttatctaagcttcactgttaggctacatgatgttgggatctggggcctgtcctctggctccgcagctgctgttcctcctactagaatttataggggctctctgagaatagatcatggtaaacctgtcaccccattttccaagactgtacttctc

SEQ ID NO: 77 - DNA, 2501 bp, Homo Sapiens
cctgggtcctctcttccagctcccaaaatgtactctatttttatctgtttcacgaacgctggtccagatagtcttccatcccccactgactgttagaagtgactctcagcttttgtccatctcgaagtttctgtgctcagtgtgcctctcagactaaaggcttcctttgggaagccccgactctcgcttctcaggacagagatccaggggttgggggaggaaaaggttgaccagaagccatagcggagcagggagagagagtgtgaaagacagacccgcggccaggctcccagttctccagctcgtagagggcccaagtggccgctataatctgaaagagcagatatcgtaatcccatagtacttcctattggctgcaggacacagttctgtcctgacactgaaatttgggtgtgtcagggttctgggaattcacaacgctcacaacttgtgaagcagctgtggggtgggggatggggagggtttcagcagaggaagtgaggtcagtcaataattgatgcctgtctgagcttttagccattatctcccccagcctctattcctgtcaaaaggtggggcggggcaggaggaggggtccctggctcatcttgtagaatccccatattagagtaagacaccttagaggtctactcctgcttctaatacccacgtctttccaagtgtctctgaggccaccccctccccagccttttcatttattcatttaattaacgaacgccttcattgagggcctcctctgagtcaggctcagccagccagcatctttgctatgagctgagataagcatcatttccgtctattctcacaaccaccctatgaggctggcacggtttactatgcctatttagcagatgggggactgaagcatggagaggtgtcactagcctacggtaacacaaccagcctgcattcctagtaggtagtttgacttcagagtctctgtggataaccaggaggctaggactaagaccagagtcctgcaggtacttagatggttggagcaaagcagggcagtgaggtcagtgctcccagcctgtgcaggagcatcaggaagagtctgtgtccccctcccctgccggtatgaagccattctgcttccctccccagctgccttgtgtcagcagagttccagggaggctccattccccacctctatctaaagctccatttgctggggtgggggccctgcctggaaggggaaggtccaaggctgctcccagcgtgtccctccatcctgactgtccctggcggggcgggggtgtctttgtcacccagctgcacaacggccaggaagggctcaaaccatcctcagggctaacccaaggccgtcctctgggcctgtatacccctgtgctgagtgcggatcgggagaggctgctgaagacaggaggggacaaatgggggacgaaggggcccgagggaggggactgaaggatttgggccaagtcgggagttcccgagggcggagtcaaaacgcatctggattttgctagccccaaactctgccctcattgctgcaagcctcctagaccgaggacccccgggctgagggtggggtaaggataggtagtgtccctccccgtcccacccccgcctgtcccttcctcggtggccccttcccggcgccccgattccaggcggcccctccgctgctgccagccgatccccctctacccccacccactactccggccgccagacgttgcctacagtctcggctctgtctcccacggctgtgggtccggaccccacgggacccctatgggacccccacaggacccccacggcctgagtccaaggcccgccccctcggggaggcggatgtgggaggcccggcccgggtgcgggccagcgacccgggagctgcgggcggctgggaggggaggccgccctgaggggctgggagcggcgcgggggtgggtcccggtcctgcagccccagcgaggggcgagcggcggccagtcggcgagctgggcaataaggaaacggtttattaggagggagtggtggagctgggccaggcaggaagacgctggaataagaaacatttttgctccagcccccatcccagtcccgggaggctgccgcgccagctgcgccgagcgagcccctccccggctccagcccggtccggggccgcgcccggaccccagcccgccgtccagcgctggcggtgcaactgcggccgcgcggtggaggggaggtggccccggtccgccgaaggctagcgccccgccacccgcagagcgggcccagaggtgagtcgaggtccgcggacgggaccgggtggcgggcggcctgacccccgcttcagtgggcccttccttcgggcggaccccagagtcaccgcagagtggtcgcgggaggctcagtcccagctcattagaaaggcaagctgctcctggctgaccacgcacagctcccatgaccctacctgagacttggaggggaatggacgagactggactggaaatcagaaac

SEQ ID NO: 78 - DNA, 2501 bp, Homo Sapiens
tggctaattttttgtatttttagtagagacggggtttctccatgttgaggctagtctcgaactcctgacctcaggtgatctgcccgcctcagcctcccaaagtgctgggattacaggcgtgagccaccacgcctggccgctaactacatgtgttctatgaggtgaggtccttcccagaccctggaatcaggggttgcaattagggtccaaataatgaggttggactacagataacccatctcctttcttacctttgactagatccaaggactaaactccaagaacccgagcatctgtccccaaaactgaaaggattggactagtcaccccttgtttccctacagccacatcccaggcacctggcccttgctttgtccagaaattcagctataactccacacatctgatggccctttctggcaagcaggcatttccatcaggaccctcagctgccagacacatttactggaggtcacttattaaacctgggctcaatttccacacagggaggctactgaagcatcacactgggtctcccagccccttctcatagaggaaagatctctctgtcctgcagggttggcagtcagcgccaagtaaagggaatttagctcttggcccaagatccctgcccaggaaaggtacttgcgcctgctggaaactttgggctgaagtatactcctttccaaaaactcaggtctgatatttacacaaagtctgaaattaatgcagagaaaacttccaagtgcttggactggagcagaaggctgagaacaggaaggggctggtccctggtactagttttggttttttggtggtttttttttttcttgttttttctcacagaacagggcaaagctgagtgtccctggatgagtgaagcaggaggattaatcatgcccagtgcttctccactttaaactggttttcctgggaatttgcaattgagagtggggaggggtaagaatcgtgggaaaaggctgatggtgttcagccaaattcatccttcacgtgcccacccttctacaggcacatgctttggggccatccacggctgcagccaccccatccttaggaagcaccactggccttcctttccggtacctggactcagcatcactcccagcctcttggagatgcagccttcattcagcacacagctcagctctgagttctgtttttgtccctagatgtctctggggtcacctactactccctgcttggtggcccaggcccatccttctccactcttgcacctcttttagcagaaaaggagtgagaatggatatttccatgggccgtgtgtgcactcccggctacccctgacagctctactcagagctaccctccctcctggggcttcttatgtgttctaaggctgaggcaggaagactgtgagatcaggtgacactcaacagttatgatcggtcttaagattaacagtcctggccgggcgcagtggctcacgcctgtaatcccaacactttgggaggccgaggcaggcagaccacgagatcaggagatcaagaccatcctggctaacacagtgaaaccccgtctctactaaaaatacaaaaaattagccaggcgtggtggcgggcacctgtagtcccagctactcaggaggctgaggcaggagaatggcgtgaacccaggaggcggagcttgcagtaagccaagattgcgccactgcactcccgggtgacagagcgagactccgtctcaaaaaaaaaaacaacaacaacaacaaaaagattaacactccttctacttccaaacctaatacaaagggacattgcctagtgattaagagaattcattcattcaacaaatacttgttgagcacctactatgtgccaagcactgttctaggcaccggaaatacagcagtgagaaaaaccaaaaaaactccctgccctcatggggtgtatattcaagtagctgaaacagacagtgaacaaacaaaaaaggacaataatttcaaataataatgatgctatcggccaggtgtggtggctcatgcctataatcccagcattttgggaagccaagtcaagcggattacctgaggtcaggagttcaagaacagcctggccagcatggtgaaaccccatctctactaaaaatacaaaaattagccagacatggtggcacacacctgtaatcccagctacttgggaggctgacgcaggagaattgcttgagcccgggaggtggaggttgcagtgagccaagatctgacaggccttcagcaccactgcactctagactggctgacagagcgagactctgtcaaaaaaaaaaaagctataaatagactttaacagggtaacatgatagggagggagggataggggagcagggtggtcaaggaagggacatttaaacaggctagaatgacaatggccagcgagggaaagatccagaagtgtgtgctggaagaagaaagagcaagcacaaaacccttaggacaaaatcagctcgtgtggtcaaggcacagc

SEQ ID NO: 79 - DNA, 2501 bp, Homo Sapiens
tgtttctgacccctggctgcagcctaatgggccgactgctggacagcggtcctgagtcctgtttgaattggtgctgccccgacatcctctgacctcagctaatgatcctgcctgccgagggcagacaggtctctgcaaccctatgggtggtaggggtggtgatgagaggagaggtagtctcacttgcacagattttggtgtatggttctgtcttttgcactctttcaacagaggtctgtccagtccctcttgcaagtgtggggagggggtggtgcaggactatgaggtaactgtgagaagaggggctccagcagaaccagggtccaatggccttgaagagatggctggggacagctggactcattacgtctactcctaaatggaggaaacgacccctcagctacacagcacctgagccagaatgtcaccatggtgctgctccacaggatgacagctacctggtttgtgagggcccctattctagggacagctacttcattctgccctcccagagcagcaagcaacaaccctatgccaggaggccaattggcacgtcaagtgccagctccaatcgattgatagtagctgcctggctctgaaaggcagctgggatcgattcaccatgctgccagcacacagatggacccagcggtggtcccagcagtgagttcttgccttgggccatttcattttctttgtcctggccaaggaatgattggatgaacacactggactcccaatatgggtggataagacaagagtgtctggtcacacccctccaccactcataagcatggttgtgggcagtttggttccccaggcggccttggagaatgcaatgagccgaggaactggtcatctccaggtgcatccagggcaggaaaggatgacagcatgcgtgagccagggtcactggctaagaagtcatctcaggacctccccctagaaaagcccactgggcagcatccctgctggttcccccctacaccacaaggttacgcagagctggcggagggtcatggtcccactcatgtcaggtgctcttaatctggcaaggaaatgtaacctacgtgaatctcaacaggcagtgaagcaccgtttcttcctgactccaggtagggtgaagaaaatgggacagtagtacggggtgcgggcataaacgcacaactctgcctccccagacgcagagctgtggggctgtgagaatgccaggaggaggtaagaaagggcggccccatggggggcctgcagggtgggacaagcccaagaggtctctacatccaggcctggtgggggaggtgagcccctggtttaccgagggggtcccttcctgccctcggaaatactgcagctcctacctccatcgtctccccgctgcggggacccaggggcgtgaggatgagagagcccccaggccccagggtcagacgactgtgttcaagcaagtgagaacctctctgaggctgtttcccaactgtaaaatggggatagcagcagaactctctctcgcggcttgcgtgaagaatacaattcgatgtcgacaggagggagcggcgcgcagcgcgcagcgagtagcaggcgctgaagaaggatacctgtgaactgggagtggtggcggaggctacgcggccagagtccggggaaggggcgccggctctgccagtccctgctcggggctggatggtcgggggatgttctcgtaagtcggctgggagggagcggtcccgcgtaccctgccaccgccgccgcagaggttcgggcaggtgcggggccgcggcccctccgcgagggggccggtcatccgccgggactgacatcccggaggcccaatggcaagccgtcatctccgcgcatccgcccaatcggcgccggttgccgtgccgcgccgggtctctcgaccaatgggaaaatttgctgtcagatggggcggggcggagattcgcgtcgccggcccggtccgctttgcgcacgggccgcgtgagggcgggagggctggcccggggtctcgggttgcgcgctgggcctggagggagggggcggcccccgcaccggtccgagttgcggccgcgtggactgcgacccgcgccgcgccgcaccgcgccgcgccctgggaacgccgctccccgcgcgccaacggacccggggaagcccttctggggtccgaggccgcgctgcggggccgcccacgctgcgctccaggtaagcctgagccagtgggcggggtgtgggacccggggctggggcctcgggtcggagccgggactgggggcggggctgcagatatgggacgcattccgggcagcggtccggacagggtcctatccctggagtcgagatccgggcgagggtctgggccggacgtcggagccaatctccgccccacccgcgtcttgtccgcgcgctctgcggcgtccgagaccccgggccggcgggggcgggtctctttgtgcgtggccttggggccctaccctacccgtccgggcgtcttgcactgagcactc

SEQ ID NO: 80 - DNA, 2501 bp, Homo Sapiens
acagatgaccgaggggctcccagcccgggaggtggaaatccagcagggatttccaaggcctagtttgcagggctccaggatcgttcctagatcctggtcttgcagccttgacaaggggaaggagggaggcagcagaaggagggcagaacaatccatgccaggctgtgatttgccaagtgaccatctgggaagaatgggctctcagaccagggacagggagcagaggcaagcccgcatctgccctggttgcagaacccggattcagactcagggccccgatttctgcctggatcgctccactgggcggaggagtgactgtggacacatccagggttctctccaagtcggcttcctcatctgccaaatagagaccgcagaccaccagctcccaggcaggtgctactcttccggcccctcccaaggcaggagggccaggcgtactcgagacacaggtgtgctgggggcccaggtgggccagccagcagcatcctgcagggtaatgggagcaggtgggcaccccgaggctggcagtaaacactggctatctgcccccaggctcccaggaggggtcttgggcctcacctcctccggccggaacaggaaagcagctccaggcagctgggtccacaaaaatctccgttccctgaggtctcagaggcagtggcccaggagcatctggtcaccttcgggaaaaaccggcttggcaaaggctcccccgagggcacgcgtttcccggacagtgaggcaggacctaaactcttccgttaacactacatttttcgcatttctgcagtgtttgcactctcaggccccaccatttccccgcatctcttagggagaagttctcgacgtcccacctcccctggaagggtgctgctcccagagaccttcaggccaatggcccaatctcagtgccctcaggggagaggggggtgcagaaaaacagcctgggtcacaaaagaggtgcgagggctgtgagatcccggaggcaccgacgggaagcgagacggagaacaggagggcaggacgggctggaggtgggggatactgcagatggagggagccacggtgggggagggcgtggacctgaccgtcctggcacaaggcggtcgggtgcagacctccaggccctccgggttaaggtgccgcccagagccctcaggccgggggcgcacggaaaccacaggcagggtgcgcgtggagggacggggaaagcggggcgggttggggaaggcgccccgggaacctgaacctcccaccccgcctcagtctcgaccactccttaagccccaccccgccccaggtaaggcgcagtccacccccattcccagtagattaacgcacaggtgggggcgcgctcgggacatagctgcgctaggggacagcgcgcccagcccagtcgcgggggcgaggagcagggcggggcccagcaggaacccagctttgttagcgatgctccccgtgagccacgcgccacgcgtacgcgcttcctcaatggggccgggcgtggagccgcgccctgcgcgattggccaaacgggtggcccacgattggctgagaccctggcccccgcctcctcggccccaggagggtggggcgtgggtgtgggctgcgcggcgcgtgctgcccccggggatcttgcgcgcctcccgaacagccgtgttgtcgccagggccgcgccttccctcccacagcgcgcgctgcgcgtgcgaaggtctggcggctcttgggactggcggggctgcgcgcggggttagggtgggggtacgggaaggctcaacccaggacctgcgtaccttgctttgggggcgcactaagcacctgccgggagcagggggcgcaccgggaactcgcagatttcgccagttgggcgcactggggatctgtggactgcgtccgggggatgggctagggggacatgcgcacgctttgggccttacagaatgtgatcgcgcgagggggagggcgaagcgtggcgggagggcgaggcgaaggaaggagggcgtgagaaaggcgacggcggcggcgcggaggagggttatctatacatttaaaaaccagccgcctgcgccgcgcctgcggagacctgggagagtccggccgcacgcgcgggacacgagcgtcccacgctccctggcgcgtacggcctgccaccactaggcctcctatccccgggctccagacgacctaggacgcgtgccctggggagttgcctggcggcgccgtgccagaagcccccttggggcgccacagttttccccgtcgcctccggttcctctgcctgcaccttcctgcggcgcgccgggacctggagcgggcgggtggatgcaggcgcgatggacggcggcacactgcccaggtccgcgccccctgcgccccccgtccctgtcggctgcgctgcccggcggagacccgcgtccccggaactgttgcgctgcagccggcggcggcgaccggccaccgcagagaccggaggcggcgcagcggccgtagcgcggcgc

SEQ ID NO: 81 - DNA, 22 bp, Artificial Sequence
  primer
aatcctccaaattctaaaaaca

SEQ ID NO: 82 - DNA, 20 bp, Artificial Sequence
  primer
aggaaagggagtgagaaaat

SEQ ID NO: 83 - DNA, 22 bp, Artificial Sequence
  primer
ggataggagttgggattaagat

SEQ ID NO: 84 - DNA, 22 bp, Artificial Sequence
  primer
aaatctttttcaacaccaaaat

SEQ ID NO: 85 - DNA, 22 bp, Artificial Sequence
  primer
aaccctttcttcaaattacaaa

SEQ ID NO: 86 - DNA, 21 bp, Artificial Sequence
  primer
tgattgggttttagggaaata

SEQ ID NO: 87 - DNA, 22 bp, Artificial Sequence
  primer
ttgaaaataagaaaggttgagg

SEQ ID NO: 88 - DNA, 19 bp, Artificial Sequence
  primer
cttctaccccaaatcccta

SEQ ID NO: 89 - DNA, 18 bp, Artificial Sequence
  primer
tgtttgggattgggtagg

SEQ ID NO: 90 - DNA, 23 bp, Artificial Sequence
  primer
cataacctttacctatctcctca

SEQ ID NO: 91 - DNA, 22 bp, Artificial Sequence
  primer
ttttagattgaggttttagggt

SEQ ID NO: 92 - DNA, 22 bp, Artificial Sequence
  primer
atccattctacctcctttttct

SEQ ID NO: 93 - DNA, 18 bp, Artificial Sequence
  primer
ggaggggagagggttatg

SEQ ID NO: 94 - DNA, 22 bp, Artificial Sequence
  primer
tactatacacaccccaaaacaa

SEQ ID NO: 95 - DNA, 19 bp, Artificial Sequence
  primer
ttttgggaatgggttgtat

SEQ ID NO: 96 - DNA, 21 bp, Artificial Sequence
  primer
ctacccttaacctccatccta

SEQ ID NO: 97 - DNA, 22 bp, Artificial Sequence
  primer
ttgttgggagtttttaagtttt

SEQ ID NO: 98 - DNA, 22 bp, Artificial Sequence
  primer
caaattctccttccaaataaat

SEQ ID NO: 99 - DNA, 22 bp, Artificial Sequence
  primer
gtaatttgaagaaagttgaggg

SEQ ID NO: 100 - DNA, 22 bp, Artificial Sequence
  primer
ccaacaactaaacaaaacctct

SEQ ID NO: 101 - DNA, 20 bp, Artificial Sequence
  primer
ggagttgtattgttgggaga

SEQ ID NO: 102 - DNA, 21 bp, Artificial Sequence
  primer
taaaaccccaattttcactaa

SEQ ID NO: 103 - DNA, 22 bp, Artificial Sequence
  primer
tttgtattaggttggaagtggt

SEQ ID NO: 104 - DNA, 22 bp, Artificial Sequence
  primer
cccaaataaatcaacaacaaca

SEQ ID NO: 105 - DNA, 22 bp, Artificial Sequence
  primer
gatttttggagaggaagttaag

SEQ ID NO: 106 - DNA, 22 bp, Artificial Sequence
  primer
aaaactaaaaaccaaacccata

SEQ ID NO: 107 - DNA, 20 bp, Artificial Sequence
  primer
tggggttagtttaggatagg

SEQ ID NO: 108 - DNA, 25 bp, Artificial Sequence
  primer
cttaaaaacactaaaacttctcaaa

SEQ ID NO: 109 - DNA, 21 bp, Artificial Sequence
  primer
tttttgtattggggtaggttt

SEQ ID NO: 110 - DNA, 24 bp, Artificial Sequence
  primer
cccaactatctctctcctctataa

SEQ ID NO: 111 - DNA, 25 bp, Artificial Sequence
  primer
attagaagtgaaagtaatggaattt

SEQ ID NO: 112 - DNA, 19 bp, Artificial Sequence
  primer
tcaatttccaaaaaccaac

SEQ ID NO: 113 - DNA, 22 bp, Artificial Sequence
  primer
gggatgggttattagttgtaaa

SEQ ID NO: 114 - DNA, 22 bp, Artificial Sequence
  primer
ccttcacacaaaactacaaaaa

SEQ ID NO: 115 - DNA, 22 bp, Artificial Sequence
  primer
taattgaaggggttaatagtgg

SEQ ID NO: 116 - DNA, 22 bp, Artificial Sequence
  primer
aaaaccaaaaccaaaactaaaa

SEQ ID NO: 117 - DNA, 22 bp, Artificial Sequence
  primer
agtggatttggagtttagatgt

SEQ ID NO: 118 - DNA, 22 bp, Artificial Sequence
  primer
aacaaaataaaaacttctccca

SEQ ID NO: 119 - DNA, 22 bp, Artificial Sequence
  primer
taggggaaaagttagagttgag

SEQ ID NO: 120 - DNA, 18 bp, Artificial Sequence
  primer
cccattaacccacaaaaa

SEQ ID NO: 121 - DNA, 22 bp, Artificial Sequence
  primer
attttagtttgtgaaatgggat

SEQ ID NO: 122 - DNA, 21 bp, Artificial Sequence
  primer
tcttaaccaataacccctcac

SEQ ID NO: 123 - DNA, 22 bp, Artificial Sequence
  primer
gtgggttttgggtagttataga

SEQ ID NO: 124 - DNA, 20 bp, Artificial Sequence
  primer
taacctcctctccttaccaa

SEQ ID NO: 125 - DNA, 22 bp, Artificial Sequence
  primer
taggatggggagagtaatgttt

SEQ ID NO: 126 - DNA, 22 bp, Artificial Sequence
  primer
acaacttatccaacttccattc

SEQ ID NO: 127 - DNA, 22 bp, Artificial Sequence
  primer
tcccacaaaaactaaacaatta

SEQ ID NO: 128 - DNA, 21 bp, Artificial Sequence
  primer
aggttttagatgaaggggttt

SEQ ID NO: 129 - DNA, 23 bp, Artificial Sequence
  primer
tttggagggtttagtagaagtta

SEQ ID NO: 130 - DNA, 22 bp, Artificial Sequence
  primer
cccaataatcacaaaataaaca

SEQ ID NO: 131 - DNA, 22 bp, Artificial Sequence
  primer
atacaacctcaaatcctatcca

SEQ ID NO: 132 - DNA, 22 bp, Artificial Sequence
  primer
agggagaaggaagttatttgtt

SEQ ID NO: 133 - DNA, 22 bp, Artificial Sequence
  primer
ggaagatgaggaagttgattag

SEQ ID NO: 134 - DNA, 22 bp, Artificial Sequence
  primer
cctacaaccctatcctctaaaa

SEQ ID NO: 135 - DNA, 22 bp, Artificial Sequence
  primer
ttagtaggggtgtgagtgtttt

SEQ ID NO: 136 - DNA, 23 bp, Artificial Sequence
  primer
caaacaaaacttctatctcaacc

SEQ ID NO: 137 - DNA, 21 bp, Artificial Sequence
  primer
ttatagggttgagtttgggat

SEQ ID NO: 138 - DNA, 22 bp, Artificial Sequence
  primer
taaacaaacaacaaatcttcca

SEQ ID NO: 139 - DNA, 22 bp, Artificial Sequence
  primer
tgaaaatgaaggtatggagttt

SEQ ID NO: 140 - DNA, 22 bp, Artificial Sequence
  primer
ttaaaaccatataatccctcca

SEQ ID NO: 141 - DNA, 22 bp, Artificial Sequence
  primer
tatgtttggttttgttttgaga

SEQ ID NO: 142 - DNA, 22 bp, Artificial Sequence
  primer
aaccccatcacttttatttctt

SEQ ID NO: 143 - DNA, 22 bp, Artificial Sequence
  primer
gggtgtagaagtgtttaggttt

SEQ ID NO: 144 - DNA, 22 bp, Artificial Sequence
  primer
tttctccccttacaacaataac

SEQ ID NO: 145 - DNA, 22 bp, Artificial Sequence
  primer
tccccttccaactatatctctc

SEQ ID NO: 146 - DNA, 22 bp, Artificial Sequence
  primer
tgagagtgttttagggaagttt

SEQ ID NO: 147 - DNA, 22 bp, Artificial Sequence
  primer
aaaaccaaaacataaaccaaaa

SEQ ID NO: 148 - DNA, 22 bp, Artificial Sequence
  primer
gattaggagggtttgttgagat

SEQ ID NO: 149 - DNA, 21 bp, Artificial Sequence
  primer
aatggttgatgattttggttt

SEQ ID NO: 150 - DNA, 22 bp, Artificial Sequence
  primer
actctcttccctatacccctaa

SEQ ID NO: 151 - DNA, 24 bp, Artificial Sequence
  primer
tgttagtagagttttagggaggtt

SEQ ID NO: 152 - DNA, 22 bp, Artificial Sequence
  primer
acactacctatccttaccccac

SEQ ID NO: 153 - DNA, 22 bp, Artificial Sequence
  primer
tttttgtttttatggggtgtat

SEQ ID NO: 154 - DNA, 22 bp, Artificial Sequence
  primer
ttaaatatcccttccttaacca

SEQ ID NO: 155 - DNA, 23 bp, Artificial Sequence
  primer
agttagaagaggagttaggatgg

SEQ ID NO: 156 - DNA, 22 bp, Artificial Sequence
  primer
taattttccaatacccattttc

SEQ ID NO: 157 - DNA, 22 bp, Artificial Sequence
  primer
tgggtagtatttttgttggttt

SEQ ID NO: 158 - DNA, 22 bp, Artificial Sequence
  primer
cctaaaaactctctcatcctca

SEQ ID NO: 159 - DNA, 23 bp, Artificial Sequence
  primer
agtggtttaggagtatttggtta

SEQ ID NO: 160 - DNA, 22 bp, Artificial Sequence
  primer
aactccctccatctacaatatc

(19) United States
(12) Patent Application Publication
(10) Pub. No.: US 2007/0178453 A1
(43) Pub. Date: Aug. 2, 2007

(54) Method for amplification of nucleic acids of low complexity

(76) Inventors: Tamas Rujan, Berlin (DE); Armin Schmitt, Berlin (DE); Peter Adorjan, Berlin (DE); Christian Piepenbrock, Berlin (DE)

Correspondence Address: KRIEGSMAN & KRIEGSMAN, 30 TURNPIKE ROAD, SUITE 9, SOUTHBOROUGH, MA, 01772, US

(21) Appl. No.: 10/523,062

(22) PCT Filed: Aug. 1, 2003
(86) PCT No.: PCT/EP03/08602

§ 371 Date: Nov. 9, 2005

(30) Foreign Application Priority Data
Aug. 2, 2002 (DE) 102 36 406.0

(51) Int. Cl. C12Q 1/68; G06F 19/00; C12P 19/34

(52) U.S. Cl. 435/6; 435/91.2; 702/20

(57) ABSTRACT

The invention describes a method for amplifying nucleic acids, such as DNA with means of an enzymatic amplification step, such as a polymerase chain reaction, specified for template nucleic acids of low complexity, e.g. pre-treated DNA, like but not limited to DNA pre-treated with bisulfite is disclosed. The invention is based on the use of specific oligo-nucleotide primer molecules to solely amplify specific pieces of DNA. It is disclosed how to optimize the primer design for a PCR if the template DNA is of low complexity.

This invention relates to the fields of genetic engineering, molecular biology and computer science, and more specifically to the field of nucleic acid analysis based on specific nucleic acid amplification.

The matter of the present invention is a method for amplifying nucleic acids, such as DNA by means of an enzymatic amplification step, such as a polymerase chain reaction, specified for template nucleic acids of low complexity, e.g. pre-treated DNA, like but not limited to DNA pre-treated with bisulfite. The invention is based on the use of specific oligo-nucleotide primer molecules to solely amplify specific pieces of DNA. It is disclosed how to optimize the primer design for a PCR if the template DNA is of unusually low complexity. Also, for the optimal primer design it was considered that the treated template DNA is single stranded.

The amplification of nucleic acids relies mainly on a method called polymerase chain reaction (PCR). The PCR is based on the activity of the enzyme DNA polymerase, which is elongating primer molecules, which bind to the template DNA by adding dNTPs and hereby copying the template sequence (Saiki R K, Gelfand D H, Stoeffel S, Scharf S J, Higuchi R, Horn T, Mullis K B and Erlich H A (1988). Primer-directed enzymatic amplification of DNA with a thermostable DNA polymerase. Science 239: 487-491). The primer molecules are designed to specifically hybridize to those regions of the template DNA that define both ends of the amplificate. The forward primer binds to the 5′ end of the sense strand of the amplificate, whereas the reverse primer binds to the 5′ end of the reverse strand, hereby defining the starting points of the polymerase reaction and eventually determining the length of the amplificate.

Before the polymerase starts the template DNA gets denatured, this is usually done by a short cycle of heating the reaction mixture up to about 95° C., then cooling it down to the annealing temperature determined by the melting temperature of the primer molecules used and finally allowing the polymerase to elongate the annealed primers at its ideal working temperature for some minutes. This cycle is repeated several times each starting with the denaturation step. The primer molecules hybridize to the single stranded DNA. The forward primer is the starting molecule for a copy of the sense strand and the reverse primer is the starting molecule for a copy of the anti-sense strand.

These first copies will be of unspecific length, limited only by the polymerase's activity. However in the following cycle, the forward primer will also bind to the first copy of the anti-sense strand, the polymerase will take that copy as a template and will elongate the primer only as far as there is template DNA. Hereby the length of the second copy gets limited to the length defined by the first nucleotide of the second primer. In the following cycles more and more pieces of template DNA compete for the primer molecules and eventually the DNA amplificate of defined length will be the main product.

However, in the case of a bisulfite treated DNA the template DNA is single stranded. The bisulfite or similar treatment alters the original sequences on both strands such that these are not complementary to each other after the treatment. As a result no complementary strand to the target sequence exists. A first primer molecule binds to the one end of the single stranded target sequence. The polymerase elongates said primer and copies said target sequence. The second primer molecule cannot bind to the complementary, so called anti-sense strand, as it would in a standard PCR. Therefore the second primer molecule is designed to bind to the first copied sequence instead. More specifically it will bind to that part of the copied nucleic acid which is the complement to the other end of said target sequence.

The results of a PCR are highly depending on the choice of the ideal primer. The choice of a primer molecule must respect constraints permitting a correct amplification by PCR, fulfilling hybridization temperature conditions and auto- or hetero-hybridization prevention.

In other words, as any PCR requires two primer molecules to amplify a specific piece of DNA in one reaction the melting temperatures of both primers need to be very similar in order to allow proper binding of both at the same hybridization temperature. That is why most primer design programs require the user to define a preferred melting temperature or a permitted range of melting temperatures. This requirement becomes the limiting factor when designing primers for a so called multiplex PCR, as all primer pairs in use need to have the same or at least very similar melting temperatures. Additionally primers have to be very specific, in order to only amplify those pieces of DNA that are the target.

By providing the means for designing extremely accurate primer pairs for DNA hybridization procedures this invention relates to the so called PCR primer design. More specifically the body of this invention relates to the specific requirements of primers and therefore of primer design when using template DNA that consists of essentially only three different nucleotides and is single stranded. This is the case when using bisulfite treated DNA as a template, as it contains no cytosine other than the methylated cytosines in a CG dinucleotide and a rest of insufficiently treated and therefore untransformed non-methylated cytosines. The invention relates specifically to the primer design when using bisulfite treated DNA as template.

It would be obvious to an individual skilled in the art that the use of the primers as specified in this invention are not limited to nucleic acid amplification. Said primers can be used for several purposes, such as amplification, but also for nucleic acid sequencing or as blocking oligonucleotides during analysis of bisulfite treated DNA. Therefore the use of said primers is not limited to nucleic acid amplification but extends to all standard molecular biological methods.

Pairs of these primers are used to specifically amplify DNA from a small amount of sample DNA that consists of bisulfite treated DNA originating from a limited source of DNA like a bodily fluid or tissue sample.

DNA can occur methylated or non-methylated at certain positions and this information is relevant for the status of a genes transcription. The methyl group is attached to the cytosine bases in CpG positions. The identification of 5-methylcytosine in a DNA sequence as opposed to unmethylated cytosine is of greatest importance for example when studying the role of DNA methylation in tumorigenesis. But, because the 5-Methylcytosine behaves just as a cytosine for what concerns its hybridization preference (a property relied upon for sequence analysis) its positions can not be identified by a normal sequencing reaction. Furthermore in a PCR amplification this relevant epigenetic information, methylated cytosine or unmethylated cytosine, will be lost completely.

This problem is usually solved by treating the genomic DNA with a chemical leading to a conversion of the cytosine bases, which consequently allows to differentiate the bases afterwards.

A tool most useful for analyzing DNA methylation is the bisulfite conversion of DNA that converts cytosine bases into bases showing a hybridization behavior as thymin bases. Hereby the DNAs complexity is reduced by a fourth.

Bisulfite conversion is the most frequently used method for analyzing DNA for 5-methylcytosine. It is based upon the specific reaction of bisulfite with cytosine which, upon subsequent alkaline hydrolysis, is converted to uracil, whereas 5-methylcytosine remains unmodified under these conditions (Shapiro et al. (1970) Nature 227: 1047). However, in its base pairing behavior, uracil corresponds to thymine, that is, it hybridizes to adenine; whereas 5-methylcytosine doesn't change its chemical properties under this treatment and therefore still has the base pairing behavior of a cytosine, that is hybridizing with guanine. Consequently, the original DNA is converted in such a manner that methyl-cytosine, which originally could not be distinguished from cytosine by its hybridization behavior, can now be detected as the only remaining cytosine using “normal” molecular biological techniques, for example, by amplification and hybridization or sequencing. All of these techniques are based on base pairing which can now be fully exploited. Comparing the sequences of the DNA prior to and after bisulfite treatment allows an easy identification of those bases that have been methylated.

In the scope of this invention when it says “a nucleotide ( . . . ) was converted by the treatment . . . ” this conversion is meant to be able to differentiate between methylated and unmethylated cytosine bases within said sample, as for example the conversion of unmethylated cytosine bases to bases which hybridize to adenine by the treatment with bisulfite.

An alternative method is to use restriction enzymes that are capable of differentiating between methylated and unmethylated DNA, but this is restricted in its uses due to the selectivity of the restriction enzyme towards a specific sequence.

An overview of the further known methods of detecting 5-methylcytosine may be gathered from the following review article: Rein T, DePamphilis M L, Zorbas H, Nucleic Acids Res. 1998, 26, 2255.

In terms of sensitivity, the prior art is defined by a method, which encloses the DNA to be analyzed in an agarose matrix, thus preventing the diffusion and renaturation of the DNA (bisulfite reacts with single-stranded DNA only), and which replaces all precipitation and purification steps with fast dialysis (Olek A, Oswald J, Walter J (1996) A modified and improved method for bisulfite based cytosine methylation analysis. Nucleic Acids Res. 24: 5064-6). Using this method, it is possible to analyze individual cells, which illustrates the potential of the method.

To date, barring few exceptions (e.g., Zeschnigk M, Lich C, Buiting K, Doerfler W, Horsthemke B (1997) A single-tube PCR test for the diagnosis of Angelman and Prader-Willi syndrome based on allelic methylation differences at the SNRPN locus. Eur J Hum Genet. 5: 94-8) the bisulfite technique is only used in research. Always, however, short, specific fragments of a known gene are amplified subsequent to a bisulfite treatment and either completely sequenced (Olek A, Walter J (1997) The pre-implantation ontogeny of the H19 methylation imprint. Nat Genet. 3: 275-6) or individual cytosine positions are detected by a primer extension reaction (Gonzalgo M L and Jones P A (1997) Rapid quantitation of methylation differences at specific sites using methylation-sensitive single nucleotide primer extension (Ms-SNuPE). Nucleic Acids Res. 25: 2529-31; WO 95/00669) or by enzymatic digestion (Xiong Z, Laird P W (1997) COBRA: a sensitive and quantitative DNA methylation assay. Nucleic Acids Res. 25: 2532-4).

Another technique to detect hypermethylation is the so called methylation specific PCR (MSP) (Herman J G, Graff J R, Myohanen S, Nelkin B D and Baylin S B (1996), Methylation-specific PCR: a novel PCR assay for methylation status of CpG islands. Proc Natl Acad Sci USA. 93: 9821-6). The technique is based on the use of primers that differentiate between a methylated and a non-methylated sequence if applied after bisulfite treatment of said DNA sequence. The primer either contains a guanine at the position corresponding to the cytosine in which case it will after bisulfite treatment only bind if the position was methylated. Or the primer contains an adenine at the corresponding cytosine position and therefore only binds to said DNA sequence after bisulfite treatment if the cytosine was unmethylated and has hence been altered by the bisulfite treatment so that it hybridizes to adenine.

With the use of these primers amplicons can be produced specifically depending on the methylation status of a certain cytosine and will as such indicate its methylation state. The present invention, however, does preferably not include CpGs in the primer sequence.

Another new technique is the detection of methylation via Taqman PCR, also known as MethylLight (WO 00/70090). With this technique it became feasible to determine the methylation state of single or of several positions directly during PCR, without having to analyze the PCR products in an additional step.

In addition, detection by hybridization has also been described (WO 99/28498).

Further publications dealing with the use of the bisulfite technique for methylation detection in individual genes are:
Grigg G, Clark S (1994) Sequencing 5-methylcytosine residues in genomic DNA. Bioassays 16: 431-6; Zeschnigk M, Schmitz B, Dittrich B, Buiting K, Horsthemke B, Doerfler W (1997) Imprinted segments in the human genome: different DNA methylation patterns in the Prader-Willi/Angelman syndrome region as determined by the genomic sequencing method. Hum Mol Genet. 6: 387-95; Feil R, Charlton J, Bird A P, Walter J, Reik W (1994) Methylation analysis on individual chromosomes: improved protocol for bisulphite genomic sequencing. Nucleic Acids Res. 22: 695-6; Martin V, Ribieras S, Song-Wang X, Rio M C, Dante R (1995) Genomic sequencing indicates a correlation between DNA hypomethylation in the 5′ region of the pS2 gene and its expression in human breast cancer cell lines. Gene 157: 261-4; WO 97/46705; WO 95/15373; WO 97/45560

For all those methods mentioned above, which are based on PCR amplification of bisulfite treated DNA, the biggest challenge is to design primers that are specific.

### THE PROBLEM AND ITS SOLUTION

There are a number of programs available on the market that offer to design primer pairs in order to amplify a piece of DNA in a PCR. Usually they require as input the template DNA sequence, the preferred melting temperature TM, the desired length of the amplificate and optionally the preferred length of the primer molecules.

However if a primer is required to bind specifically to bisulfite treated DNA, the design of the primer molecule is especially difficult and those tools known in the art are not competent to design primers that lead to specific products. The following problems occur when dealing with bisulfite treated DNA instead of standard DNA:

First, the sequence complexity of the bisulfite treated genome is reduced dramatically. Complexity in this context is meant to be a measure for the similarity of a given sequence to a random or stochastic sequence; the more complex a sequence is the more it is similar to a random sequence. A reduced complexity of the genome means there are less degrees of variation. Where there are essentially only three different nucleotides rather than four, the probability of a sequence to occur twice in a given length of sequence is much higher. For example, a primer molecule of 20 nucleotides in length is likely to be unique in the human genome, if it is not part of a repeat sequence: The human genome is known to consist of about 3×109 bases. There are 420≈1012 different ways to form sequences of a length of 20 nucleotides, assuming equidistribution of the bases, which makes multiple occurrences of a given 20-mer (oligonucleotide of 20 nucleotides) extremely unlikely. However since there are only 320≈3×109 different 20-mers possible over a 3-letter alphabet, this multiple occurrence cannot be excluded. In addition a bisulfite treated sequence, enriched in thymine in the sense strand and enriched in adenine in the reverse complementary strand, will contain more repeats and regions of general low complexity.

Another way to enhance or guarantee uniqueness of primer and/or oligo molecules is to estimate their expected frequency in the genome based upon a Markov model of order n for the human genome or to check their uniqueness explicitly by counting their exact occurrence. The estimation based upon the Markov model relies upon the determination of the probabilities of all 4n n-mers (oligo molecules of n nucleotides) in the human genome or in all amplificates which are used in the hybridization and the conditional probabilities of all four bases given these n-mers. The primer pairs will be constructed from forward and reverse oligos which lie within an appropriate distance to each other and which have minimal individual expected occurrence elsewhere in the genome.

A second challenge in primer design for bisulfite treated DNA is that the melting temperature TM of a bisulfite DNA primer of a certain length is typically lower than the melting temperature TM of a standard primer containing cytosines. This is due to the fact that every cytosine in a bisulfite treated DNA is—after amplification by PCR—replaced by thymine. Cytosine binds its corresponding base guanine via three hydrogen bonds, whereas thymine binds its corresponding base adenine via two hydrogen bonds only, leading to a generally weaker binding, a lower TM.

A third problem arises from the fact that bisulfite treated sequences are not only lacking cytosines but are also thymine-rich. Thymine also hybridizes unspecifically with guanine. This makes mismatching (unspecific binding of a primer to a sequence not identical) of a primer designed for bisulfite treated DNA much more likely than mismatching of a standard primer consisting of four different nucleotides.

It is the aim of this invention to overcome these problems, which are specific for primer based amplification of bisulfite treated DNA.

For a so called “multiplex PCR” it becomes especially difficult to design primer pairs. This expression is used to describe an experiment in which several different pieces of DNA are amplified simultaneously, in one reaction vessel and at the same time. Obviously this saves a lot of effort and time and is as such a basic requirement for high throughput assays based on PCR amplification. An overview on the state of the art concerning multiplex PCR is given by Henegariu et al. (Henegariu O, Heerema N A, Dlouhy S R, Vance G H and Vogt P H (1997) Multiplex PCR: Critical Parameters and Step-by-Step Protocol. BioTechniques 23: 504-511), who offer a step-by-step protocol on how to tackle multiplex PCR problems. However, the possibility of a special primer design is not mentioned in this article.

To ensure that the multiplex PCR works and the multiple products are amplified indeed usually a gel electrophoresis of the reaction mixture is performed. The products get separated due to their different sizes. Unfortunately, the ability of agarose gel electrophoresis to distinguish the products is slightly limited. However, it is possible to test for different product sizes with the means of a fragment analyzer, which is much more accurate and able to distinguish product sizes of one base difference. Hence different product sizes are no longer a requirement to be considered in the primer design for a multiplex PCR.

In patent WO 01/94634 a method for a multiplex PCR using at least two primer pairs is described that consists of basically a two step amplification procedure wherein one step is referred to as pre-amplification. After pre-amplification (by means of PCR) with a number of primer pairs the sample gets divided into as many portions as there are primer pairs. At least one (and preferably only one) of the previously used primer pairs is added. This method doesn't relate in any way to the selection or design of primer molecules described herein.

In an article by Shuber et al. (Shuber A P, Grondin V J and Klinger K W (1995) A simplified procedure for developing multiplex PCRs. Genome Res 5 (5): 488-493) regarding multiplex PCR, the authors suggest to use primers, which contain a 3′ region complementary to sequence specific recognition sites and a 5′ region of a defined length of 20 nucleotides each. The authors claim that they could establish identical reaction conditions, cycling times and annealing temperatures for any PCR primer pair following those requirements.

In several recent papers successful multiplex PCRs have been established. For example, Becker et al. have reported the development of a multiplex PCR reaction for the detection of multiple staphylococcal enterotoxin genes, which uses individual primer sets for each toxin gene (Becker K, Roth R and Peters G (1998) Rapid and specific detection of toxigenic Staphylococcus aureus: use of two multiplex PCR enzyme immunoassays for amplification and hybridization of staphylococcal enterotoxin genes, exfoliative toxin genes, and toxic shock syndrome toxin 1 gene. J. Clin. Microbiol. 36: 2548-2553). This has been developed even further by Monday and Bohach, by increasing the number of primer pairs applied in one reaction up to about 10 in order to have one assay to amplify all of the characterized enterotoxin genes. This still required a unique established primer pair for the detection of every individual gene (Monday S R and Bohach G A (1999) Use of multiplex PCR to detect classical and newly described pyrogenic toxin genes in staphylococcal isolates. J. Clin. Microbiol. 37: 3411-3414).

In another paper by Sharma et al. a method for a one-vessel-multiplex PCR is described wherein each of six chosen primer pair consists of one identical universal forward primer, based on a highly conserved region of those genes of interest and one reverse primer, specific for each individual gene. As such the assay leads to a rapid amplification of a family of genes, which all have a conserved region in common. It is designed to detect presence or absence of certain genes in an unknown mixture. No further information is given about the primer design, apart from saying that they were designed by alignment of published DNA sequences. This is certainly not the only requirement though, as one big limitation of the method is the need of getting PCR products of different sizes in order to identify those in the end (Sharma N K, Rees C E D and Dodd C E R (2000) Development of a single-reaction multiplex PCR toxin typing assay for Staphylococcus aureus strains. Applied and Environmental Microbiology 66 (4): 1347-1353).

In the patent application WO 01/36669 a method is described which uses a similar approach for the controllable amplification of a higher number of sequences in selecting one randomly chosen reverse primer that hybridizes unspecifically and a number of specific forward primers to amplify a group of sequences. As the reverse primer is labeled all products formed will be labeled as well. By hybridizing said amplicons towards immobilized detection oligos, which are able to differentiate the products, it will be easy to see which products have been amplified and herein the presence or absence of said sequences in the mixture can be determined.

The big disadvantage in all these methods is that every primer pair needs to be established individually first to ensure that a PCR product of the expected size was produced and that no additional or nonspecific products are generated. Once the specificity of the primer pairs had been determined, PCR conditions, buffers, and primer concentrations need to be optimized to establish conditions under which the primer molecules can be combined into one single PCR reaction without affecting the ability of the primer pairs to generate a gene specific amplicon.

A more recently published approach by Nicodeme and Steyaert describes the conditions required for multiplex PCR and suggests an algorithm to automatically select for primer pairs (Nicodeme P and Steyaert J M (1997) Selecting optimal oligonucleotide primers for multiplex PCR. Proc. Int. Conf. Intell Syst Mol Biol; 5: 210-213). In this approach the conditions for pre-selecting primer pairs for a successful one locus amplification (singleplex PCR conditions) are rather broad. The three basic requirements are the pairing distance between a forward and a reverse primer, the condition of non-palindromicity of a primer, and the condition that the 3′ end of a primer must not be reverse complementary to any of the other primers sequence. This selection is done with the help of a typical primer design program called PRIMER. However, PRIMER is a two step program, and in this approach the new method to design primers for a multiplex PCR takes the output from step 1 as input, which is a list of possible forward and a list of possible reverse primers for every amplificate.

The only further selection criteria for the multiplex PCR primers are the absence of the reverse complementarity of their 3′ end towards the other primer sequences in the experiment. A second critical factor considered here is the GC versus AT ratio. To some extent it is this ratio that determines the melting temperature of a primer pair. The authors suggest to limit the GC/AT ratio to be inside a given range which would enable the simultaneous hybridization of several primer pairs at one reaction temperature. The final requirement is the electrophoresis distance, determined by the tool that is used to differentiate the PCR products in, for example, a gel electrophoresis. This most common method requires the products to be of different sizes. The whole concept of this method also requires to have a pool of possible primer pairs for each amplicon.

The design of suitable primers for a multiplex PCR on bisulfite treated DNA is an even greater challenge. The low complexity of the DNA, being reduced to essentially three different bases rather than four different bases, requires an extra careful selection of primers to avoid mismatching and unwanted amplification.

In the scope of this invention the word “mismatching” corresponds to the situation when the alignment of two sequences which are essentially complementary reveals positions in one of the sequences where the nucleotide base does not align with its corresponding base but a different one. The corresponding or complementary base pairs are adenine and thymine, cytosine and guanine, are adenine and thymine, cytosine and guanine, uracil and adenine. For example, a cytosine that aligns with a thymine in its otherwise complementary sequence creates a mismatch of one base or nucleotide.

Accordingly “base mismatches” refers to the situation of a base mismatching with another as explained above, respectively “one or more base mismatches” refers to one or more bases (in a given sequence) that cannot be aligned with their corresponding bases.

Also, when the alignment reveals single nucleotide gaps in one of the aligned sequences this is understood under the term “mismatch” in the scope of this invention.

A ‘gap’ is to be understood as follows: If an alignment reveals that, in order to get the highest number of corresponding base pairs aligned, some bases are lacking a corresponding base in its otherwise complementary sequence, this is called a gap. Such a gap can have a length of one or more nucleotides.

To solve the problems mentioned above we invented a method consisting of several steps that is applicable for the amplification of nucleic acids in singleplex as well as in multiplex PCR experiments.

### SUMMARY OF THE INVENTION

The method is comprised of the following steps:

Firstly, the nucleic acid sample containing the region of interest, which is to be amplified, is isolated. Secondly, this nucleic acid sample is treated in a manner that differentiates between methylated and unmethylated cytosine bases within said sample. Thirdly, a reaction mixture is set up containing a) the treated template nucleic acids, carrying the region of interest (also called: target nucleic acid) that is to be amplified, b) specified oligo-nucleotide primers, c) an enzyme capable of amplifying said nucleic acids in a defined manner, d) the necessary nucleotides required for the nucleic acid synthesis and e) a suitable buffer.

Said specified oligo-nucleotide primers are characterized in that their sequences each reach a predefined measure of complexity (as described in detail below) every possible combination of two primer molecules in said reaction mixture has a melting temperature below a specified threshold temperature none of the possible combinations of two primer molecules in said reaction mixture leads to the amplification of an additional unwanted product as determined by virtual testing for amplification.

In the last step of the method said amplified target nucleic acid is detected by means commonly used by one skilled in the art.

The invention is composed of a method for the amplification of nucleic acids comprising the following steps of isolating a nucleic acid sample, treating said sample in a manner that differentiates between methylated and unmethylated cytosine bases within said sample, amplifying at least one target sequence, within said treated nucleic acid, by means of enzymatic amplification and a set of primer molecules, wherein said primer molecules are characterized in that
a) each primer molecule sequence reaches a predefined measure of complexity, b) every combination of any two primer molecules in the set has a melting temperature below a specified threshold temperature and c) every combination of two primer molecules, under conditions allowing for one or more base mismatches per primer, does not lead to the amplification of an unwanted product when virtually tested using the treated and the untreated sample nucleic acids as template and the last step of detecting said amplified target nucleic acid.

### More Detailed Description of the Method:

The method is comprised of the following steps:

In the first step of the method, the nucleic acid sample, which contains the region of interest that is to be amplified, must be isolated from tissue or cellular sources. Such sources may include at least one cell, but usually several cells, cell lines, histological slides, bodily fluids, or tissue embedded in paraffin.

In a preferred embodiment of this invention the nucleic acid sample is isolated from a bodily fluid, a cell culture, a tissue sample or a combination thereof.

For example a certain kind of organ sample from a patient or an animal can be used to extract genomic DNA by the usually applied methods. Preferably, in this invention DNA is extracted from a tissue sample or a biological fluid like blood, serum, urine or other fluids. ‘Bodily fluid’ herein refers to a mixture of macromolecules obtained from an organism. This includes, but is not limited to, blood, blood plasma, blood serum, urine, sputum, ejaculate, semen, tears, sweat, saliva, lymph fluid, bronchial lavage, pleural effusion, peritoneal fluid, meningal fluid, amniotic fluid, glandular fluid, fine needle aspirates, nipple aspirate fluid, spinal fluid, conjunctival fluid, vaginal fluid, duodenal juice, pancreatic juice, bile and cerebrospinal fluid. This also includes experimentally separated fractions of all of the preceding. ‘Bodily fluid’ also includes solutions or mixtures containing homogenized solid material, such as feces.

The nucleic acids may include DNA or RNA. Isolation may be by means that are standard to one skilled in the art, this includes for example extraction of DNA with the use of detergent lysates, sonification and vortexing with glass beads. An example is the extraction of DNA from a piece of a plant, like a leave or fruit. Once the nucleic acids, like genomic double stranded DNA, have been extracted they are used in the analysis.

In a preferred embodiment of this invention the nucleic acid sample is comprised of plasmid DNA, BACs (bacterial artificial chromosomes), YACs (yeast artificial chromosomes) or genomic DNA.

In another especially preferred embodiment of this invention the nucleic acid sample is comprised of human genomic DNA. It is preferred that the nucleic acids are of human origin.

In the second step, this nucleic acid sample is treated in a manner that differentiates between methylated and unmethylated cytosine bases within said sample. Cytosine bases which are unmethylated at the 5′-position are converted to uracil, thymine, or another base which is dissimilar to cytosine in terms of hybridization behavior. This will be understood as ‘treatment’ hereinafter. The method most commonly used so far is the so called bisulfite treatment.

This step is of essential meaning to the process as it translates the methylation pattern of said nucleic acids into a pattern that is something like an imprint of the methylation status itself. It contains essentially the same information but the pre-treated nucleic acids are no longer sensitive to amplification via PCR. Amplification via PCR does not differentiate between methylated and unmethylated cytosines and therefore leads to the loss of this level of information. The original methylation status however can be deducted whenever the described pre-treatment had been performed prior to the amplification step. Hence any means suitable to differentiate between a methylated and an unmethylated cytosine base are applicable, as long as the modified bases are still capable of being amplified by enzymatic means after treatment.

It is a preferred embodiment of this invention that said sample is treated by means of a solution of a bisulfite, hydrogen sulfite or disulfite. A treatment of genomic DNA as described above is carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior.

In the third step of this method, a reaction mixture is set up containing a) the treated template nucleic acids, comprising the region of interest (also called target nucleic acid) that is to be amplified, b) specified oligonucleotide primers, c) an enzyme capable of amplifying said nucleic acids in a defined manner, for example a polymerase, d) the necessary nucleotides required for the nucleic acid synthesis and e) a suitable buffer. The template nucleic acid contains at least one target nucleic acid, which is amplified in the reaction. One primer molecule of the at least one primer pair in the reaction mixture is capable of binding to the 3′ end of one specified target nucleic acid. The first primer binds to the 3′ end of the target sequence, this primer is elongated and a complementary sequence to the target sequence is made. The polymerase stops to elongate unspecifically. The next cycle starts by thermally denaturing the now double stranded template nucleic acid into single stranded template nucleic acids. This is followed by the next phase of annealing when both primer molecules specifically bind to the target nucleic acid and its complementary strand. The second primer is identical to the 5′ end of the target molecule. It doesn't bind to the target sequence itself but to said complementary nucleic acid to the target sequence, as soon as this is denatured from the template.

The process is finished by the actual amplification phase at a slightly lower reaction temperature, during which the enzyme, for example the polymerase elongates the primer as a complementary sequence to the target nucleic acid. The polymerase elongates this second primer by using the first copy as template until the end of said copied nucleic acid is reached. That way an identical copy to the original single stranded target nucleic acid is created. Hence, the length of the amplificate is determined by choosing the two primers.

The elongation products, being complementary to each other and hereby building a double stranded version of the target nucleic acid, serve as additional targets for the primer molecules binding in the next cycle of amplification.

Essentially step 3 of the method is comprised of amplifying at least one target sequence, within said treated nucleic acid, by means of enzymatic amplification and a set of primer molecules.

Said primer molecules used in said method are characterized in that they, in addition to fulfilling all the usual requirements towards a PCR primer as will be specified in more detail later, also fulfill the following requirements:

Firstly, the sequence of each primer molecule used in step 3 of this method reaches a predefined measure of complexity.

In a preferred embodiment of this method the primer molecules are reaching a certain value of linguistic complexity. A notion and a measure of linguistic complexity has been introduced by Trifonov in 1990 and has been used for analysis of nucleotide sequences before (Trifonov, E N (1990) Making sense of the human genome. In Structure & Methods. Vol 1 pp 69-77 (eds. Sarma, R H and Sarma M H, Adenine Press, Albany, US). The linguistic complexity technique allows a calculation to be made of the structural complexity of any linear sequence of characters irrespective of whether the text is cognized or presently undeciphered. The sequences are compared exclusively from the point of view of their structural complexity with no reference to the meaning of the texts. In 1997 Trifonov published how the linguistic complexity of nucleosomal sequences is defined (Bolshoy, A; Shapiro, K; Trifonov, E and Ioshikhes I. (1997) Enhancement of the nucleosomal pattern in sequences of lower complexity. NAR 25 (16): 3248-3254). Quote: “The linguistic complexity measure exploits the major distinguishing feature between natural nucleotide sequences and uniformly random ones: the repetitiveness of the natural sequences, i.e. the frequent repetition, not necessarily a tandem one, of some oligonucleotides (“words”), while others are avoided. ( . . . ) Complexity can be directly calculated as the extent to which the maximal possible vocabulary (all word sizes considered) is utilized in a given strength of sequence ( . . .).

In another preferred embodiment of this method said measure of complexity is set by the so called Shannon entropy (Shannon, C E, (1948) A Mathematical Theory of Communication, University of Illinois Press, Urbana). This is the most common measure to assess the information content (in a technical, non-semantic meaning) of linear information carriers. It attributes the maximal value (which can be chosen to be 1 without restrictions) to sequences where all symbols (characters) occur at equal probability and a value of 0 to sequences consisting of just one repeated symbol (character, letter). A derived and more general measure is the higher order Shannon entropy which attributes maximal value to sequences where all its subsequences occur at equal probability and a value of 0 or close to 0 to sequences consisting of periodic repetitions of short subsequences. The practical determination of the (higher order) Shannon entropy however is limited by the finite lengths of sequences which often does not permit a precise estimation of the probability distribution of their constitutive symbols.

Further possible measures are for example the Lempel-Ziv complexity (Lempel, L B and Ziv, J (1976) On the complexity of finite sequences. IEEE Trans. Inf. Theory IT-22, 75-81), the grammar complexity (Ebeling, W; Jimenez-Montano, M A (1980) On Grammars, Complexity and Information Measures of Biologoical Macromolecules. Mathematical Bioscience 52, 53-71), the algorithmic complexity (Chaitin, 1990) and the conditial entropy.

Secondly, said primer molecules are also characterized in that every possible combination of any two primer molecules, in the set, has a melting temperature below a specified threshold temperature. That way the accumulation of dimers caused by the binding of two primer molecules to each other in said reaction mixture is excluded. The number of primer pairs used in that step can be any between one and n, leading to one or n amplificates respectively (n being a natural number).

As mentioned in the text the word “dimer” refers to a secondary structure formed by the hybridization of two primer molecules to each other.

As referred to in the text ‘melting temperature’ refers to the temperature at which 50% of the nucleic acid molecules are in duplex and 50% are denatured under standard reaction solution conditions.

Some primer design tools disqualify a primer if, besides the target sequence, a second identical sequence can be found in the template. However, due to the higher probability of a bisulfite primer to mismatch with non-identical bisulfite treated DNA, it is an embodiment of this invention that only those primers are allowed to be used in said amplification method, for which no sequence homology can be found, to the extent that even those sequences that are different and/or mismatching in several nucleotides are excluded. However, this would exclude primer molecules unnecessarily. Therefore they are only excluded if two primer molecules match to the template in a distance allowing for the amplification of an unwanted product. This test is performed by means as, for example, the Electronic PCR. Electronic PCR (e-PCR) is an in silico virtual PCR carried out in order to assess the suitability of primer molecules prior to in vitro PCR. In the scope of this invention this testing will be called ‘virtual testing’ and it will be referred to as “virtually tested” or “virtually testing”.

Thirdly, the primers used in step 3 of this invention are characterized, in that every possible combination of two primer molecules, in said reaction mixture, does not lead to the amplification of an additional unwanted product, when virtually testing for amplification using the treated and the untreated nucleic acid sample as template, even under conditions allowing for at least one base but not more than 20% of the total number of bases per sequence mismatching per primer. In the scope of this invention it is to be understood that those primer molecules are considered to bind to the template for which a template sequence exists that is in at least 80% of its nucleotide sequence identical to the target sequence the primer originally has been designed for. For example, a primer molecule of 50 nucleotides length is considered to still hybridize to a template sequence that differs in less than 11 nucleotides (=is identical in at least 80% of its nucleotide sequence) from the according target sequence. If a match is considered to be possible it has to be tested whether this match would lead to the amplification of an unwanted product. This can be done with the use of a program similar to e-PCR (see below).

Especially preferred is an embodiment of said method wherein the ability of said primer molecules to amplify an unwanted product is tested by means of in silico PCR, taking as template nucleic acid the coding strand of the treated sample, the non-coding strand of the treated sample and both of the strands of the untreated sample. It is especially preferred to perform the virtual testing with a tool like electronic PCR on the pretreated, preferably bisulfite treated, template sequence consisting of the treated sense and the treated anti-sense strand, and, on the unconverted template.

Furthermore it is preferred that this treatment is bisulfite treatment and hence the nucleic acid template is the bisulfite converted coding strand of the human genome, the bisulfite converted non-coding strand of the human genome and both of the strands of the untreated human genome. Preferred is an embodiment of said method wherein the ability of said primer molecules to amplify an unwanted product is tested by means of electronic PCR, hereby taking as template nucleic acid the bisulfite converted coding strand of the human genome, the bisulfite converted non-coding strand of the human genome and both of the strands of the untreated human genome.

It is preferred that the number of mismatches allowed for when virtually testing the amplification of unwanted products according to step 3 c) of the invention is less than 20% of the number of nucleotides of the primer.

It is also preferred that the number of mismatches allowed or when virtually testing the amplification of unwanted products according to step 3 c) of the invention is less than 10% of the number of nucleotides of the primer.

It is especially preferred that the number of mismatches allowed for when virtually testing the amplification of unwanted products according to step 3 c) of the invention is less than 5% of the number of nucleotides of the primer.

It is a preferred embodiment of this invention that the number of mismatches allowed for when virtually testing the amplification of unwanted products according to step 3 c) of the invention is less than seven.

It is especially preferred that the number of mismatches allowed for when virtually testing the amplification of unwanted products according to step 3 c) of the invention is less than five.

It is another preferred embodiment of this invention that the number of mismatches allowed for when virtually testing the amplification of unwanted products according to step 3 c) of the invention is less than three.

It is especially preferred in the scope of this invention that the number of mismatches allowed for when virtually testing the amplification of unwanted products according to step 3 c) of the invention is one.

It is also included in the scope of this invention to consider such primer molecules as being sufficiently similar to facilitate their binding to the template sequence, for which a template sequence can be found that differs in the number of nucleotides but is otherwise identical to the target sequence. When the alignment of the primer and the template sequence leads to a gap of up to 20% of the nucleotides of one sequence, preferably of the primer sequence, this shall still be considered to be sufficient for binding and hence potentially leading to the amplification of an unwanted product. Therefore these primers also need to be tested with the means of virtual PCR (for example with a program like e-PCR). Only if this test reveals the virtual amplification of an unwanted product caused by the combination of two primers, the according primer pairs are excluded from the set of selected pairs.

It is preferred that the number of nucleotides creating one gap, in one of the sequences, when aligning the primer molecule sequence with the template sequence, allowed for when virtually testing the amplification of unwanted products according to step 3 c) of the invention is less than 20% of the number of nucleotides of the primer molecule.

It is also preferred that the number of nucleotides creating one gap, in one of the sequences, when aligning the primer molecule sequence with the template sequence, allowed for when virtually testing the amplification of unwanted products according to step 3 c) of the invention is less than 10% of the number of nucleotides of the primer molecule.

It is preferred that the number of nucleotides creating one gap, in one of the sequences, when aligning the primer molecule sequence with the template sequence, allowed for when virtually testing the amplification of unwanted products according to step 3 c) of the invention is less than 5% of the number of nucleotides of the primer molecule.

Both of these situations, mismatching due to an alternative nucleotide or no-matching due to a missing nucleotide, are meant to be covered in the expression describing those primer molecules that will eventually be selected: “said primer molecules are characterized in that every combination of two primer molecules, under conditions allowing for one or more base mismatches per primer, does not lead to the amplification of an unwanted product when virtually tested using the treated and the untreated sample nucleic acids as template”.

It is also preferred that the primer molecules that exceed a pre-specified melting temperature when binding to the template have to be virtually tested for amplification of unwanted products using the treated and the untreated sample nucleic acids as template according to step 3 c) of the method.

The basic problem of finding a primer specific enough to give only one product on the little complex bisulfite DNA, is finally solved by testing each potential primer pair for hybridization across the whole bisulfite converted human genome. This requires translating the whole human genome sequence information virtually into its bisulfite treated version before performing a similarity search against the primer pairs, which is based on a method like the so called e-PCR (Schuler G. D. (1997) Sequence Mapping by electronic PCR. Genome Research 7(5): 541-550). However, as the bisulfite conversion results in two no longer complementary strands this virtual hybridization test needs to be done against both bisulfite converted strands. In addition in most cases the template DNA is contaminated with unconverted genomic DNA. To also exclude unwanted amplification on the unconverted DNA as template, the same hybridization test has to be performed a third time using the whole human genome sequence as a template.

Therefore it is a preferred embodiment of this invention that the ability of said primer molecules to amplify an unwanted product is tested by means such as electronic PCR.

In the last step of the method said amplified target nucleic acid gets detected by any means standard to one skilled in the art.

In a preferred embodiment of this method the set of primer molecules is comprised of at least two primer molecules but not more than 64 primer molecules, given the number is a multiple of 2; in other words, the set is comprised of 1-32 primer pairs.

In another preferred embodiment of this method the set of primer molecules is comprised of between 2 and 32 primer molecules, given the number is a multiple of 2; in other words the set is comprised of 1-16 primer pairs.

In a preferred embodiment of this method, said primer molecule comprises at least one nucleotide within the last three nucleotides from the 3′ end of the molecule, wherein said nucleotide is complementary to a nucleotide of the target sequence that, as a result of the treatment performed in step 2) of the invention, changed its hybridization behavior.

It is a preferred embodiment of this method, that said primer molecule comprises at least one nucleotide within the last three nucleotides from its 3′ end that is complementary to a nucleotide of the target sequence that was converted by the treatment performed in step 2 of the method to another base exhibiting an alternative base pairing behavior.

In an especially preferred embodiment said nucleotide is a cytosine prior to the treatment that converts unmethylated cytosines. In a preferred embodiment said treatment is bisulfite treatment. Said primer molecule comprises at least one nucleotide within the last three nucleotides from the 3′ end of the molecule, wherein said nucleotide is complementary to a cytosine, that was converted by bisulfite treatment to another base exhibiting the base pairing behavior of thymine.

This is to exclude binding of said primer molecules to the remaining untreated or un-sufficiently treated nucleic acids, which might still serve as template nucleic acid in the PCR.

Furthermore it is a preferred embodiment of this invention that said primer molecules do not form loops or hairpins on their own or with each other.

In another preferred embodiment of the method said primer molecules do not form dimers with each other.

In the text the word ‘hairpin’ is taken to mean a secondary structure formed by a primer molecule when the 3′ terminal region of said nucleic acid hybridizes to the 5′ terminal region of said nucleic acid forming a double stranded stem structure and wherein only the central region of the primer is single stranded.

As described in the text the word ‘loop’ refers to a secondary structure formed by a primer molecule when two or more nucleotides of said molecule hybridize thereby forming a secondary structure comprising a double stranded structure one or more base pairs in length and further comprising a single stranded region between said double stranded region.

The binding of a primer molecules 3 end to any part of a second primer molecule in the set needs to be avoided. Otherwise the polymerase would extend the first primer using the second primer as template, which would lead to a new unwanted product, an extended primer, or rather a primer-hybrid, which would serve as the preferred template for the next round of the polymerase chain reaction and thereby prevent a sufficient amplification of the wanted product.

Therefore it is another preferred embodiment of this method that each of said primer molecules is characterized in that the last at least 5 bases at the 3′ end of said primer molecule are not complementary to the sequence of any other primer molecule in the set.

It is also preferred that said primer molecules do not bind to nucleic acids which prior to treatment of step 2 contained a 5′-CG-3′ site. This would lead to a binding of the primers to bisulfite treated nucleic acids, specifically depending on their cytosines methylation status. A CG corresponding primer would bind to the treated methylated version only, whereas a primer corresponding to TG would bind to the treated unmethylated version of these nucleic acids only. It is therefore preferred that said primer molecules do not contain nucleic acid sequences complementary or identical to nucleic acid sequences which prior to treatment of step 2 contained a 5′-CG-3′ site.

In a preferred embodiment of this method said primer molecules are of a specified size range.

It is especially preferred that these primers are comprised of 16-50 nucleotides.

In a preferred embodiment of this method said primer molecules do not comprise sequences that are complementary to regions of the target nucleic acids that contained specified restriction enzyme recognition sites prior to the treatment that altered the unmethylated cytosines base pairing behavior. It is preferred that said primers are complementary to target sequences which prior to the treatment performed in step 2 of the invention did not contain specified restriction enzyme recognition sites.

By selecting for the right primer molecules also the amplificates sequence is determined. That is why it has to be taken into account to only use those primer molecules that lead to amplification of nucleic acids containing a reasonable high number of CpG sites to be analyzed. Due to the treatment of step 2 of this invention these CpG sites, depending on the methylation status of the cytosine, are converted and will therefore either appear as CG dinucleotides or as TG dinucleotides in the amplificate.

It is preferred that said primer molecules amplify regions of nucleic acids that prior to bisulfite treatment comprise of more than eight 5′-CG-3′ sites also referred to as CG dinucleotides.

It is also preferred that said primer molecules amplify regions of nucleic acids that prior to bisulfite treatment comprise of more than six 5′-CG-3′ sites also referred to as CG dinucleotides.

It is also preferred that said primer molecules amplify regions of nucleic acids that prior to bisulfite treatment comprise of more than four 5′-CG-3′ sites also referred to as CG dinucleotides and finally it is especially preferred that said primer molecules amplify regions of nucleic acids that prior to bisulfite treatment comprise of more than two 5′-CG-3′ sites also referred to as CG dinucleotides.

Said primer molecules lead to amplificates within a specified size range.

It is a preferred embodiment of this sequence that said primer molecules lead to amplificates which are comprised of at least 50 bp but not more than 2000 bp.

Especially preferred are primer molecules that lead to amplificates which are comprised of at least 80 bp but not more than 1000 bp.

Furthermore a method is preferred wherein said primer molecules lead to amplificates of treated nucleic acids which prior to the treatment which altered the unmethylated cytosines base pairing behavior did not contain restriction enzyme recognition sites. Said primer molecules lead to amplificates that are amplified regions of the treated nucleic acids which prior to the treatment performed in step 2) of the method did not contain specified restriction enzyme recognition sites.

A further subject of this invention is a method on how to produce said primer molecules. The main step of producing a primer molecule is determining its sequence. In the following the phrase “primer design” will be used instead of primer production, whenever it is referred to the step of determining said specific primer sequences. Designing primer molecules is a process which as such is well known to scientists skilled in the art. The programs usually used for this purpose are such as PRIMER3 or OSP (Rozen S and Skaletsky H (2000) PRIMER3 on the WWW for general users and for biologist programmers. Methods Mol Biol 132: 365-386; Hillier L and Green P (1991) OSP: A computer program for choosing PCR and DNA sequencing primers. PCR Methods and Applications 1: 124-128). Other primer design systems (like described in EP-A 1136932) are often based on those commonly known programs.

An embodiment of this invention takes advantage of using a program like PRIMER3 first, to then add a number of steps that finally result in an advanced method of designing primers that are specifically useful for amplifying sequences of low complexity.

In the first step of this method for designing specific primer molecules for nucleic acids of low complexity, primer pairs that amplify single products are selected by applying standard tools of primer design known in the art, like for example the program PRIMER3 (Rozen, S and Skaletsky, H (2000) Methods Mol Biol 132: 365-386).

In the second step of the method said primer pairs are tested whether or not one of its primer molecules when hybridizing to any other primer molecule in the set exceeds a specified threshold melting temperature TM. If this is the case the primer pair that comprises of said primer is excluded from the set of potentially combined pairs.

In the third step of the method the number of previously selected primer pairs, is reduced to a smaller number by implementing as new criteria a measure for the primer sequence's complexity. Primer pairs that consist of a primer molecule which does not meet said criteria are excluded.

The basic problem of finding a primer specific enough to give only one product on the little complex bisulfite DNA, is finally solved by testing each potential primer pair for hybridization across the whole bisulfite converted human genome. This requires translating the whole human genome sequence information virtually (as in “in silico”) into its treated, for example bisulfite treated, version before performing a similarity search against the primer pairs, which is based on a method like the so called e-PCR (Schuler G. D. (1997) Sequence Mapping by electronic PCR. Genome Research 7(5): 541-550). However, as the bisulfite conversion results in two different versions of the double helix whose sense and anti-sense strands are no longer mutually complementary, this in silico amplification needs to be performed on both bisulfite converted versions of the genome. In addition in most cases the template DNA is contaminated with unconverted genomic DNA. It cannot be excluded that single cytosines or longer runs of DNA remain unconverted or are only converted incompletely by the bisulfite treatment. To also exclude unwanted amplification of the unconverted DNA as template, the same hybridization test has to be performed a third time using the whole human genome sequence as a template.

As this is quite some effort and requires time (CPU time) this is the fourth and last step of this design method, that is absolved prior to the final testing in a “wet”, lab based, experiment.

In addition to improve the specificity of said primer molecules the stringency of the selection criteria is increased: Some standard primer design tools disqualify a primer if in the template sequence, a second identical sequence, besides the target sequence, can be found. That way mispriming at rather stringent hybridization conditions is avoided. This mispriming would not necessarily lead to an additional unwanted product, but would lead to the dilution of the primer molecules available for amplification. This selection has been performed in step one already (for example by PRIMER3). However, due to the higher probability of a bisulfite primer molecule to mismatch with non-identical bisulfite treated DNA, there is still a chance for said primer molecules to misprime even when up to 20% of the nucleotides of the primer sequence differ. Therefore it is claimed in this invention to only use primer molecules for which not even a weak sequence homology can be found. However, this would exclude primer molecules unnecessarily. Therefore they are only excluded if two primer molecules match to the template and amplify an unwanted product. This test is performed by means as, for example, the Electronic PCR. Electronic PCR (e-PCR) is an in silico virtual PCR carried out in order to asses the suitability of primers prior to in vitro PCR.

In the fourth step of the method on how to design these primers it is therefore tested whether there are any regions of the template nucleic acid, said template being comprised of the sense and the anti-sense strand of the treated and the untreated nucleic acids, that are identical in sequence with the primer molecule to more than 80% and if those primer molecules are able to amplify an unwanted product. If this is the case, the primer pair comprising said primer molecule is excluded from the selection.

The template nucleic acid is comprised of the treated template nucleic acid and the untreated template nucleic acid. The treated nucleic acid in itself is comprised of a two strands which after treatment are not complementary to each other anymore. This virtual testing for example can be performed as described by Gregory Schuler in his article (cited above) about sequence mapping by “Electronic PCR”. The primer pairs remaining can be used to specifically amplify regions of nucleic acids of low complexity, which is the aim of this invention. Hence step 4 of the design method is the virtual testing of each possible primer pair combination, under pre-specified conditions at a stringency allowing for one or more base pair mismatches, as to whether no unwanted nucleic acids are amplified. Said virtual testing is carried out upon both untreated and treated nucleic acids. The wording “possible combinations” refers to all combinations that are possible within a set of primer pairs to be used in one amplification reaction vessel.

In a preferred embodiment an additional step is added following the virtual testing, which is testing in a lab based single PCR assay all those pairs that remained, whether the desired amplificate can be obtained or not. If that is the case, the chosen pairs can be used to specifically amplify those regions of nucleic acids of low complexity according to the method as described before.

In a specially preferred embodiment the first step of the design method is characterized as selecting a pool of possible primer pairs per amplificate by means of a standard PCR primer design program using said nucleic acids as template that have been masked for repeats and SNPs considering the following factors: length of amplificate, length of primer, melting temperature of the primer molecule, dimer formation parameters, loop formation parameters, exclusion of unidentified or ambiguous nucleotides in the primer sequence, exclusion of restriction enzyme recognition sites.

In a preferred embodiment of this invention this measure of complexity is a measure of linguistic complexity as defined by Bolshoy et al. (see above). Those primer pairs are excluded from the previously selected ones, which comprise of one primer that doesn't reach a set level of this linguistic complexity.

In another preferred embodiment of this invention this measure of complexity is a measure of Shannon entropy (as described before).

In an especially preferred embodiment of this design method, prior to performing step d) the additional step of excluding primer pairs from the remaining primer pairs which consist of a primer molecule that comprises of at least one CpG site, is carried out.

In an especially preferred embodiment of this method according to the design of said primers, prior to performing step d) the additional step of excluding primer pairs from the remaining pairs when one of its primer molecules does not contain at least one nucleotide within the last three nucleotides from the 3′ end of the molecule wherein said nucleotide is complementary to a nucleotide of the target sequence that was converted to a different nucleotide by bisulfite treatment, is carried out.

In an especially preferred embodiment of this method according to the production of said primers, prior to performing step d) the additional step, of excluding primer pairs from the remaining primer pairs which amplify a nucleic acid that did not prior to treatment with bisulfite contain a minimum of two CpG sites, is carried out.

In an especially preferred embodiment of this method according to the production of said primers, prior to performing step d) the additional step of excluding primer pairs from the remaining primer pairs when one of its primer molecules contains more than 5 bases at its 3′ end that are complementary to any other primer molecules sequence in the set, is carried out.

In an especially preferred embodiment of this method according to the production of said primers, prior to performing step d) the additional step of excluding from the remaining primer pairs those pairs, which comprise of one primer molecule that in combination with another primer molecule in the set amplifies an unwanted product, when virtually testing according to step 3 c) of the amplification method under conditions allowing for a number of mismatching nucleotides of 20% of the number of nucleotides of the primer molecule, is carried out.

In an especially preferred embodiment of this method according to the production of said primers, prior to performing step d) the additional step of excluding from the remaining primer pairs those pairs, which comprise of one primer molecule that in combination with another primer molecule in the set amplifies an unwanted product, when virtually testing according to step 3 c) of the amplification method under conditions allowing for a number of nucleotides creating one gap, when aligning the primer molecule sequence with the template sequence, of up to 20% of the number of nucleotides of the primer molecule, is carried out.

In an especially preferred embodiment of this method according to the production of said primers, prior to performing step d) the additional step of excluding from the remaining primer pairs those pairs, which comprise of one primer molecule that in combination with another primer molecule in the set amplifies an unwanted product, when virtually testing according to step 3 c) of the amplification method under conditions allowing for four or less mismatching base pairs, is carried out.

In an especially preferred embodiment of this method according to the production of said primers, prior to performing step d) the additional step of excluding from the remaining primer pairs those pairs, which comprise of one primer molecule that in combination with another primer molecule in the set amplifies an unwanted product, when virtually testing according to step 3 c) of the amplification method under conditions allowing for two or less mismatching base pairs, is carried out.

The following example is intended to illustrate the invention:

### EXAMPLE

Here we present experimental data that shows that multiplex PCRs designed with a tool according to this invention are more successful compared to multiplex PCRs not designed in this manner.

It is the aim of the experiment to amplify 40 different nucleic acids. The genomic regions of interest are given in the sequence protocol (SEQ ID 41-80). These genomic sequences were translated into their bisulfite converted versions and served as templates for amplification of specific regions with the primer sequences described as follows.

Primer molecule pairs used for single PCRs were originally designed with the use of the standard primer design program PRIMER3 (as mentioned in the description). The criteria used in that step will not be discussed in detail. This selection however provides several possible primer pairs per amplificate. Following the present invention these primer pairs were selected further, according to the following criteria:
The restriction enzyme recognition site to be excluded from the genomic nucleic acid (which subsequent to bisulfite conversion becomes the template for the PCR amplification step) is: GTTTAAAC.
The minimum length of the primer molecule is 18 nucleotides. The maximum length is 27 nucleotides.

Ideally the primer consists of 22 nucleotides.
The minimum required measure of linguistic complexity is 0.2.
The minimum melting temperature of a primer molecule is 54° C. and the maximum melting temperature is 57° C. The ideal melting temperature however is 55° C.
The minimum length of an amplificate is 100 bp and the maximum length is 500 bp.
The minimum number of CpG sites, that were present in the region of the nucleic acid, prior to bisulfite treatment, that was amplified is 4.
The number of mismatch bases allowed for when virtually testing the primer pairs according to the invention for amplification of an unwanted product with the help of e-PCR (Electronic PCR) is 2.

The use of this invention, that is the use of either the design method, being the subject of the invention, and/or performing the steps of said method as described above (assuming a set size of 1) leads to the selection of the following 40 optimized primer molecule pairs:

TABLE 1

| | | | | |
|---|---|---|---|---|
| | | | number | starting position of |
| | | | indicating | primer in the bisul- |
| | amplificate | SEQ | primer | fite converted se- |
| primer sequence | identifier | ID | direction | quence of the ROI |

| | | | | |
|---|---|---|---|---|
| AATCCTCCAAATTCTAAAAACA | 2025 | 81 | 0 | 1816 |
| AGGAAAGGGAGTGAGAAAAT | 2025 | 82 | 1 | 2138 |
| GGATAGGAGTTGGGATTAAGAT | 2044 | 83 | 0 | 2070 |
| AAATCTTTTTCAACACCAAAAT | 2044 | 84 | 1 | 2483 |
| AACCCTTTCTTCAAATTACAAA | 2045 | 85 | 0 | 1340 |
| TGATTGGGTTTTAGGGAAATA | 2045 | 86 | 1 | 1687 |
| TTGAAAATAAGAAAGGTTGAGG | 2106 | 87 | 0 | 1481 |
| CTTCTACCCCAAATCCCTA | 2106 | 88 | 1 | 1764 |
| TGTTTGGGATTGGGTAGG | 2166 | 89 | 0 | 2226 |
| CATAACCTTTACCTATCTCCTCA | 2166 | 90 | 1 | 2437 |
| TTTTAGATTGAGGTTTTAGGGT | 2188 | 91 | 0 | 101 |
| ATCCATTCTACCTCCTTTTTCT | 2188 | 92 | 1 | 598 |
| GGAGGGGAGAGGGTTATG | 2191 | 93 | 0 | 133 |
| TACTATACACACCCCAAAACAA | 2191 | 94 | 1 | 506 |
| TTTTGGGAATGGGTTGTAT | 2194 | 95 | 0 | 1628 |
| CTACCCTTAACCTCCATCCTA | 2194 | 96 | 1 | 1996 |
| TTGTTGGGAGTTTTTAAGTTTT | 2212 | 97 | 0 | 1711 |
| CAAATTCTCCTTCCAAATAAAT | 2212 | 98 | 1 | 2063 |
| GTAATTTGAAGAAAGTTGAGGG | 2267 | 99 | 0 | 1709 |
| CCAACAACTAAACAAAACCTCT | 2267 | 100 | 1 | 2004 |
| GGAGTTGTATTGTTGGGAGA | 2317 | 101 | 0 | 1110 |
| TAAAACCCCAATTTTCACTAA | 2317 | 102 | 1 | 1388 |
| TTTGTATTAGGTTGGAAGTGGT | 2383 | 103 | 0 | 1 |
| CCCAAATAAATCAACAACAACA | 2383 | 104 | 1 | 285 |
| GATTTTTGGAGAGGAAGTTAAG | 2387 | 105 | 0 | 789 |
| AAAACTAAAAACCAAACCCATA | 2387 | 106 | 1 | 1169 |
| TGGGGTTAGTTTAGGATAGG | 2391 | 107 | 0 | 1353 |
| CTTAAAAACACTAAAACTTCTCAAA | 2391 | 108 | 1 | 1750 |
| TTTTTGTATTGGGGTAGGTTT | 2395 | 109 | 0 | 547 |
| CCCAACTATCTCTCTCCTCTATAA | 2395 | 110 | 1 | 1094 |
| ATTAGAAGTGAAAGTAATGGAATTT | 2401 | 111 | 0 | 381 |
| TCAATTTCCAAAAACCAAC | 2401 | 112 | 1 | 795 |
| GGGATGGGTTATTAGTTGTAAA | 2453 | 113 | 0 | 1867 |
| CCTTCACACAAAACTACAAAAA | 2453 | 114 | 1 | 2139 |
| TAATTGAAGGGGTTAATAGTGG | 2484 | 115 | 0 | 1861 |
| AAAACCAAAACCAAAACTAAAA | 2484 | 116 | 1 | 2252 |
| AGTGGATTTGGAGTTTAGATGT | 2512 | 117 | 0 | 1016 |
| AACAAAATAAAAACTTCTCCCA | 2512 | 118 | 1 | 1446 |
| TAGGGGAAAAGTTAGAGTTGAG | 2741 | 119 | 0 | 1413 |
| CCCATTAACCCACAAAAA | 2741 | 120 | 1 | 1888 |
| ATTTTAGTTTGTGAAATGGGAT | 2745 | 121 | 0 | 1685 |
| TCTTAACCAATAACCCCTCAC | 2745 | 122 | 1 | 2097 |
| GTGGGTTTTGGGTAGTTATAGA | 2746 | 123 | 0 | 1679 |
| TAACCTCCTCTCCTTACCAA | 2746 | 124 | 1 | 2163 |
| TAGGATGGGGAGAGTAATGTTT | 2747 | 125 | 0 | 972 |
| ACAACTTATCCAACTTCCATTC | 2747 | 126 | 1 | 1448 |
| TCCCACAAAAACTAAACAATTA | 2749 | 127 | 0 | 1370 |
| AGGTTTTAGATGAAGGGGTTT | 2749 | 128 | 1 | 1789 |
| TTTGGAGGGTTTAGTAGAAGTTA | 2751 | 129 | 0 | 88 |
| CCCAATAATCACAAAATAAACA | 2751 | 130 | 1 | 567 |
| ATACAACCTCAAATCCTATCCA | 2752 | 131 | 0 | 228 |
| AGGGAGAAGGAAGTTATTTGTT | 2752 | 132 | 1 | 712 |
| GGAAGATGAGGAAGTTGATTAG | 2755 | 133 | 0 | 1000 |
| CCTACAACCCTATCCTCTAAAA | 2755 | 134 | 1 | 1371 |
| TTAGTAGGGGTGTGAGTGTTTT | 2831 | 135 | 0 | 1313 |
| CAAACAAAACTTCTATCTCAACC | 2831 | 136 | 1 | 1499 |
| TTATAGGGTTGAGTTTGGGAT | 2850 | 137 | 0 | 2100 |
| TAAACAAACAACAAATCTTCCA | 2850 | 138 | 1 | 2400 |
| TGAAAATGAAGGTATGGAGTTT | 2852 | 139 | 0 | 1262 |
| TTAAAACCATATAATCCCTCCA | 2852 | 140 | 1 | 1583 |
| TATGTTTGGTTTTGTTTTGAGA | 2859 | 141 | 0 | 1093 |
| AACCCCATCACTTTTATTTCTT | 2859 | 142 | 1 | 1491 |
| GGGTGTAGAAGTGTTTAGGTTT | 2861 | 143 | 0 | 2385 |
| TTTCTCCCCTTACAACAATAAC | 2861 | 144 | 1 | 2732 |
| TCCCCTTCCAACTATATCTCTC | 2864 | 145 | 0 | 884 |
| TGAGAGTGTTTTAGGGAAGTTT | 2864 | 146 | 1 | 1175 |
| AAAACCAAAACATAAACCAAAA | 2867 | 147 | 0 | 1312 |
| GATTAGGAGGGTTTGTTGAGAT | 2867 | 148 | 1 | 1701 |
| AATGGTTGATGATTTTGGTTT | 2961 | 149 | 0 | 2039 |
| ACTCTCTTCCCTATACCCCTAA | 2961 | 150 | 1 | 2311 |
| AGTTAGAAGAGGAGTTAGGATGG | 3511 | 151 | 0 | 1340 |
| TAATTTTCCAATACCCATTTTC | 3511 | 152 | 1 | 1711 |
| TGTTAGTAGAGTTTTAGGGAGGTT | 3532 | 153 | 0 | 1135 |
| ACACTACCTATCCTTACCCCAC | 3532 | 154 | 1 | 1592 |
| TTTTTGTTTTTATGGGGTGTAT | 3534 | 155 | 0 | 1909 |
| TTAAATATCCCTTCCTTAACCA | 3534 | 156 | 1 | 2385 |
| TGGGTAGTATTTTTGTTGGTTT | 3538 | 157 | 0 | 956 |
| CCTAAAAACTCTCTCATCCTCA | 3538 | 158 | 1 | 1414 |
| AGTGGTTTAGGAGTATTTGGTTA | 3540 | 159 | 0 | 659 |
| AACTCCCTCCATCTACAATATC | 3540 | 160 | 1 | 1064 |

These primer pairs lead to the amplification of specific regions (amplificates Seq IDs 1-40) of the bisulfite converted sequences of the genomic ROIs (Seq IDs 41-80) of interest. The ROIs can be identified by the four digit number that specifies the ROI and the corresponding amplificate—as indicated in the following table.

TABLE 2

| SEQ ID | Class | Identifier | Kind of DNA |
|---|---|---|---|
| 1 | amplificate | 2025 | bisulfite |
| | | | sequence |
| 2 | amplificate | 2044 | bisulfite |
| | | | sequence |
| 3 | amplificate | 2045 | bisulfite |
| | | | sequence |
| 4 | amplificate | 2106 | bisulfite |
| | | | sequence |
| 5 | amplificate | 2166 | bisulfite |
| | | | sequence |
| 6 | amplificate | 2188 | bisulfite |
| | | | sequence |
| 7 | amplificate | 2191 | bisulfite |
| | | | sequence |
| 8 | amplificate | 2194 | bisulfite |
| | | | sequence |
| 9 | amplificate | 2212 | bisulfite |
| | | | sequence |
| 10 | amplificate | 2267 | bisulfite |
| | | | sequence |
| 11 | amplificate | 2317 | bisulfite |
| | | | sequence |
| 12 | amplificate | 2383 | bisulfite |
| | | | sequence |
| 13 | amplificate | 2387 | bisulfite |
| | | | sequence |
| 14 | amplificate | 2391 | bisulfite |
| | | | sequence |
| 15 | amplificate | 2395 | bisulfite |
| | | | sequence |
| 16 | amplificate | 2401 | bisulfite |
| | | | sequence |
| 17 | amplificate | 2453 | bisulfite |
| | | | sequence |
| 18 | amplificate | 2484 | bisulfite |
| | | | sequence |
| 19 | amplificate | 2512 | bisulfite |
| | | | sequence |
| 20 | amplificate | 2741 | bisulfite |
| | | | sequence |
| 21 | amplificate | 2745 | bisulfite |
| | | | sequence |
| 22 | amplificate | 2746 | bisulfite |
| | | | sequence |
| 23 | amplificate | 2747 | bisulfite |
| | | | sequence |
| 24 | amplificate | 2749 | bisulfite |
| | | | sequence |
| 25 | amplificate | 2751 | bisulfite |
| | | | sequence |
| 26 | amplificate | 2752 | bisulfite |
| | | | sequence |
| 27 | amplificate | 2755 | bisulfite |
| | | | sequence |
| 28 | amplificate | 2831 | bisulfite |
| | | | sequence |
| 29 | amplificate | 2850 | bisulfite |
| | | | sequence |
| 30 | amplificate | 2852 | bisulfite |
| | | | sequence |
| 31 | amplificate | 2859 | bisulfite |
| | | | sequence |
| 32 | amplificate | 2861 | bisulfite |
| | | | sequence |
| 33 | amplificate | 2864 | bisulfite |
| | | | sequence |
| 34 | amplificate | 2867 | bisulfite |
| | | | sequence |
| 35 | amplificate | 2961 | bisulfite |
| | | | sequence |
| 36 | amplificate | 3511 | bisulfite |
| | | | sequence |
| 37 | amplificate | 3532 | bisulfite |
| | | | sequence |
| 38 | amplificate | 3534 | bisulfite |
| | | | sequence |
| 39 | amplificate | 3538 | bisulfite |
| | | | sequence |
| 40 | amplificate | 3540 | bisulfite |
| | | | sequence |
| 41 | ROI | 2025 | genomic |
| | | | sequence |
| 42 | ROI | 2044 | genomic |
| | | | sequence |
| 43 | ROI | 2045 | genomic |
| | | | sequence |
| 44 | ROI | 2106 | genomic |
| | | | sequence |
| 45 | ROI | 2166 | genomic |
| | | | sequence |
| 46 | ROI | 2188 | genomic |
| | | | sequence |
| 47 | ROI | 2191 | genomic |
| | | | sequence |
| 48 | ROI | 2194 | genomic |
| | | | sequence |
| 49 | ROI | 2212 | genomic |
| | | | sequence |
| 50 | ROI | 2267 | genomic |
| | | | sequence |
| 51 | ROI | 2317 | genomic |
| | | | sequence |
| 52 | ROI | 2383 | genomic |
| | | | sequence |
| 53 | ROI | 2387 | genomic |
| | | | sequence |
| 54 | ROI | 2391 | genomic |
| | | | sequence |
| 55 | ROI | 2395 | genomic |
| | | | sequence |
| 56 | ROI | 2401 | genomic |
| | | | sequence |
| 57 | ROI | 2453 | genomic |
| | | | sequence |
| 58 | ROI | 2484 | genomic |
| | | | sequence |
| 59 | ROI | 2512 | genomic |
| | | | sequence |
| 60 | ROI | 2741 | genomic |
| | | | sequence |
| 61 | ROI | 2745 | genomic |
| | | | sequence |
| 62 | ROI | 2746 | genomic |
| | | | sequence |
| 63 | ROI | 2747 | genomic |
| | | | sequence |
| 64 | ROI | 2749 | genomic |
| | | | sequence |
| 65 | ROI | 2751 | genomic |
| | | | sequence |
| 66 | ROI | 2752 | genomic |
| | | | sequence |
| 67 | ROI | 2755 | genomic |
| | | | sequence |
| 68 | ROI | 2831 | genomic |
| | | | sequence |
| 69 | ROI | 2850 | genomic |
| | | | sequence |
| 70 | ROI | 2852 | genomic |
| | | | sequence |
| 71 | ROI | 2859 | genomic |
| | | | sequence |
| 72 | ROI | 2861 | genomic |
| | | | sequence |
| 73 | ROI | 2864 | genomic |
| | | | sequence |
| 74 | ROI | 2867 | genomic |
| | | | sequence |
| 75 | ROI | 2961 | genomic |
| | | | sequence |
| 76 | ROI | 3511 | genomic |
| | | | sequence |
| 77 | ROI | 3532 | genomic |
| | | | sequence |
| 78 | ROI | 3534 | genomic |
| | | | sequence |
| 79 | ROI | 3538 | genomic |
| | | | sequence |
| 80 | ROI | 3540 | genomic |
| | | | sequence |

The second task in this example is to select from these 40 primer pairs those pairs which can be combined in five multiplex PCRs to amplify eight targets simultaneously.

The following steps, as disclosed in the invention, are performed for selection of those subsets:
The melting temperature of any combination of two of those primer molecules hybridizing to each other taking part in one multiplex experiment must be below 20° C.
The last seven nucleotides from the 3′ end of every primer molecule in a subset is used to check if those are complementary and/or binding to any other primer molecules' sequence used in the set.
The number of mismatch bases allowed for when virtually testing the primer pairs for amplification of an unwanted product is 2. For this step every possible combination of 16 primer molecules in one subset is checked for its ability to amplify an unwanted product. This is done by means of e-PCR (electronic PCR).

Having performed all these steps results in the selection of three different optimized sets of primer molecule pairs that can be used in multiplex PCRs. These sets are in the following described as a set of numbers. Each number refers to a specific amplificate and therefore also to a single primer pair (out of the list given above) which proved to be able to specifically amplify said nucleic acid in a single PCR experiment.

TABLE 3
optimized set 1

| | | | | | | | | |
|---|---|---|---|---|---|---|---|---|
| 8plex1 | 2194 | 2191 | 2391 | 2025 | 2961 | 3540 | 2861 | 2188 |
| 8plex2 | 2484 | 2106 | 2401 | 2850 | 3532 | 2044 | 2512 | 2852 |
| 8plex3 | 2453 | 2741 | 2867 | 2755 | 2267 | 2387 | 2864 | 2317 |
| 8plex4 | 2859 | 2383 | 2752 | 2747 | 2751 | 3511 | 2212 | 2746 |
| aplex5 | 3534 | 2395 | 2745 | 3538 | 2749 | 2166 | 2831 | 2045 |

optimized set 2

| | | | | | | | | |
|---|---|---|---|---|---|---|---|---|
| 8plex1 | 2166 | 2212 | 3511 | 2383 | 2745 | 2859 | 3534 | 2861 |
| 8plex2 | 2749 | 2191 | 2751 | 2395 | 2961 | 2512 | 2831 | 3538 |
| 8plex3 | 2850 | 2025 | 2188 | 2317 | 2391 | 2852 | 3540 | 2194 |
| 8plex4 | 2106 | 2387 | 2867 | 2864 | 2401 | 2747 | 2746 | 2453 |
| 8plex5 | 2044 | 2484 | 2267 | 2755 | 2752 | 2741 | 2045 | 3532 |

optimized set 3

| | | | | | | | | |
|---|---|---|---|---|---|---|---|---|
| 8plex1 | 2194 | 2391 | 2191 | 2749 | 2745 | 3538 | 2861 | 2961 |
| 8plex2 | 2166 | 2188 | 2859 | 2212 | 2864 | 2746 | 2383 | 2752 |
| 8plex3 | 2484 | 2401 | 2850 | 2852 | 2512 | 2755 | 2106 | 2044 |
| 8plex4 | 2867 | 2453 | 3532 | 2025 | 2741 | 2267 | 2317 | 2387 |
| 8plex5 | 3511 | 3534 | 2751 | 2747 | 2395 | 3540 | 2831 | 2045 |

Without the use of said invention, the selection would have been performed randomly and tested for successful application later. Three randomly chosen subsets are shown here.

TABLE 4
random set 1

| | | | | | | | | |
|---|---|---|---|---|---|---|---|---|
| 8plex1 | 2191 | 2194 | 2267 | 2741 | 3534 | 3511 | 2749 | 2747 |
| 8plex2 | 2391 | 2484 | 2867 | 2852 | 2453 | 2512 | 2025 | 3538 |
| 8plex3 | 2746 | 2212 | 2755 | 2045 | 2044 | 2188 | 2961 | 2864 |
| 8plex4 | 2831 | 2383 | 3540 | 2859 | 2861 | 2395 | 2401 | 2317 |
| 8plex5 | 2106 | 2751 | 2387 | 2745 | 2752 | 3532 | 2850 | 2166 |

random set 2

| | | | | | | | | |
|---|---|---|---|---|---|---|---|---|
| 8plex1 | 2045 | 2106 | 2212 | 2745 | 2044 | 2749 | 2752 | 2391 |
| 8plex2 | 2025 | 2831 | 2401 | 3540 | 2395 | 2484 | 2453 | 2961 |
| 8plex3 | 2194 | 2859 | 2746 | 2512 | 2267 | 2864 | 2861 | 2751 |
| 8plex4 | 2383 | 2166 | 2747 | 2387 | 3532 | 2741 | 2867 | 2852 |
| 8plex5 | 3534 | 2755 | 2850 | 2317 | 2191 | 3538 | 3511 | 2188 |

random set 3

| | | | | | | | | |
|---|---|---|---|---|---|---|---|---|
| 8plex1 | 2484 | 2850 | 2741 | 2747 | 2755 | 2745 | 2025 | 2746 |
| 8plex2 | 2383 | 3534 | 2861 | 2751 | 2749 | 2391 | 2188 | 2191 |
| 8plex3 | 2194 | 3538 | 2512 | 2961 | 2864 | 2867 | 2831 | 3532 |
| 8plex4 | 3511 | 2045 | 2387 | 2212 | 2166 | 2267 | 3540 | 2401 |
| 8plex5 | 2395 | 2317 | 2859 | 2453 | 2852 | 2106 | 2752 | 2044 |

The sequences of all of those amplificates and the according primers are given in the sequence protocol (primers SEQ IDs 81-160; amplificates SEQ IDs 1-40). SEQ IDs refer to the internal numbers used in these tables as is shown in TABLES 1 and 2.

To show if the use of the design method described herein was superior to the common method of selecting primers for simultaneous amplification randomly said multiplex PCRs were performed. This example hereby demonstrates the advantage of the method which is subject of the invention:

A total of 40 amplificates (with lengths ranging from 187-499 bp) were partitioned into five 8-plex PCRs using either of two strategies.

First: the grouping was based on the invention using said “optimised sets” (“designed group”).

Second: the grouping was done without using the selection criteria established by this invention using the “random sets” (“control group”).

Whether such grouping can improve the success rate of mPCRs was subsequently tested experimentally by comparing the number of true and false positives and false negatives for each of the two classes.

Each of the five mPCRs (multiplex PCRs) contained 8 primer pairs specific for 8 amplificates with one primer of each pair being labeled with a Cy-5 fluorescent tag. Only fragments that performed successfully in sPCR (singleplex PCR) using bisulfite-modified human DNA from whole blood were included in this study. Isomolar primer concentrations were used in a 20 μl PCR reaction volume and cycling was done for 42 cycles using a 96-well microtiter plate thermocycler.

Group assignments for the “optimized” and “random” groups were done in triplicate and all mPCRs were run at the same time such as to minimize experimental variation in PCR performance.

A mixture of the amplificates that were expected to be generated in a specific mPCR reaction but were generated in eight corresponding sPCR reactions was called sPCR-pool. Electrophoresis of sPCR-pool amplificates and mPCR amplificates was done simultaneously using the ALFexpress system (Amersham Pharmacia). In order to obtain the best comparability for mPCRs with their respective sPCR standard, these products were electrophoresed next to each other on the gels.

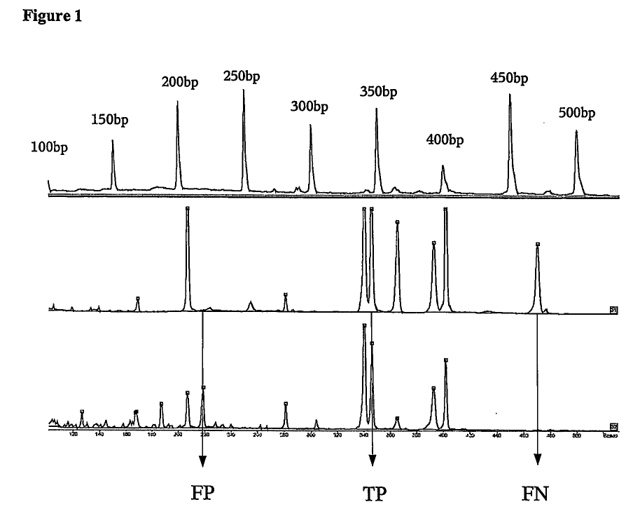

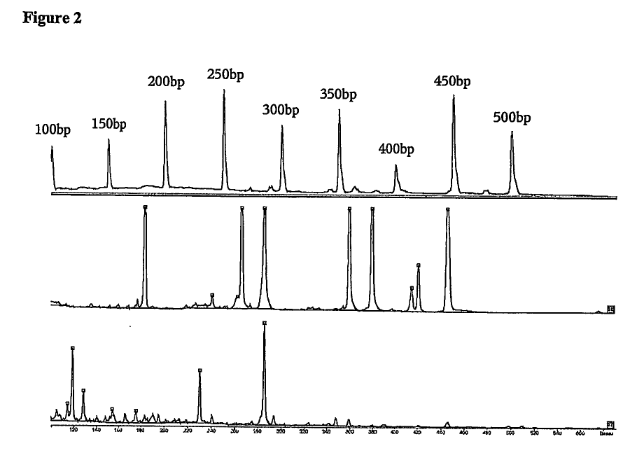

FIGS. 1 and 2 show examples of these results as electropherograms, given as ALFexpress output files.

Success or failure scoring for each mPCR was based on assessing the number of generated or absent fragments compared to their respective pool of sPCR fragments. Only fragments with peak areas equal or larger than 8% of the largest peak within one electropherogram were included into the analysis.

FIG. 1 illustrates a result of an 8-plex PCR based on a primer combination from the “optimized set”. The top graph in the figure shows peaks of size standards only. The second graph in the figure shows the electrophoresed mixture of the products from 8 singleplex PCRs. The third graph shows the products resulting from a multiplex PCR employing one of the optimized sets of primer combinations. By comparing these graphs it becomes visible that, in this specific example, there is only one false negative (FN) and three false positives (FP), whereas there are eight true positives (TP).

FIG. 2, however, illustrates a result of an 8-plex PCR based on a primer combination from the “control set”. The top graph in the figure shows peaks of size standards only. The second graph in the figure shows the electrophoresed mixture of the products from 8 singleplex PCRs. The third graph shows the products resulting from a multiplex PCR employing one of the randomly chosen sets, as is the state of the art. This graph clearly shows that, there are eight false negative and six false positive peaks, whereas there is only one true positive. Hence, for this specific example we have demonstrated the superiority of the design method.

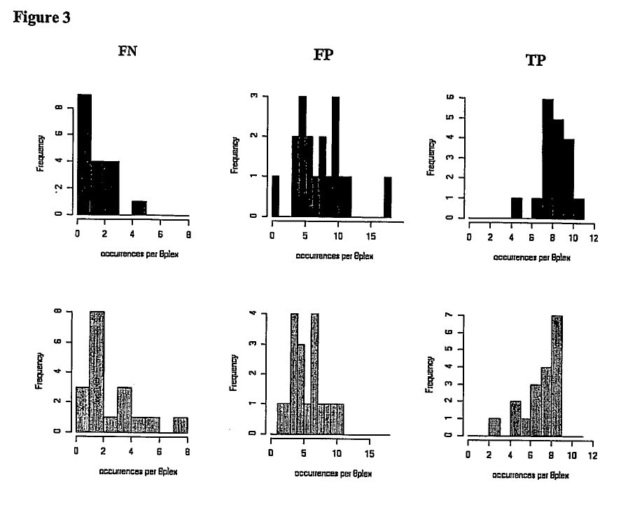

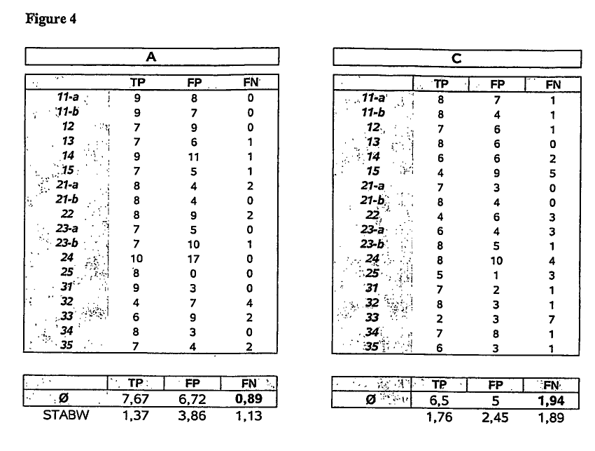

A more comprehensive view on the results is given in FIG. 3 and 4.

By applying the Wilcoxon rank sum test for the determination of false positives or false negatives as follows, it becomes evident that the optimized set resulted in a more reliable amplification experiment:
data: False negatives (FN)
p-value=0.02602 rejection of null hypothesis null hypothesis (H0): true if median of designed set equal or greater than of control set alternative hypothesis (H1): true if median of designed set less than of control set
data: False positives (FP)
p-value=0.06711 rejection of null hypothesis null hypothesis (H0): true if median of designed set equal or less than of control set alternative hypothesis (H1): true if median of designed set greater than of control set
data: True positives (TP)
p-value=0.02146 rejection null hypothesis null hypothesis (H0): true if median of designed set equal or less than of control set alternative hypothesis (H1): true if median of designed set greater than of control set

FIG. 3 illustrates a summary of several such comparisons (as described in detail above). Six diagrams are shown, that illustrate the numbers of false positives (FP), false negatives (FN) and true positives (TP) for a number of 18 experiments. In the top row of FIG. 3 the results for experiments that employed the design method are shown whereas in the lower row results from experiments are shown, that did use the conventional method of random selection.

At the x-axis the occurrence of an event (like a false positive) per 8plex is given whereas the values of the y-axis indicate the frequency of an event like this occurring within the number of experiments performed.

For example, in the diagram title FN, a y-value of 0 indicates that the event did not occur in a s ingle experiment, a y-value of four indicates that the according number of occurrences given as the x-value was found in four experiments (out of the 18 experiments considered for these analyses). The x-value indicates what kind of occurrence is counted; a x-value of three in this diagram indicates the occurrence of three false negatives. A data point with an x-value of 0 and an y-value of 9 means, that in the set of mPCR results considered, nine experiments showed 0 false negatives.

FIG. 4 gives all of the data from the 18 multiplex PCR experiments of this example in one table. The letter A, heading the four columns presented on the left side, is indicating the results from multiplex PCRs of the designed group using the five optimized sets of primer pairs that have been designed and selected according to the invention. The letter C is indicating the results from multiplex PCRs of the control group using the five randomized sets of primer pairs.

The first column lists the identifying numbers of the experiments, the second column gives the numbers of true positives (TP) within this experiment, the third column gives the numbers of false positives (FP) and the last column gives the numbers of false negatives (FN).

The average false negative rate (Ø FN) of the optimized group is significantly lower than in the control group. Complementary the average true positive rate (Ø TP) is significantly higher. The average false positive rates (Ø FP) of the two sets do not differ from each other significantly.

This is due to the high deviation of false positives observed between individual ALFexpress analysis runs. Those 36 sets of amplificates have been analyzed on two separate gel runs These runs were not designed to simply duplicate the results, but could be used to analyze whether the average TP, FP and FN rates are similar, independent of the run, and the sets chosen. Only three of those sets have been duplicated, as indicated by the letters a and b for sets 11, 21 and 23. It turned out that the rate of true positives as well as the rate of false negatives averaged over 18 sets per run were highly reproducible, 6.83 versus 7.33 and 1,44 versus 1.39 respectively. However, the rate of false positives was determined as 4.11 in the first run and 7.61 in the second run.

Taken together, it could be concluded that the overall success rate of amplifying 40 fragments within 5 groups of 8plex PCRs was significantly increased when the primer grouping was based on the method being subject of this invention compared to an arbitrary primer grouping. The improved success rate of only 11% failures versus 24% in the random control group clearly becomes relevant when much larger numbers of mPCRs have to be established as is the case in a high throughput laboratory.

1. A method for the amplification of nucleic acids comprising the following steps
1) isolating a nucleic acid sample,
2) treating said sample in a manner that differentiates between methylated and unmethylated cytosine bases within said sample,
3) amplifying at least one target sequence, within said treated nucleic acid, by means of enzymatic amplification and a set of primer molecules, wherein said primer molecules are characterized in that
a) each primer molecule sequence reaches a predefined measure of complexity,
b) every combination of any two primer molecules in the set has a melting temperature below a specified threshold temperature,
c) every combination of two primer molecules, under conditions allowing for one or more base mismatches per primer, does not lead to the amplification of an unwanted product when virtually tested using the treated and the untreated sample nucleic acids as template, and
4) detecting said amplified target nucleic acid.

2. A method according to claim 1 wherein said primer molecules do not contain nucleic acid sequences complementary or identical to nucleic acid sequences of the target sequence which prior to treatment of step 2 contained a 5′-CG-3′ site.

3. A method according to claim 1 wherein said set is comprised of at least one but not more than 32 primer pairs.

4. A method according to claim 1 wherein said set is comprised of at least one but not more than 16 primer pairs.

5. A method according to claim 1 wherein the primer molecules are reaching a specified value of linguistic complexity.

6. A method according to claim 1 wherein the primer molecules are reaching a specified value of Shannon entropy.

7. A method according to claim 1 wherein the nucleic acid sample is isolated from a bodily fluid, a cell culture, a tissue sample or a combination thereof.

8. A method according to claim 1 wherein the nucleic acid sample is comprised of plasmid DNA, BACs, YACs or genomic DNA.

9. A method according to claim 1 wherein the nucleic acid sample is comprised of human genomic DNA

10. A method according to claim 1 wherein said sample is treated by means of a solution of a bisulfite, hydrogen sulfite or disulfite.

11. A method according to claim 1 wherein said primer molecule comprises of at least one nucleotide within the last three nucleotides from the 3′ end of the molecule wherein said nucleotide is complementary to a nucleotide of the target sequence that was converted to a different nucleotide by the treatment performed in step 2) of claim 1.

12. A method according to claim 1 wherein said primer molecule comprises of at least one nucleotide within the last three nucleotides from the 3′ end of the molecule wherein said nucleotide is complementary to a nucleotide of the target sequence that was converted to a different nucleotide by bisulfite treatment.

13. A method according to claim 1 wherein each of said primer molecules is characterized in that the last at least 5 bases at the 3′ end of said primer molecule are not complementary to the sequence of any other primer molecule in the set.

14. A method according to claim 1 wherein the number of mismatches allowed for when virtually testing the amplification of unwanted products according to step 3 c) of claim 1 is less than 20% of the number of nucleotides of the primer molecule.

15. A method according to claim 1 wherein the number of nucleotides creating one gap, when aligning the primer molecule sequence with the template sequence, allowed for, when virtually testing the amplification of unwanted products according to step 3 c) of claim 1 is less than 20% of the number of nucleotides of the primer molecule.

16. A method according to claim 1 wherein the number of mismatches allowed for when virtually testing the amplification of unwanted products according to step 3 c) of claim 1 is less than 10% of the number of nucleotides of the primer molecule.

17. A method according to claim 1 wherein the number of nucleotides creating one gap, when aligning the primer molecule sequence with the template sequence, allowed for, when virtually testing the amplification of unwanted products according to step 3 c) of claim 1 is less than 10% of the number of nucleotides of the primer molecule.

18. A method according to claim 1 wherein the number of mismatches allowed for when virtually testing the amplification of unwanted products according to step 3 c) of claim 1 is less than 5% of the number of nucleotides of the primer molecule.

19. A method according to claim 1 wherein the number of nucleotides creating one gap, when aligning the primer molecule sequence with the template sequence, allowed for, when virtually testing the amplification of unwanted products according to step 3 c) of claim 1 is less than 5% of the number of nucleotides of the primer molecule.

20. A method according to claim 1 wherein the number of mismatches allowed for when virtually testing the amplification of unwanted products according to step 3 c) of claim 1 is less than seven.

21. A method according to claim 20 wherein the number of mismatches allowed for is less than five.

22. A method according to claim 20 wherein the number of mismatches allowed for is less than three.

23. A method according to claim 20 wherein the number of mismatches allowed for is one.

24. A method according to claim 1 wherein the number of mismatches allowed for when virtually testing the amplification of unwanted products according to step 3 c) of claim 1 is determined by a pre-specified maximum melting temperature.

25. A method according to claim 1 wherein said primer molecules are used to amplify nucleic acid sequences that prior to treatment of step 2 comprised of more than eight 5′-CG-3′ sites.

26. A method according to claim 1 wherein said primer molecules are used to amplify nucleic acid sequences that prior to treatment of step 2 comprised of more than six 5′-CG-3′ sites.

27. A method according to claim 1 wherein said primer molecules are used to amplify nucleic acid sequences that prior to treatment of step 2 comprised of more than four 5′-CG-3′ sites.

28. A method according to claim 1 wherein said primer molecules are used to amplify nucleic acid sequences that prior to treatment of step 2 comprised of more than two 5′-CG-3′ sites.

29. A method according to claim 1 wherein the ability of said primer molecules to amplify an unwanted product is tested by means of electronic PCR.

30. A method according to claim 1 wherein the ability of said primer molecules to amplify an unwanted product is tested by means of electronic PCR, taking as template nucleic acid the coding strand of the treated sample, the non-coding strand of the treated sample and both of the strands of the untreated sample.

31. A method according to claim 1 wherein the ability of said primer molecules to amplify an unwanted product is tested by means of electronic PCR, taking as template nucleic acid the coding strand of the bisulfite converted human genome, the non-coding strand of the bisulfite converted human genome and both of the strands of the untreated human genome.

32. A method according to claim 1 wherein said primer molecules are used to amplify nucleic acids which are comprised of at least 50 bp but not more than 2000 bp.

33. A method according to claim 1 wherein said primer molecules are used to amplify nucleic acids which are comprised of at least 80 bp but not more than 1000 bp.

34. A method according to claim 1 wherein said primer molecules are comprised of 16-50 nucleotides.

35. A method according to claim 1 wherein said primer molecules do not form dimers with each other.

36. A method according to claim 1 wherein said primer molecules do not form loops or hairpin structures.

37. A method according to claim 1 wherein said primer molecules are complementary to target sequences which prior to the treatment performed in step 2) of claim 1 did not contain specified restriction enzyme recognition sites.

38. A method according to claim 1 wherein said primer molecules amplify regions of the treated nucleic acids which prior to the treatment performed in step 2) of claim 1 did not contain specified restriction enzyme recognition sites.

39. A method for designing primers according to claim 1, comprising the steps of
a) selecting a pool of possible primer pairs per amplificate by means of a standard PCR primer design program using said nucleic acids as template
b) excluding those primer pairs which comprise of a primer that in combination with another primer molecule in the same set exceeds a threshold melting temperature
c) excluding those primer pairs which comprise of a primer that does not reach a specified level of complexity
d) excluding those primer pairs which comprise of a primer that in combination with another primer molecule in the same set, under conditions allowing for one or more base mismatches per primer, amplifies an unwanted product when virtually tested using the treated and the untreated sample nucleic acid as template.

40. A method for designing said primer molecules according to claim 1, adding the step of
e) excluding from the remaining confirmed primer pairs those pairs which in said amplification step do not result in the amplification of the intended product when performing a single PCR experiment.

41. A method for designing primers according to claim 39, wherein said template nucleic acids are masked for repeats and SNPs before designing said primer molecules and wherein said standard PCR primer design program considers one or more of the following factors
length of amplificate, length of primer, melting temperature of the primers, dimer formation parameters, loop formation parameters, exclusion of unidentified or ambiguous nucleotides in the primer sequence, exclusion of restriction enzyme recognition sites.

42. A method according to claim 39 wherein said measure of complexity is a measure of linguistic complexity.

43. A method according to claim 39 wherein said measure of complexity is a measure of Shannon entropy.

44. A method according to claim 39 wherein the following step is carried out prior to performing step d)
excluding from the remaining primer pairs those pairs, which consist of a primer molecule that comprises of at least one CpG site.

45. A method according to claim 39 wherein the following step is carried out prior to performing step d)
excluding from the remaining primer pairs those pairs, which consist of a primer molecule that does not contain at least one nucleotide within the last three nucleotides from the 3′ end of the molecule wherein said nucleotide is complementary to a nucleotide of the target sequence that was converted to a different nucleotide by the treatment performed in step 2).

46. A method according to claim 39 wherein the following step is carried out prior to performing step d)
excluding from the remaining primer pairs those pairs, which consist of a primer molecule that contains more than 5 bases at its 3′ end that are complementary to any other primer molecules' sequence in the set.

47. A method according to claim 39 wherein the following step is carried out prior to performing step d)
excluding from the remaining primer pairs those pairs, which amplify a nucleic acid that did not, prior to the treatment in step 2, contain at least two CpG sites.

48. A method according to claim 39 wherein the following step is added before performing step d)
excluding from the remaining primer pairs those pairs, which comprise of one primer molecule that in combination with another primer molecule in the set amplifies an unwanted product, when virtually testing according to step 3 c) of claim 1 under conditions allowing for a number of mismatching nucleotides of 20% of the number of nucleotides of the primer molecule.

49. A method according to claim 39 wherein the following step is added before performing step d)
excluding from the remaining primer pairs those pairs, which comprise of one primer molecule that in combination with another primer molecule in the set amplifies an unwanted product, when virtually testing according to step 3 c) under conditions allowing for a number of nucleotides creating one gap, when aligning the primer molecule sequence with the template sequence, of up to 20% of the number of nucleotides of the primer molecule.

50. A method according to claim 39 wherein the following step is added before performing step d)
excluding from the remaining primer pairs those pairs, which comprise of one primer molecule that in combination with another primer molecule in the set amplifies an unwanted product, when virtually testing according to step 3 c) under conditions allowing for four or less mismatching base pairs.

51. A method according to claim 39 wherein the following step is added before performing step d)
excluding from the remaining primer pairs those pairs, which comprise of one primer molecule that in combination with another primer molecule in the set amplifies an unwanted product, when virtually testing according to step 3 c) under conditions allowing for two or less mismatching base pairs.
